SEQUENCE LISTING (8 sequences)

SEQ ID NO: 1 - DNA, 456 bp, Homo sapiens
ggatccccaccaacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggctgtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgagccaccaccaccagaattc

SEQ ID NO: 2 - DNA, 78 bp, Homo sapiens
gcaggatccggaggagcagcagttgcactactaccagcagttctactagcactactagcaccaggaggagaattcgca

SEQ ID NO: 3 - DNA, 63 bp, Homo sapiens
gcaggatccggaggatatgcacgtgcagcagcacgtcaagcacgtgcaggaggagaattcgca

SEQ ID NO: 4 - DNA, 48 bp, Homo sapiens
cgccagattaaaatttggtttcagggacgccgcatgaaatggaaaaaa

SEQ ID NO: 5 - DNA, 33 bp, Human immunodeficiency virus
tacggtcgtaaaaaacgtcgtcagcgtcgtcgt

SEQ ID NO: 6 - DNA, 66 bp, Homo sapiens
atggtgaccgtgctgtttcgccgcctgcgcattcgccgcgcgtgcggcccgccgcgcgtgcgcgtg

SEQ ID NO: 7 - DNA, 63 bp, Homo sapiens
gcgggctatctgctgggcaaaattggactgaaagcgctggcggcgctggcgaaaaaaattctg

SEQ ID NO: 8 - DNA, 500 bp, Homo sapiens
ccaggttcaagggattctcccgcctcagcttccagagtagctgggactacagacacccaccaccatgcgtggctaatttttgtatttttagtagagagggggtttcgccatgttggccaggctggtttcaaactcctgacttcaggtgatccgcctgccacggcctcccaatttactgggattacaggggtgggccaccgcgcccggcctttttcttaatttttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttc

(19) United States
(12) Patent Application Publication
(10) Pub. No.: US 2017/0183633 A1
(43) Pub. Date: Jun. 29, 2017

(54) METHODS USING REPROGRAMMED CELLS FOR REGENERATIVE, RESTORATIVE, AND REJUVENATIVE THERAPIES

(71) Applicant: PrimeGen Biotech LLC, Santa Ana, CA (US)

(72) Inventors: Christian Kanemeier, Cardiff, CA (US); Joel Sae Won Marh, Portola Hills, CA (US); Kyle Howerton, Irvine, CA (US); John Sundsmo, Sausalito, CA (US)

(21) Appl. No.: 15/396,202

(22) Filed: Dec. 30, 2016

Related U.S. Application Data
(63) Continuation-in-part of application No. 12/671,683, filed on Nov. 4, 2011.
(60) Provisional application No. 60/953,395, filed on Aug. 1, 2007.
(60) Provisional application No. 60/974,325, filed on Sep. 21, 2007.
(60) Provisional application No. 61/024,836, filed on Jan. 30, 2008.
(60) Provisional application No. 61/030,514, filed on Feb. 21, 2008.
(60) Provisional application No. 61/060,363, filed on Jun. 10, 2008.

(51) Int. Cl. C12N 5/074; A61K 35/32; A61K 35/545; C12N 5/077; C12N 5/0793; A61K 35/28; A61K 35/34

(52) U.S. Cl. CPC C12N 5/0696; A61K 35/28; A61K 35/32; A61K 35/34; C12N 5/0653; C12N 5/0654; C12N 5/0655; C12N 5/0619; C12N 5/0657; A61K 35/545; C12N 2501/603; C12N 2501/606; C12N 2501/602; C12N 2501/604; C12N 2501/605; C12N 2501/065; C12N 2506/09; C12N 2506/02; C12N 2506/45; C12N 2506/08; C12N 2510/00

(57) ABSTRACT

Provided herein are methods of treatment to regenerate, restore or rejuvenate a tissue. Methods include making adult somatic and germ cells pluripotent for administration to a patient. Alternatively, created pluripotent cells may be differentiated to the desired tissue type and administered to a patient to repair or enhance the target tissue.

### CROSS REFERENCE TO RELATED APPLICATIONS

The present application is a continuation-in-part of U.S. patent application Ser. No. 12/671,683, filed Nov. 4, 2011 which is a U.S. National Stage of PCT/US08/72005, filed Aug. 1, 2008, which claims priority under 35 U.S.C. §119(e) to U.S. Provisional Patent Applications 60/953,395 filed Aug. 1, 2007, 60/974,325 filed Sep. 21, 2007; 61/024,836 filed Jan. 30, 2008, 61/030,514 filed Feb. 21, 2008 and 61/060,363 filed Jun. 10, 2008; the entire contents of all of which are incorporated herein by reference.

### FIELD OF THE INVENTION

The present invention relates to the field of reprogrammed cells. Specifically, reprogrammed cells can be used in an allogeneic or autologous manner and will function in the appropriate post-natal cellular environment to yield functional cells after transplantation. The invention relates generally to cellular compositions and methods useful in transplantation and specifically to stem cell-based therapeutics; and, most particularly to adult stem cell-based therapeutics. The invention provides compositions and methods for reprogramming adult somatic tissue cells to become at least multipotent stem cells that are similar to embryonic stem cells in their growth and differentiative capacities.

### BACKGROUND OF THE INVENTION

Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Scientists had long held the opinion that differentiated cells cannot be altered or caused to behave in any way other than the way in which have had been naturally committed. In recent stem cell experiments, however, scientists have been able to persuade blood stem cells to behave like neurons. Therefore, recent research has focused on ways to make multipotent cells into pluripotent types. Recent reports have suggested that this is possible when somatic cells are genetically modified by transduction with retroviruses encoding certain transcription factors. However, genetic modification is not presently considered a desirable therapeutic option and alternatives are needed.

Stem cells are a rare population of cells that can give rise to vast range of cells tissue types necessary for organ maintenance and function. These cells are defined as undifferentiated cells that have two fundamental characteristics; (i) they have the capacity of self-renewal, (ii) they also have the ability to differentiate into one or more specialized cell types with mature phenotypes. There are three main groups of stem cells; (i) adult or somatic (post-natal), which exist in all post-natal organisms, (ii) embryonic, which can be derived from a pre-embryonic or embryonic developmental stage and (iii) pre-natal stem cells (pre-natal), which can be isolated from the developing fetus. Each group of stem cells has their own advantages and disadvantages for cellular regeneration therapy, specifically in their differentiation potential and ability to engraft and function de novo in the appropriate or targeted cellular environment.

In the post-natal animal there are cells that are lineage-committed progenitor stem cells and lineage-uncommitted pluripotent stem cells, which reside in connective tissues providing the post-natal organism the cells required for continual organ or organ system maintenance and repair. These cells are termed somatic or adult stem cells and can be quiescent or non-quiescent. Typically adult stem cells share two characteristics: (i) they can make identical copies of themselves for long periods of time (long-term self renewal); and (ii) they can give rise to mature cell types that have characteristic morphologies and specialized functions.

Stem cells have reportedly been isolated from tissue types including brain, bone marrow, umbilical cord blood and amniotic fluid which appear to be multipotent at minimum. To date embryonic stem cells (ESC) have shown to be the most malleable stem cell source being pluripotent and having the ability to differentiate into any tissue type. However, there are noteworthy ethical concerns relating to possible creation of embryos solely for research purposes. In contrast, pre-natal stem cells may be donated from spontaneous or elective abortions; tissues would otherwise be discarded; and, they are not created for research purposes.

Much of the understanding of stem cell biology has been derived from hematopoietic stem cells and their behavior after bone marrow transplantation. There are several types of adult stem cells within the bone marrow niche, each having unique properties and variable differentiation ability in relation to their cellular environment. Somatic stem cells isolated from human bone marrow transferred in utero into pre-immune sheep fetuses have the ability to xenograft into multiple tissues. Also within the bone marrow niche are mesenchymal stem cells (MSC), which have a range of reported non-hematopoietic differentiation abilities, including bone, cartilage, adipose, tendon, lung, muscle, marrow stroma, and brain tissues. Despite their differentiative abilities, adult MSC generally appear to be multipotent, i.e., not pluripotent, and do not express markers characteristic of pluripotent ESC.

Another problem associated with using adult stems cells is that these cells are not immunologically privileged, or can lose their immunological privilege after transplant. (The term “immunologically privileged” is used to denote a state where the recipient's immune system does not recognize the cells as foreign). Thus, only autologous transplants are possible in most cases when adult stem cells are used. Thus, most presently envisioned forms of stem cell therapy are essentially customized medical procedures and therefore economic factors associated with such procedures limit their wide ranging potential.

Current research is focused on developing embryonic stem cells as a source of totipotent or pluripotent immunologically privileged cells for use in cellular regenerative therapy. However, since embryonic stem cells themselves may not be appropriate for direct transplant as they form teratomas after transplant, they are proposed as “universal donor” cells that can be differentiated into customized pluripotent, multipotent or committed cells that are appropriate for transplant. Additionally there are moral and ethical issues associated with the isolation of embryonic stem cells from human embryos.

Tissue cells had been believed to be “terminally differentiated”, i.e., cells irrevocably committed to their fate and function as lung, liver or heart cells. However, recently a few scientists have been reported that adult mouse and human somatic tissue cells can be encouraged in tissue culture with growth factors or through genetic manipulation to expand their potency and become capable of forming several different kinds of tissue cells. Unfortunately, a number of these approaches suffer from the disadvantage that the resultant cells may potentially form tumors. In addition, adult tissue cells are aged and subject to chromosomal oxidative and free radical damage and alterations such as telomere shortening. The latter genetic changes could substantially impact the future utility of such cells in patient therapies. Alternatives are highly desirable.

Much of the understanding of the possible uses of stem cells in therapy has derived from bone marrow transplantation of hematopoietic stem cells in patients with cancers and autoimmune diseases. Commonly, in these protocols the patient is treated with lethal levels of radiation and/or chemotherapy, i.e., to kill the cancer, and then the bone marrow and immune system, (destroyed by the cancer therapy), is reconstituted using either the patient's own bone marrow which has been rendered cancer-free in the laboratory (referred to as “autologous” for self derived bone marrow), or the bone marrow of a closely related donor (referred to as “allogeneic” for genetically closely related but not identical). Autologous (self) tissues are not subject to transplant rejection, but allogeneic tissues are subject to rejection. Fortunately, drugs are available for managing episodes transplant rejection and physicians have become very skilled in their uses. Unfortunately, in about half of the bone marrow transplant patients the grafted hematopoietic cells may populate the bone marrow, i.e., establishing a foreign immune system within the recipient. If the foreign immune system does not recognize the recipient as foreign, then it may establish what is referred to as a stable chimeric state. However, in about 30% to 65% of the recipients of bone marrow stem cell therapies (depending on the degree of HLA compatibility between the donor and recipient) the engrafted foreign immune system recognizes the recipient (host) as foreign and attempts to reject host tissues, referred to as graft versus host disease (GVHD). Again, physicians have become adept at managing GVHD, but are not always successful in all patients. Methods for transplanting patients with autologous tissue-derived stem cells would potentially alleviate many clinical problems in managing patients in transplant rejection and GVHD.

Stem cells in general have been reported to express low levels of transplantation antigens, i.e., genetically encoded by the major histocompatibility complex (MHC) and referred to as MHC class-I and class-II antigens. Low level antigen expression on stem cells may be advantageous in limiting immune recognition and transplant rejection. However, if stem cells administered to a patient have the capacity to differentiate into hematopoietic stem cells, then GVHD may still result. Clearly, therapeutic alternatives would be highly desirable.

The plasma membrane bilipid layer of cells protects, sustains and preserves cells by retaining important macromolecules, sensing the environment, transporting needed nutrients and inhibiting access of all but small molecules. Transfer of information across the cell membrane is essential for development, function and survival. Membrane receptors and transporters recognize and bind with specificity to growth-promoting factors, ions and nutrients. Transport occurs through endocytosis or receptor-mediated translocation. Translocation of pharmaceutically active compounds across the cell membrane is an aim in drug delivery. Unfortunately, large bioactive macromolecules like proteins are poorly translocated across the plasma membrane.

Single walled carbon nanotubes (SWNT) are carbon structures of sub-micron diameters and length that can be functionalized by an acid wash with carboxyl groups. This gives the SWNT the unique ability to bind to proteins non-specifically by hydrophobic interactions. Furthermore, SWNT can enter a cell, with proteins attached to it, by an endosomal route and thereby deliver their cargo into the cytoplasm of the cell, where the cargo can fulfill its function. This cargo can either consist of protein or DNA and thereby allows the efficient delivery of biologically active molecules into the cell. Alternatively to SWNT other sub-micron particles can be used, such as polyethylenimine particles, that fulfill the same function.

Cell penetrating peptides are hydrophobic amino acid sequences that, if attached to a protein molecule, can attach to a cell surface and facilitate entry into the cell by either an endosomal or non-endosomal route.

### SUMMARY OF THE INVENTION

Compositions and methods are provided for reprogramming adult and pre-natal somatic and germ-line cells to produce stem cell-like cells expressing embryonic stem cell (ESC) markers without the use of viruses. The methods involve introducing Oct-4 complex proteins, purified recombinant pluripotency factor proteins and mammalian expression plasmid DNAs encoding pluripotency factors into cells to up-regulate expression of embryonic stem cell genes. Methods are provided for determining that an adult somatic cell has been reprogrammed to produce at least a multipotent cell and ultimately an induced pluripotent stem (iPS) cell. Methods are provided for differentiating iPS cells into differentiated tissue cell types.

In one embodiment, a method is provided for producing reprogrammed cells comprising the steps of isolating a cell from a subject; introducing at least one pluripotency factor into the cell without the use of a virus to produce a reprogrammed cell; and determining that greater than 5% of the reprogrammed cells express at least one embryonic stem cell marker selected from the group consisting of Oct-4, Nanog, SSEA-3, SSEA-4, TRA1-60, Stellar, alkaline phosphatase and Rex-1.

In another embodiment, the at least one pluripotency factor is selected from the group consisting of transcription factor proteins, transcription factor DNAs, and transcription factor RNAs. In another embodiment, the at least one pluripotency factor is selected from the group consisting of Oct-4, c-Myc, Sox-2, Klf-4, Rybp, Zfp219, Sa114, Requiem, Arid 3b, P66β, Rex-1, Nac1, Nanog, Sp1, HDAC2, NF45, Cdk1 and EWS. In yet another embodiment, the at least one pluripotency factor comprises a mixture of Oct-4, c-Myc, Sox-2, Klf-4 and Nanog.

In another embodiment of the instant methods, the reprogrammed cell is pluripotent or multipotent. In another embodiment, the cell is selected from the group consisting of somatic cells, germ cells and post-natal stem cells. In another embodiment, the reprogrammed cell can differentiate into multiple cell lineages.

In yet another embodiment, the method further comprises the step of incubating the cell under conditions suitable for growth and progeny cell formation to form a continuous cell line.

In yet another embodiment, the method further comprises the addition of at least one of a demethylation agent and/or at least one of an acetylation agent in the introducing step. In another embodiment, the acetylation agent comprises valproic acid or a derivative thereof and the demethylation agent comprises 5-azacytidine.

In one embodiment, a therapeutic composition is provided comprising reprogrammed cells and a pharmaceutically acceptable carrier, wherein greater than 5% of the reprogrammed cells express an embryonic stem cell marker selected from the group consisting of Oct-4, Nanog, SSEA-3, SSEA-4, TRA1-60 and Rex-1 and wherein the reprogrammed cells were produced without the use of a virus.

In one embodiment, a composition is provided for reprogramming a cell to derive a multipotent or a pluripotent cell, comprising at least one pluripotency factor associated with a molecule that facilitates entry of the at least one pluripotency factor into the cell. In another embodiment, the at least one pluripotency factor is selected from the group consisting of transcription factor proteins, transcription factor DNAs, and transcription factor RNAs. In another embodiment, the at least one pluripotency factor is selected from the group consisting of Oct-4, c-Myc, Sox-2, Klf-4, Rybp, Zfp219, Sa114, Requiem, Arid 3b, P66β, Rex-1, Nac1, Nanog, Sp1, HDAC2, NF45, Cdk1 and EWS. In another embodiment, the composition comprises a single pluripotency factor DNA, RNA or protein bound to the molecule. In another embodiment, the composition comprises two or more pluripotency factor DNAs, RNAs or proteins bound to the molecule. In another embodiment, the at least one pluripotency factor is selected from the group consisting of Nanog and c-Myc, Oct-4 and c-Myc, Oct-4 and hTERT, Nanog and c-Myc, and Nanog and hTERT. In another embodiment, the at least one pluripotency factor comprises a mixture of Oct-4, c-Myc, Sox-2, Klf-4 and Nanog.

In yet another embodiment, molecule that facilitates entry of the at least one pluripotency factor into the cell is selected from the group costing of single walled nanotubes, cell penetrating peptides, polyethyleneimide particles and cationic amphiphile molecules. However, the molecule does not comprise a virus.

In one embodiment, an isolated reprogrammed cell is provided comprising a somatic cell reprogrammed by non-viral means to form a pluripotent or multipotent cell.

In another embodiment, a continuous culture of reprogrammed cells is provided comprising isolated somatic cells reprogrammed by non-viral means to form a continuous culture of pluripotent or multipotent cells.

### BRIEF DESCRIPTION OF THE DRAWINGS

FIGS. 1A-1E depict single walled nanotube (SWNT) based reprogramming of cells. FIG. 1A depicts human embryonic fibroblasts (HEF, HEF885 cells) transduced with IgG-GFP (green fluorescent protein) as disclosed further in Example 2. FIG. 1B depicts fluorescence activated cell sorting (FACS) analysis of the cells in FIG. 1A. FIG. 1C depicts HeLa cells before treatment. FIG. 1D depicts HeLa cells 48 hours after transduction with p53/SWNT. FIG. 1E depicts the growth of p53 knockout mouse embryonic fibroblasts (MEF) treated with p53/SWNT, untreated and SWNT only control.

FIGS. 2A-2F depict cells transduced with Oct-4 complex proteins or ESC lysate proteins bound to SWNT. FIG. 2A: HT42 NP-RFP cells before transduction; FIG. 2B: HT42 NP-RFP cells 11 days after transduction with Oct-4 complex proteins/SWNT; FIG. 2C: retinal pigment epithelial (RPE) cells 14 days post-transfection with Oct-4 complex proteins/SWNT;

FIG. 2D: human foreskin fibroblasts (HFF) 14 days post-transfection with Oct-4 complex proteins/SWNT; FIG. 2E: HT42 NP-RFP cells 14 days post-transfection with ESC lysate/SWNT; FIG. 2F: RPE cells 14 days post-transfection with ESC lysate/SWNT.

FIGS. 3A-3AH depict cells reprogrammed with pluripotent stem cell transcription factor DNAs bound to SWNTs (FIGS. 3A-3G) or polyethylenimide (PEI) particles (FIGS. 3H-3R). FIG. 3A: HFF cells treated with 5 transcription factor DNAs (Oct-4, Sox-2, Klf-4, Nanog and c-Myc; 5 TFactor DNAs) covalently attached to SWNT at Day 3; FIG. 3B: HEK cells treated with 5 TFactor DNA/SWNT at Day 6; FIG. 3C: RPE cells treated with 5 TFactor DNA/SWNT at Day 6; FIGS. 3D and 3E: SSEA-positive RPE cells at Day 14 after treatment with 5 TFactor DNA/SWNT; FIG. 3F: Colony formation from HT-42 cells treated with 5 TFactor DNA/SWNT at Day 6; FIG. 3G: Colony formation from HT-42 cells treated with 5 TFactor DNA/SWNT at Day 6 showing Nanog upregulation; FIG. 3H: HEF cells transfected with GFP plasmid DNA attached to PEI particles at Day 2; FIG. 3I: FACS analysis of HEF cells at Day 3; FIG. 3J: Expression pattern of transcription factors in HEF cells treated with 5 TFactor DNA/PEI particles before (0) and after (24, 28 and 72 hr) transfection; FIG. 3K: colony formation from HEF cells treated with 5 TFactor DNA/PEI particles on Day 23; FIG. 3L: cells from FIG. 3K further stained for SSEA-4; FIG. 3M: colony formation from HEF cells treated with 5 TFactor DNA/PEI particles on Day 48 and further stained for TRA1-60; FIG. 3N: colony formation from HEF cells treated with 5 TFactor DNA/PEI particles on Day 63 and further stained for alkaline phosphatase and human nuclei; FIGS. 30-3AE: multiplex RT-PCT gene expression analysis of the reprogrammed cells from FIG. 3N; FIG. 3AF: colony formation from HT42 cells treated with 5 TFactor DNA/PEI particles on Day 13; FIG. 3AG: the cells from FIG. 3P further showing RFP expression from the Nanog promoter locus; FIG. 3AH: colony formation from HT42 cells treated with 5 TFactor DNA/PEI particles on Day 56 further showing staining for alkaline phosphatase and human nuclei.

FIGS. 4A-4M depict cells reprogrammed with pluripotent stem cell transcription factor protein bound to SWNTs (FIGS. 4A-4H) or to a cationic amphiphile molecule (PULSin™ particles) (FIGS. 41-4M). FIG. 4A: colony formation from HFF cells on Day 6 after treatment with 5 TFactor proteins/SWNT; FIG. 4B: colony formation from HEK cells on Day 6 after treatment with 5 TFactor proteins/SWNT; FIG. 4C: colony formation from RPE cells on Day 13 after treatment with 5 TFactor proteins/SWNT; FIG. 4D: SSEA-4 positive colonies of RPE cells on Day 36 after treatment with 5 TFactor proteins/SWNT; FIG. 4E: colony formation from HT-42 cells on Day 6 after treatment with 5 TFactor proteins/SWNT; FIG. 4F: colony formation from HFF cells on Day 18 after treatment with 5 TFactor proteins/SWNT further showing Nanog up-regulation; FIG. 4G: SSEA-4 positive colonies of HT-42 cells on Day 38 after treatment with 5 TFactor proteins/SWNT; FIG. 4H: Alkaline phosphatase and human nuclei positive colonies of reprogrammed HT-42 cells on Day 53 after treatment with 5 TFactor proteins/SWNT; FIG. 4I: HEF cells transfected with Alexa 488 IgG/PULSin™ particles on Day 1; FIG. 4J: FACS analysis of the cells from FIG. 4I; FIG. 4K: colony formation from HEF cells transfected with 5 TFactor protein/PULSin™ particles at Day 29. FIG. 4L: SSEA-4 positive colonies from HEF cells transfected with 5 TFactor protein/PULSin™ particles at Day 55: FIG. 4M: colony formation from HT-42 cells transfected with 5 TFactor protein/PULSin™ particles at Day 6.

FIGS. 5A-5D depict purified cell penetrable peptides as vehicles for the non-viral reprogramming of cells. FIG. 5A-5C: HEF cells transduced with Oct-4 penetratin. FIG. 5D: FACS analysis of RFP cells transduced with pluripotency factors linked to cell penetrable peptides.

FIGS. 6A-6K depict ESC (FIGS. 6A and 6B) and HeLa cells (FIGS. 6C and 6D) transfected with the Nanog promoter linked to red fluorescent protein (RFP) and stained for RFP (FIGS. 6A and 6C) or Merge (FIGS. 6B and 6D). FIG. 6E-H: HeLa cells 48 hr after co-transfection with the Nanog promoter and 5 TFactor DNAs stained with RFP (FIGS. 6E and 6G) and Merge (FIGS. 6F and 6H); FIG. 6I: FACS analysis of the transfected HeLa cells at 48 hr; FIG. 6J: addition of the pluripotency factor Lin28 to the 5 TFactor DNA mixture in HeLa cells; FIG. 6K: Nanog activation after co-transfections of different combinatins of pluripotency factors in the presence and absence of valproic acid.

FIG. 7 depicts RPE and HEF cells treated with 5 TFactor proteins electrostatically attached to SWNT at various days after transfection and expressing pluripotent markers SSEA-3, SSEA-4, TRA1-81 and TRA1-60.

FIG. 8 depicts gene expression panel of retinal pigment epithelial cells grown in normal media before virus infection (Bar 1); RPE cells grown on mitomycin C treated mouse embryonic fibroblast feeder cells in hESC media at day 30 post infection with lentivirus containing Oct-4, Sox2, KLF4, c-Myc and Nanog virus (Bar 2) and RPE cells grown on mitomycin C-treated MEF feeder cells after two more rounds of subsequent virus infection with a combination of Oct-4, KLF4 and Sox2 lentivirus (Bar 3).

FIG. 9 depicts RPE colonies transduced with lentiviruses having a bicistronic construct containing either KLF4, Oct-4 or Sox2 in combination with GFP.

FIGS. 10A-10U depict a gene expression panel of human embryonic fibroblasts (HEF) HEF grown in normal culture media before virus infection (FIG. 10, Bar 1); grown in culture medium 6 days post infection (FIG. 10, Bar 2); grown in culture medium for 6 days post infection with lentiviral constructs containing KLF4, Sox2, Oct-4, Nanog and c-Myc and subsequently plated in hESC media on MEF feeder cells and grown for 11 days (FIG. 10, Bar 3). Established HEF iPS cell culture at day 30 post infection are depicted in FIG. 10, Bar 4.

FIGS. 11A-11C depict live cell imaging of newborn human foreskin fibroblasts (BJ) treated with pyrene butyrate (PB) (FIG. 11A); PBS or basal medium (FIG. 11B); or no treatment (FIG. 11C) followed by Oct4-TAT labeled with Alexa 488 (Oct4-TAT-488). The BJ cells were then imaged using microscopy channels at 10× magnification for bright field (BF) for optical illumination of the sample; 4′,6-diamidino-2-phenylindole (DAPI) staining to detect the PB; green fluorescent protein (GFP) to detect Oct4-TAT-488; and red fluorescence (TR) as a control to ensure the green and blue fluorescence are not auto-fluorescent.

FIGS. 12A-12C depict fixed cell imaging under 20× magnification of BJ cells treated with pyrene butyrate (PB) (FIG. 12A); PBS or basal medium (FIG. 12B); or no treatment (FIG. 12C) followed by Oct4-TAT labeled with Alexa 488 (Oct4-TAT-488).

FIG. 13 depicts eight examples of colony morphology of BJ cells after 5TF-TAT treatment.

FIGS. 14A-14D depict BJ colonies stained for SSEA-4 marker after 5TF-TAT treatment (FIGS. 14A-C) and control (FIG. 14D).

### DEFINITION OF TERMS

The following definition of terms is provided as a helpful reference for the reader. The terms used in this patent have specific meanings as they related to the present invention. Every effort has been made to use terms according to their ordinary and common meaning. However, where a discrepancy exists between the common ordinary meaning and the following definitions, these definitions supersede common usage.

Adult somatic cells: As used herein, “adult somatic cells” refer to cells isolated from individuals at any post-natal age.

Cell division cycle: As used herein, “cell division cycle” refers to the cell cycle process of preparing for and executing mitosis to duplicate a cell's genetic information and to form a daughter cell. Those skilled in the art recognize methods for determining the status of a cell within the cell cycle, e.g., for determining the stage in the cell cycle as being G₀, G₁, G₂ or M, as well as, determining that a cell has undergone DNA duplication and cell division to form a daughter cell.

Cell Penetrable Peptide: As used herein, “cell penetrable peptide”, abbreviated CPP, is intended to refer to a sequence of amino acids that, when covalently attached to a pluripotent stem cell transcription factor DNA, RNA, protein or protein complex, is effective to introduce the transcription factor(s) into the cytoplasm or endosomal compartment of a cell in a manner that delivers a cell reprogramming dose of pluripotent stem cell transcription factor protein(s) into the nucleus of the cell. In one embodiment, the instant CPP comprises a linear sequence of fewer than 45 amino acids, more preferably, the instant CPP comprises a linear sequence of fewer than 38 amino acids, and most preferably, the instant CPP comprises a linear sequence of fewer than 30 amino acids. Cell penetrable peptides are now well know in the art, e.g. as reviewed by U. Langel in “Cell Penetrating Peptides”, published in 2002 by Academic Press and incorporated herein by reference in its entirety. Molecular engineering techniques useful in constructing CPP are illustrated in the Examples section below.

Cell Reprogramming Dose: As used herein, “cell reprogramming dose” is intended to refer to the amount of pluripotent stem cell transcription factor DNA, RNA or protein or protein complex that, when delivered into a somatic cells, is effective to (a) induce colony formation; (b) unlimited growth and (c) cause the target cell to differentiate into any cell type in the mammalian body.

Cell surface marker: As used herein, “cell surface marker” means that the subject cell has on its cellular plasma membrane a protein, an enzyme or a carbohydrate capable of binding to an antibody and/or digesting an enzyme substrate. The cell surface markers are recognized in the art to serve as identifying characteristics of particular types of cells.

Committed: As used herein, “committed” refers to cells which are considered to be permanently epigenetically modified to fulfill a specific function in a tissue. Committed cells are also referred to as “terminally differentiated cells.”

Continuous cell culture: As used herein, “continuous cell culture” refers to cells in the subject tissue culture that can be passaged on a regular basis continuously in the laboratory, i.e., an immortalized cell line.

Dedifferentiation: As used herein, “dedifferentiation” refers to a process of cellular change resulting in an increase in a range of possible cellular functions from a narrow range of specialized functions to a broader range of possible cellular functions, e.g. from a single committed specific function to multiple different possible functions. Dedifferentiation leads to a less committed cell type.

Delivery Particle: As used herein, “delivery particle” is intended to refer to a particle capable of delivering one or more transcription factor DNAs, RNAs, proteins or protein complexes into a somatic cell in a manner effective to induce intrinsic reprogramming. Representative examples of delivery particles include, but are not limited to, carbon nanotubes such as single walled and multiwalled nanotubes; polysaccharide particles such as chitin, chitosan, polydextrin, cyclodextrin and agarose beads; magnetic particles; and the like. Preferably, the instant delivery particle has a size of less than about 5 nm in diameter and less than about 300 nm in length; more preferably, the instant delivery particle has a size of less than about 3 nm in diameter and less than about 350 nm in length; and, most preferably, the instant delivery particle has a size of less than about 1 nm in diameter and less than about 200 nm in length.

Differentiation: As used herein, “differentiation” refers to a process of systematic developmental changes, with accompanying epigenetic changes, that occur in cells as they acquire the capacity to perform particular specialized functions in tissues. In cells, differentiation leads to a more committed cell.

Embryo: As used herein, “embryo” refers to an animal in the early stages of growth and differentiation that are characterized implantation and gastrulation, where the three germ layers are defined and established and by differentiation of the germs layers into the respective organs and organ systems. The three germ layers are the endoderm, ectoderm and mesoderm.

Embryonic Stem Cell: As used herein, “embryonic stem cell” refers to any cell that is totipotent and derived from a developing embryo that has reached the developmental stage to have attached to the uterine wall. In this context, embryonic stem cell and pre-embryonic stem cell are equivalent terms. Embryonic stem cell-like (ESC-like) cells are totipotent cells not directly isolated from an embryo. ESC-like cells can be derived from precursor stem cells that have been dedifferentiated in accordance with the teachings disclosed herein.

Epigenetic: As used herein, “epigenetic” is intended to refer to the physical changes that are imposed in a cell upon chromosomes and genes wherein the changes affect the functions of the DNA and genes in the chromosomes and which do not alter the nucleotide sequence of the DNA in the genes. Representative examples of epigenetic changes include, but are not limited to, covalent chemical modifications of DNA such as methylation and acetylation as well as non-covalent and non-chemical modifications of DNA DNA super-coiling and association with chromosomal proteins like histones. Representative, non-limiting examples of the results of epigenetic changes include increasing or decreasing the levels of RNAs, and thereby protein products, produced by certain genes and/or changing the way that transcription factors bind at gene region sites termed “promoters”.

Epigenetic Imprinting: As used herein, “epigenetic imprinting” is intended to refer to the epigenetic changes imposed upon a DNA in the process of development and differentiation of a cell into a tissue. For instance, the changes imposed upon the DNA in a cell during development of, in non-limiting examples, a neural crest cell into a spinal cord or a brain cell, or development of a cardiomyocyte into cardiac muscle cell, or a keratinocye into a skin cell, or a myocyte into a skeletal muscle cell.

Expanding: When used in respect to the disclosed methods, “expanding” is intended to refer to the process for increasing the number of cells in a tissue culture. Representative methods for increasing the numbers of reprogrammed cells include tissue culture (a) in media containing one or more growth factors; (b) in conditioned media, e.g., “conditioned” by adding the subject media to cultures of embryonic stem cells; and/or (c) in the presence of “feeder” cells, e.g., mouse embryonic fibroblasts (MEFs) producing growth factors and extracellular matrix supportive of stem cell growth. The process of expanding cell numbers can be accomplished in tissue culture, in a bioreactor or in a cell-compatible implant. In the latter instance, the process involves reprogramming the somatic cells in vitro or in vivo and isolating and collecting the reprogrammed somatic cells into an implant material for return to the patient. In the latter process, the host incubates the reprogrammed cells inside the implant material, the implant material keeps the reprogrammed cells from differentiating back into somatic cells and the size of the subject implant material determines the size of the therapeutic unit dose administered to the patient.

Extrinsic Differentiation: As used herein, “extrinsic differentiation” refers to the process of introducing one or more reprogramming agents into the outside environment of a cell to effect a change in the cell from a less committed state to a more committed state. Representative examples of differentiation-inducing agents include, but are not limited to, tissue specific growth factors, their analogs, derivatives and chemical mimetics thereof. Representative examples of methods for inducing extrinsic differentiation with growth factors are illustrated in the Examples section.

Extrinsic reprogramming: As used herein, “extrinsic reprogramming” refers to the process of inducing an epigenetic genomic change in a somatic cell by introducing one or more extrinsic reprogramming agents into the outside environment of a somatic cell, wherein the epigenetic genomic change in the cell effects a change in the functional properties of the cell as evidenced by a change in the cell from a more committed state to a less committed state. Representative examples of extrinsic reprogramming agents include, but are not limited to, stem cell growth factors such as LIF, bFGF, EGF and the like, as well as, analogues, derivatives and chemical mimetics thereof. Representative examples of methods for effecting extrinsic reprogramming include introducing growth factor ligands into cell culture media, such as wherein the growth factor ligand binds to a cell surface receptor and triggers one or more signal transduction process that ultimately induce the epigenetic change in the cell.

Germ Line Stem Cells: As used herein, “germ line stem cells” refers to the conserved and protected multipotent, pluripotent and totipotent cells in the reproductive organs that insure the propagation of the species including, but not limited to, ovarian and testicular germ line stem cells.

Homogeneous: As used herein with regard to the instant iPS and RPSC compositions, “homogeneous” refers to cells that are uniformly distributed within the non-cellular components of the composition, e.g., uniformly distributed within a solution, an emulsion, a gel or a biodelivery matrix.

Induced Pluripotent Stem Cells: As used herein, “induced pluripotent stem cells” or iPS cells refers to an adult somatic cell that has been processed using intrinsic reprogramming methods to effect an epigenetic change from a “committed” and/or “terminally differentiated” state to a less committed state, such as, but not limited to a multipotent or “pluripotent” state.

Intrinsic Differentiation: As used herein, “intrinsic differentiation” refers to the process of introducing one or more differentiation-inducing agents into a cell to effect an epigenetic change in the cell from a less committed state to a more committed state. Representative examples of differentiation-inducing agents include, but are not limited to, tissue specific transcription factors like Myo-D, their analogs, derivatives and chemical mimetics thereof. Representative examples of methods for inducing intrinsic differentiation include, not not limited to, introducing a single walled nanotube (SWNT) into a cell that carries with it the Myo-D transcription factor thereby effecting a change in the commitment of the cell from a multipotent state to a muscle cell state.

Intrinsic Reprogramming: As used herein, “intrinsic reprogramming” refers to the process of introducing an intrinsic reprogramming agent into a somatic cell to induce an epigenetic genomic change in the cell that effects a change in the functional properties of the cell as evidenced by a change in the cell from a more committed state to a less committed state. Representative examples of intrinsic reprogramming agents include, but are not limited to, pluripotent stem cell transcription factors, as well as, analogues, derivatives and chemical mimetics thereof. Representative examples of methods for effecting intrinsic reprogramming appear in the Examples section and include introducing one or more pluripotent stem cell transcription factors into a cell. For the purposes of this disclosure, the term reprogramming includes both intrinsic reprogramming and therapeutic reprogramming.

Maturation: As used herein, “maturation” refers to a process of cellular change toward a more committed state. Representative non-limiting examples that such a process may be ongoing in an immature cell include evidence for biosynthesis of proteins such as enzymes and extracellular proteins present in the more committed cell type.

Multipotent: As used herein, “multipotent” refers to stem cells that can give rise to several other cell types, but those cell types are limited in number. An example of a multipotent stem cell is a hematopoietic stem cell such as a bone marrow stem cell that, while committed to develop into lineages of blood cells such as red and white blood cells, is lacking in the capacity to develop into other types of tissue cells, such as brain cells.

Multipotent Adult Progenitor Cells: As used herein, “multipotent adult progenitor cells” refers to multipotent cells isolated from the bone marrow which have the potential to differentiate into mesenchymal, endothelial and endodermal lineage cells. Oct-4 complex protein: As used herein “Oct-4 complex protein” refers to a protein that is associated with an Oct-4 protein in an embryonic stem cell extract. The instant Oct-4 complex proteins include, but are not limited to, Rybp, Zfp219, Sall4, Requiem, Arid 3b, P66β, Rex-1, Nac1, Nanog, Sp1, HDAC2, NF45, Cdk1 and EWS as well as proteins associated therewith. Representative examples of proteins that associate with Oct-4 complex proteins include, but are not limited to, Dax1, Mybbp, Etl1, Err2, Tif1β, Elys, Prmt1, Wdr18, REST, Rif1, BAF-155, Zfp281, Ral14, Sall1, Nac1, HDAC2, Wapl, Btbd14a, Zfp609, P66β, YY1, Rnf2, Pelo, Zfp198, Arid3b and Arid3a.

Passage: As used herein, “passage” refers to the process of splitting a growing cell culture into multiple different containers, e.g., one container into three containers (1:3 passage condition), so that the growth of the cells can continue in a new non-crowded space. Continuous cell cultures can be passaged in a routine manner indefinitely under the same passage conditions. Terminal cell cultures, e.g., of differentiated tissue cells, growth more slowly with time in tissue culture, i.e., requiring fewer and fewer passages and splitting to fewer and fewer containers.

Pluripotent: As used herein, “pluripotent” refers to cells that can give rise to any cell type except the cells of the placenta or other supporting cells of the uterus.

Pluripotent Stem Cell Culture: As used herein, “pluripotent stem cell culture” refers to a tissue culture preparation of cells obtained from an animal and serially passaged by splitting the growing cells into containers more than 20 times, preferably more than 30 times, more preferably greater than 60 times and most preferably greater than 100 times.

Pluripotent Stem Cell Transcription Factor: As used herein, “pluripotent stem cell transcription factor” or a “pluripotency factor” refers to a transcription factor expressed by a pluripotent stem cell and functionally involved in inducing or maintaining the epigenetic genomic state conducive to unlimited growth and differentiation of the pluripotent stem cell; and/or, directly involved in the unlimited growth potential of the pluripotent stem cell; and/or, involved in maintaining the capacity of the pluripotent stem cell to differentiate into a cell of an ectodermal, mesodermal or endodermal lineage. Representative examples of the instant pluripotent stem cell transcription factors include, but are not limited to, Oct-4, Sox-2, Klf-4, Nanog, c-Myc, Rybp, Zfp219, Sa114, Requiem, Arid 3b, P66β, Rex-1, Nac1, Nanog, Sp1, HDAC2, NF45, Cdk1, PLZF, cRET, Stellar, VASA and EWS. Embodiments disclosed herein provide methods for reprogramming cells in primary somatic cell cultures with pluripotent stem cell transcription factor DNAs, RNAs and proteins. In one embodiment, the pluripotency factors are referred to “5 transcription factor” or “5 TFactor” proteins or DNA. The 5 transcription factors are Oct-4, c-Myc, Sox-2, Klf4 and Nanog.

Post-natal Stem Cell: As used herein, “post-natal stem cell” refers to any cell that is multipotent and derived from a multi-cellular organism after birth.

Pre-natal Stem Cell: As used herein, “pre-natal stem cell” refers to a cell that is multipotent and derived from a developing multi-cellular fetus that is no longer in early or mid-stage organogenesis.

Primary Culture: As used herein, “primary culture” refers to a tissue culture preparation of cells obtained from an animal and serially passaged by splitting the growing cells into containers fewer than 100 times, preferably fewer than 60 times, more preferably fewer than 30 times, and most preferably fewer than 20 times.

Promoter: As used herein, “promoter” is used to refer to elements that are generally located in the 5′ region of genes, which bind transcription regulatory factors, and which binding alters the function of the gene by increasing or decreasing the amount of an RNA produced by the gene.

Regenerate: When used in regard to the instant therapeutic methods, “regenerate” is intended to refer to the process of rebuilding the structural cellular and extracellular elements of a diseased and/or aged tissue so that it is returned to a structure that is less-diseased and more normal and/or youthful.

Rejuvenate: When used in regard to the instant therapeutic methods, “rejuvenate” is intended to refer to the process of rendering an aged tissue more youthful and vibrant.

Reporter Cell Line: As used herein, “reporter cell line” is intended to refer to a plurality of reprogrammed somatic cells capable of unlimited self-renewal, constructed by instrinsic reprogramming of a normal or a diseased somatic cell, and containing one or more marker genetic elements. Representative examples of reporter cell lines are disclosed in the Examples section such as human testicular cells containing an RFP (red fluorescent protein) marker gene under the control of an Oct-4 promoter.

Reprogrammed Cell (RC): As used herein, “RC” refers to an adult somatic cell that has been processed using intrinsic or therapeutic reprogramming methods, to effect an epigenetic change from a “committed” and/or “terminally differentiated” state to a less committed state, a multipotent or pluripotent state. For the purposes of the instant disclosure, reprogrammed cells include those generated by both intrinsic and therapeutic reprogramming. That an adult somatic cell has been reprogrammed in an intrinsic reprogramming process to an RC is determined by assessing the expression of ESC stem cell markers, i.e., cell surface markers, mRNA markers or RT-PCR markers; or, assessing the potential for stable continuous growth in tissue culture passage; or, assessing the pluripotent differentiative functional capacity of the cells, i.e., to form cell types derived from the ectoderm (e.g., skin), mesoderm (e.g., organs) and endoderm (e.g., linings of the body cavities and blood vessels). Representative examples of ESC stem cell mRNA and RT-PCR and immunohistochemical markers include, but are not limited to, Oct-4, Nanog, SSEA-3, SSEA-4, TRA-1-60 Stellar, alkaline phosphatase and Rex-1. Representative examples of ESC stem cell surface markers include, but are not limited to, CD44, SSEA-4, CD105, CD166, CD90 and CD49f.

Reprogramming: As used herein “reprogramming” refers to the epigenetic genomic changes that result in a committed cell being induced to enter a less committed state. Representative examples include epigenetic changes sufficient to induce terminally differentiated somatic cell to exhibit functional properties of a multipotent or a pluripotent cell. For the purposes of the instant disclosure, reprogramming includes both intrinsic and therapeutic reprogramming.

Restore: When used in regard to the instant therapeutic methods, “restore” is intended to refer to the process of bringing the function of a tissue from a diseased or aged state back to a more normal and/or youthful state.

RT-PCR marker: As used herein with regard to a cell in a cell culture of RC, “RT-PCR marker” means that the subject cell has in its cellular cytoplasm an RNA that can be copied and amplified using a reverse transcriptase polymerase chain reaction (PCR) methodology. The subject RNAs are recognized in the art to serve as identifying characteristics of particular types of cells.

Somatic Cell: As used herein, “somatic cell” refers to any cell in a tissue in the mammalian body except gametes and their precursors. Representative examples include fibroblasts, epithelial cells, retinal pigment epithelial cells, lung epithelial cells, kidney proximal tubule cells.

Somatic Stem Cells: As used herein, “somatic stem cells” refers to diploid multipotent or pluripotent stem cells resident in a tissue in the mammalian body. Somatic stem cells are not totipotent stem cells and many are now understood not to be pluripotent. Representative examples include neural stem cells, kidney stem cells, muscle satellite stem cells, cartilage satellite stem cells and the like.

Substantially Purified: As used herein with regard to a cell composition, “substantially purified” means that, with regard to the cells in the composition, fewer than 25% are of a type other than the desired cell type; preferably, fewer than 15% are of a type other than the desired cell type; more preferably, fewer than 10% are of a type other than the desired cell type; and, most preferably, fewer than 5% are of a type other than the desired cell type.

Therapeutic Unit Dose: When used in reference to reprogrammed cells, “therapeutic unit dose” is intended to refer to that number of cells that is effective to regenerate, restore or rejuvenate a tissue to its natural non-diseased and/or non-aged state,

Totipotent: As used herein, “totipotent” refers to cells that have an epigenetic genomic state that allows them to differentiate into any cell type in any tissue of a mammalian body including the placenta. Without reprogramming, native human embryonic cells only have totipotent properties during the first few divisions after fertilization of an ovum (egg).

Transaction: As used herein, “transaction” is intended to refer to the process of delivering a pluripotent stem cell transcription factor DNA, RNA protein or protein transcription factor complex into a cell in a manner effective to induce intrinsic reprogramming of a somatic cell. Representative examples of transaction processes are disclosed in the Examples section below including, but not limited to, uses of particles for delivery, e.g. single and multi-walled nanotubes (SWNT), chitosan particles, cyclodextrin particles and the like. The instant transaction process delivers a cell reprogramming dose of one or more pluripotent stem cell transcription factor DNAs, RNAs, proteins and/or protein complexes into the nucleus of the cell in a manner effective to induce up-regulated expression of one or more genes having a promoter region that binds Oct-4, Sox-2, Klf-4, Nanog, c-myc, Rybp, Zfp219, Sall4, Requiem, Arid 3b, P66β, Rex-1, Nac1, Sp1, HDAC2, NF45, Cdk1 or EWS as well as proteins associated therewith in pluripotent stem cell transcription factor complexes.

Transcription Factor Complex: As used herein, “transcription factor complex” is intended to refer to the natural unassisted association of multiple different transcription factor proteins into an aggregate by virtue of the innate propensities of the different transcription factor proteins for one another. The Oct-4 transcription factor complex is one example of the self-association of the Oct-4 protein with other proteins including, but not limited to, Rybp, Zfp219, Sall4, Requiem, Arid 3b, P66β, Rex-1, Nac1, Nanog, Sp1, HDAC2, NF45, Cdk1, PLZF, cRET, Stellar, VASA and EWS.

### DETAILED DESCRIPTION OF THE INVENTION

The present disclosure provides biologically useful pluripotent therapeutically reprogrammed adult somatic cells and methods for preparation. The instant cells have pluripotent growth and differentiative capacities similar to embryonic stem cells (that is, ESC-like). Moreover, according to the methods of the invention therapeutically reprogrammed cells can be prepared for use in autologous therapies, i.e., where the cells are collected, reprogrammed and returned to the subject. Thus, in certain embodiments, the instant therapeutically reprogrammed cells are immunologically identical to the host and therefore suitable for therapeutic applications.

Stem cells are primitive cells that give rise to other types of cells. Also called progenitor cells, there are several kinds of stem cells. Totipotent cells are considered the “master” cells of the body because they contain all the genetic information needed to create all the cells of the body plus the placenta, which nourishes the human embryo. Human cells have this totipotent capacity only during the first few divisions of a fertilized egg. After three to four divisions of totipotent cells, there follows a series of stages in which the cells become increasingly specialized. The next stage of division results in pluripotent cells, which are highly versatile and can give rise to any cell type except the cells of the placenta or other supporting tissues of the uterus. At the next stage, cells become multipotent, meaning they can give rise to several other cell types, but those types are limited in number. An example of multipotent cells is hematopoietic cells—blood cells that can develop into several types of blood cells, but cannot develop into brain cells. At the end of the long chain of cell divisions that make up the embryo are “terminally differentiated” cells—cells that are considered to be permanently committed to a specific function.

Scientists had long held the opinion that differentiated cells cannot be altered or caused to behave in any way other than the way in which have had been naturally committed. In recent stem cell experiments, however, scientists have been able to persuade blood stem cells to behave like neurons. Therefore research has also focused on ways to make multipotent cells into pluripotent types.

Embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage.

For the purpose of this discussion, an embryo and a fetus are distinguished based on the developmental stage in relation to organogenesis. The pre-embryonic stage refers to a period in which the pre-embryo is undergoing the initial stages of cleavage. Early embryogenesis is marked by implantation and gastrulation, wherein the three germ layers are defined and established. Late embryogenesis is defined by the differentiation of the germ layer derivatives into formation of respective organs and organ systems. The transition of embryo to fetus is defined by the development of most major organs and organ systems, followed by rapid pre-natal growth.

Embryogenesis is the developmental process wherein an oocyte fertilized by a sperm begins to divide and undergoes the first round of embryogenesis where cleavage and blastulation occur. During the second round, implantation, gastrulation and early organogenesis takes place. The third round is characterized by organogenesis and the last round of embryogenesis, wherein the embryo is no longer termed an embryo, but a fetus, is when pre-natal growth and development occurs.

During embryogenesis the first two tissue lineages arising from the morulae post-cleavage and compaction are the trophectoderm and the primitive endoderm, which make major contributions to the placenta and the extraembryonic yolk sac. Shortly after compaction and prior to implanting the epiblast or primitive ectoderm begins to develop.

The epiblast provides the cells that give rise to the embryo proper. Blastulation is complete upon the development of the epiblast stem cell niche wherein pluripotent cells are housed and directed to perform various developmental tasks during development, at which time the embryo emerges from the zona pellucida and implants to the uterine wall. Implantation is followed by gastrulation and early organogenesis. By the end of the first round of organogenesis, all three germ layers will have been formed; ectoderm, mesoderm and definitive endoderm and basic body plan and organ primordia are established. Following early organogenesis, embryogenesis is marked by extensive organ development at which time completion marks the transformation of the developing embryo into a developing fetus which is characterized by pre-natal growth and a final round of organ development. Once embryogenesis is complete, the gestation period is ended by birth, at which time the organism has all the required organs, tissues and cellular niches to function normally and survive postnatally.

The process of embryogenesis is used to describe the global process of embryo development as it occurs, but on a cellular level embryogenesis can be described and/or demonstrated by cell maturation.

Pre-natal stem cells have been isolated from the pre-natal bone marrow (hematopoietic stem cells), pre-natal brain (neural stem cells) and amniotic fluid (pluripotent amniotic stem cells). In addition, stem cells have been described in both adult male and pre-natal tissues. Pre-natal stem cells serve multiple roles during the process of organogenesis and pre-natal development, and ultimately become part of the somatic stem cell reserve.

Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory.

During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandem repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress.

There is a history of cellular therapy for the treatment of a variety of diseases but the majority of the use has been in bone marrow transplantation for hematopoietic disorders, including malignancies. In bone marrow transplantation, an individual's immune system is restored with the transplanted bone marrow from another individual. This restoration has long been attributed to the action of hematopoietic stem cells in the bone marrow.

There is increasing evidence that stem cells can be differentiated into particular cell types in vitro and shown to have the potential to be multipotent by engrafting into various tissues and transit across germ layers and as such have been the subject of much research for cellular therapy. As with conventional types of transplants, immune rejection is the limiting factor for cellular therapy. The recipient individual's phenotype and the phenotype of the donor will determine if a cell or organ transplant will be tolerated or rejected by the immune system.

Therefore, the present disclosure provides methods and compositions for providing functional immunocompatible stem cells for cellular regenerative/reparative therapy.

The expression of pluripotent markers are indicative of cells that have the capacity to differentiate into all three germ layers. The transcription factors Oct-4, Nanog, and Sox-2 are expressed at high levels in ESC. Scientists presently believe that their expression indicates an undifferentiated pluripotent status in ESC.

Embodiments disclosed herein provide cellular compositions of reprogrammed cells (RC) in which greater than 5% of cells present express an ESC stem cell marker selected from the group consisting of Oct-4, Nanog, SSEA-3, SSEA-4, TRA-1-60 and Rex-1; preferably, greater than 10% of the cells express these ESC stem cell markers; more preferably, greater than 50% of the cells express these ESC stem cell markers; and, most preferably, greater than 75% of the cells express these ESC stem cell markers. In alternative embodiments, the instant cellular compositions are stable continuous cell cultures of RC; suspensions of cells; and, biodelivery devices containing cells e.g. prepared for therapeutic use in subjects in need thereof.

In alternative embodiments, the instant RC are derived by therapeutically reprogramming of adult somatic cells derived from humans, domesticated animals, wild mammals, birds and boney fishes.

The choice of adult somatic cells for derivation of the instant RC is of course at the discretion of the physician and patient and will vary depending upon at least the medical condition, age, location where the treatment is to be administered and chromosomal status, e.g., the extent of age-related DNA damage. Representative examples of adult somatic cells useful in the instant methods include ectodermal cells such as fibroblasts and epithelial cells; mesodermal organ cells such as bone marrow cells, CD34⁺ peripheral blood stem cells, cardiomyocytes, myocytes, vascular smooth muscle cells, hepatocytes and renal cells; and, endodermal endothelial cells such as vascular endothelial cells. In certain embodiments where age-related DNA damage is considered important, germ line stem cells are a possible preferred cell for production of RC and illustrative methods are provided in the Examples section.

Embodiments of the invention provide methods for producing RC involving the steps of obtaining a somatic cell sample from an adult or pre-natal subject; therapeutically reprogramming the adult somatic cells in the cell sample using an intrinsic reprogramming method that introduces an Oct-4 complex protein into a cell; and, verifying that the adult somatic cells are RC by testing for the expression of an ESC stem cell marker.

In other embodiments, methods are provided for transaction of cells by delivery of pluripotent stem cell transcription factor DNAs, RNAs, proteins and protein transcription factor complexes into endosomes and phagosomes, or alternatively, through the plasma membrane and into the cytoplasm of cells in a manner effective to induce intrinsic reprogramming of somatic cells. The instant delivery methods include uses of delivery particles to which the subject DNAs, RNAs, proteins are protein complexes are coupled, as well as, in alternative embodiments, the use cell penetrable peptides to which transcription factor DNAs and/or RNAs are attached and wherein transcription factor recombinant proteins and protein complexes are constructed either to contain and/or attach cell penetrable peptides.

In other embodiments, reporter cell lines and processes for constructing such cell lines are provided. Reporter cell lines find a variety of uses in medicine including screening for pharmaceutical compounds that alter gene expression. Representative examples of reporter cell lines are disclosed in the examples section below, e.g., human testicular cells containing an RFP (red fluorescent protein) marker gene under the control of an Oct-4 promoter. Other examples of reporter cell lines include intrinsically reprogrammed somatic cells containing markers for up-regulation of apoptotic genes including e.g., calpain and cdk5/p25; alteration of oxygen homeostasis, e.g. HIF-1; changed mitochondrial function, e.g., PGC-1; cytoprotection, e.g., ALDH1A1, ALDH1A7, BIRC5/surviving, GST M5, GST A2, GST P1, NAD(P) quinine reductase (NQO1) and Nrf2; adipocyte/fat development, e.g., SRC-3; induction of immune tolerance, e.g., FoxP3; and, induction of immune T-helper cells, e.g., STAT6 or GATA-3.

In other embodiments, methods are provided for treating a subject in need of regenerative, restorative or rejuvenative stem cell therapy with autologous stem cells that obviate problems of transplant rejection and graft versus host disease. The method involves collecting a tissue sample from the subject; isolating somatic cells from the tissue; reprogramming the isolated somatic cells to produce multipotent or pluripotent stem cells; expanding the numbers of the reprogrammed cells to produce a therapeutic unit dose; and, (a) if the aim of the therapy is to provide a stem cell therapy, then returning the cells to the subject, or alternatively, (b) if the aim of the therapy is to provide a differentiated cell therapy, then differentiating the reprogrammed stem cells back into a somatic cell before returning the cells to the subject. The instant therapeutic method solves a significant problem inherent in tissue transplantation therapies: namely, in most cases because somatic cells are terminally differentiated, they cannot be successfully propagated in tissue culture under conditions that will enable production of a therapeutic unit dose. As a result, it is at present common to transplant patients with cells derived from another individual, e.g., cadaveric cells or cord blood cells. Reprogramming somatic cells restores their potential for unlimited growth without producing cancerous cells. While not wishing to be tied to any particular mechanism(s), it is presently believed that the intrinsic reprogramming methods presented herein preserve the epigenetic imprinting of the original tissue of origin. For example, skin cells that are intrinsically reprogrammed “remember” via their epigenetic imprinting that they are skin cells and not cancer cells. As a result, when they are expanded and transplanted back into their host they have the imprinting to differentiate back into skin cells and not into cancer cells. This solves an important safety issue in cell-based therapies, i.e., the major safety issue restricting the widespread use of embryonic stem cells in human treatments.

Representative examples of therapies using the instant methods for autologous regenerative, restorative and rejuvenative cell therapies include the following:

1. Treatments for age-related macular degeneration (both the wet and dry forms) involving collecting retinal pigment epithelial (RPE) cells from the eye of a patient with the disease, intrinsically reprogramming the RPE cells, expanding the cells to produce a therapeutic unit dose, and (a) if stem cell therapy is the objective, delivering the therapeutic unit dose of reprogrammed cells to the patient, or alternatively, (b) if differentiated cell therapy is the objective, then re-differentiating the reprogrammed cells back into RPE before delivery to the patient.

2. Treatments for Type-1 insulin-dependent diabetes mellitus (IDDM), or Type-2 diabetes, involving collecting islet cells (α, β, γ and the like) from the pancreas of a new-onset patient, intrinsically reprogramming the islet cells, expanding the reprogrammed cells to produce a therapeutic unit dose and (a) if the objective in the therapy is to provide a stem cell therapy, then delivering the therapeutic unit dose of the reprogrammed cells to a tissue location in the patient, or alternatively, (b) if the objective in the therapy is to provide a differentiated cell therapy, then differentiating the reprogrammed islet cells back into specialized islet cells, e.g. α, β, γ and the like, before delivery of the therapeutic unit dose to the tissue location in the patient. The subject tissue location to which the therapy is delivered in the patient may be the same or different from the origin of the tissue sample. For instance, the somatic cells may be collected from the pancreas and returned to other sites including, but not limited, to sites in the liver, skin or kidney capsule.

3. Treatments for bone marrow reconstitution using autologous peripheral blood stem cells involving collecting and purifying peripheral blood stem cells (such as, but not limited to, CD34+ cells) from a patient prior to radiation and/or chemotherapy, intrinsically reprogramming the subject cells, expanding the reprogrammed stem cells to produce a therapeutic unit dose, and delivering the therapeutic unit dose of the reprogrammed cells to the patient after the radiation and/or chemotherapy. By way of explanation, CD34+ stem cells in peripheral blood offered great hope in the 1990's for autologous reconstitution of the bone marrow in patients with hematological malignancies after whole body radiation and/or chemotherapy. Unfortunately, as the collected cells were expanded in tissue culture they tended to differentiate. When the subject cells were returned to patients the bone marrow was reconstituted for only a few months. Thus, what appeared initially to hold great promise for individualized bone marrow reconstitution failed to meet its clinical objectives. The instant methods solve these problems.

4. Treatments for non-union bone fractures, involving collecting osteocytes and osteoblasts from a patient, intrinsically reprogramming the cells; expanding the cells to produce a therapeutic unit dose, and (a) if the objective is stem cell therapy, delivering the therapeutic unit dose of the reprogrammed cells to the patient, or alternatively, (b) if the objective is differentiated cell therapy, differentiating the reprogrammed cells back into osteocytes and osteoblasts before delivery of the therapeutic unit dose to the patient.

Importantly, in a clinical setting it is often difficult to obtain large numbers of cells from a patient, such as 3000-6000 retinal pigment epithelial cells from a patient with age related macular degeneration or a few thousand islet cells derived from 10-15 isolated pancreatic islets. Reprogramming must therefore be highly efficient to enable expansion of relatively small numbers of cells into the numbers of cells required to enable a therapeutic unit dose. With viral transduction the efficiency of reprogramming is commonly less than about 1%. Since four or five transcription factors need to be expressed to effect reprogramming, the theoretical efficiency for five factor viral reprogramming would be a five factorial of 0.1-1% or less than about 0.00001%. In contrast, the instant intrinsic reprogramming methods yield efficiencies for five transcription factor reprogramming at greater than about 1% efficiency, preferably greater than about 5% efficiency and most preferably greater than about 10% efficiency. This high efficiency enables, for the first time, autologous stem cell therapies using reprogrammed adult somatic cells.

The route of delivery according to the instant methods is determined by the disease and the site where treatment is required. For topical application, it may prove desirable to apply the instant cellular compositions at the local site (such as by placing a needle into the tissue at that site or by placing a timed-release implant or patch); while in a more acute disease clinical setting it may prove desirable to administer the instant cellular compositions systemically. For other indications the instant cellular compositions may be delivered by intravenous, intraperitoneal, intramuscular, subcutaneous and intradermal injection, as well as, by intranasal and intrabronchial instillation (including, but not limited to, with a nebulizer), transdermal delivery (e.g., with a lipid-soluble carrier in a skin patch), or gastrointestinal delivery (e.g., with a capsule or tablet). The preferred therapeutic cellar compositions for inocula and dosage will vary with the clinical indication. The inocula may typically be prepared from a frozen cell preparation such as by thawing the cells and suspending them in a physiologically acceptable diluent such as saline, phosphate-buffered saline or tissue culture medium. Some variation in dosage will necessarily occur depending upon the condition of the patient being treated, and the physician will, in any event, determine the appropriate dose for the individual patient. Since the pharmacokinetics and pharmacodynamics of the instant cellular compositions will vary somewhat in different patients, the most preferred method for achieving a therapeutic concentration in a tissue is to gradually escalate the dosage and monitor the clinical effects. The initial dose, for such an escalating dosage regimen of therapy, will depend upon the route of administration.

The instant cellular compositions may to be administered alone or in combination with one or more pharmaceutically acceptable carriers, in either single or multiple doses. Suitable pharmaceutical carriers may include inert biodelivery gels or biodegradable semi-solid matrices, as well as diluents or fillers, sterile aqueous solutions and various nontoxic solvents. The subject pharmaceutically acceptable carriers generally perform three functions: namely, (1) to maintain and preserve the cells in the instant cellular composition; (2) to retain the cells at a tissue site in need of regeneration, restoration or rejuvenation; and, (3) to improve the ease of handling of the instant composition by a practitioner, such as, but not limited to, improving the properties of an injectable composition or the handling of a surgical implant. The pharmaceutical compositions formed by combining an instant cellular composition with a pharmaceutically acceptable carrier may be administered according to the instant methods in a variety of dosage forms such as syrups, injectable solutions, and the like. The subject pharmaceutical carriers can, if desired, contain additional ingredients such as flavorings, binders, excipients, and the like. For certain gastrointestinal procedures it may be desirable to encapsulate the instant cellular composition to protect the cells during passage through the stomach, e.g., in hard-filled gelatin capsules. For this purpose capsules might additionally include additives such as lactose or milk sugar and/or polyethylene glycols as cellular preservatives. For parenteral administration according to the instant methods, solutions may be prepared in sesame or peanut oil or in aqueous polypropylene glycol, as well as sterile aqueous isotonic saline solutions. The subject aqueous solution is preferably suitably buffered if necessary and the liquid diluent first rendered isotonic with sufficient saline or glucose. Such aqueous solutions of instant cellular composition may be particularly suitable for intravenous, intramuscular, subcutaneous, and intraperitoneal injection. The subject sterile aqueous media employed are obtainable by standard techniques well known to those skilled in the art. For use in one or more of the instant methods, it may prove desirable to stabilize a instant cellular composition, such as, but not limited to, increasing shelf life, viability and efficacy. Methods for preserving, storing and shipping frozen cells in preservative solutions are known in the art. Improving the shelf-life stability of cell compositions, e.g., at room temperature or 4° C., may be accomplished by adding excipients such as: a) hydrophobic agents (e.g., glycerol); b) non-linked sugars (e.g., sucrose, mannose, sorbitol, rhamnose, xylose); c) non-linked complex carbohydrates (e.g., lactose); and/or d) bacteriostatic agents or antibiotics.

The preferred pharmaceutical compositions for inocula and dosage for use in the instant methods will vary with the clinical indication. The inocula may typically be prepared from a concentrated cell solution by the practicing physician at the time of treatment, such as by thawing and then diluting a concentrated frozen cell suspension in a storage solution into a physiologically acceptable diluent such as phosphate-buffered saline or tissue culture medium. Some variation in dosage will necessarily occur depending upon the condition of the patient being treated, and the physician will, in any event, determine the appropriate dose for the individual patient.

The effective amount of the instant cellular composition per unit dose depends, among other things, on the body weight, physiology, and chosen inoculation regimen. A unit dose of the instant cellular composition refers to the number of cells in the subject suspension. Generally, the number of cells administered to a subject in need thereof according to the practice of the invention will be in the range of about 10⁵/site to about 10⁹/site. Single unit dosage forms and multi-use dosage forms are considered within the scope of the present disclosure.

For treatments of local dermal reconstructive and cosmetic clinical indications, the instant cellular composition may be provided in an emollient cream or gel. Representative examples of non-toxic cell-preservative emollient pharmaceutically acceptable carriers include cell-oil-in-water and cell-water-in-oil emulsions, i.e., as are known to those skilled in the pharmaceutical arts.

In alternative embodiments, the present disclosure provides different routes for delivery of the instant cellular compositions as may be suitable for use in the different disease states and sites where treatment is required. For topical, intrathecal, intramuscular or intra-rectal application it may prove desirable to apply the subject cells in a cell-preservative salve, ointment or emollient pharmaceutical composition at the local site, or to place an impregnated bandage or a dermal timed-release lipid-soluble patch. For intra-rectal application it may prove desirable to apply the instant cellular compositions, e.g. in a suppository. In other embodiments, for pulmonary airway restoration, regeneration and rejuvenation it may prove desirable to administer the instant cellular compositions by intranasal or intrabronchial instillation (e.g., as pharmaceutical compositions suitable for use in a nebulizer). For gastrointestinal regenerative medicine it may prove desirable to administer the instant cellular compositions by gastrointestinal delivery (e.g., with a capsule, gel, trouch or suppository). Also contemplated are suppositories for urethral and vaginal use in regenerative medical treatments of infertility and the like. In one embodiment, the subject pharmaceutical compositions are administered via suppository taking advantage of the migratory capacity of instant cells, e.g., migration between the cells in the epithelial lining cells in the rectum, into the interstitial tissues and into the blood stream in a timed-release type manner. Where conventional methods of administration may be ineffective in certain patients and a more continuous regenerative, restorative or rejuvenative source of therapy is desired, the instant methods, i.e., employing the instant cellular compositions make it feasible to administer therapy in a multi-dosage form, e.g. via an implantable mini-pump (such as used for delivery of insulin in patients with Type 1 insulin-dependent diabetes mellitus). Alternatively, in other cases it may desirable to deliver the instant cellular compositions over a longer period of time, e.g., by infusion.

In certain alternative embodiments, the method may involve administration of an intravenous bolus injection or perfusion of the instant cellular compositions, or may involve administration during (or after) surgery, or a prophylactic administration. In certain other embodiments, the instant administration may involve a combination therapy such as the instant cellular composition and a second drug including, but not limited to, an anti-coagulant, anti-infective or anti-hypertensive agent.

The route of delivery of the subject preparations, according to the instant methods, determined by the particular disease. For topical application it may be useful to apply the instant cellular compositions at the local site (e.g., by injection, while for other indications the preparations may be delivered by intravenous, intraperitoneal, intramuscular, subcutaneous, intranasal, and intradermal injection, as well as, by transdermal delivery (e.g., with a lipid-soluble carrier in a skin patch placed on the skin), or even by oral and/or gastrointestinal delivery (e.g., with a capsule, tablet or suppository).

In one embodiment, reprogrammed pluripotent adult somatic cells are provided. Reprogramming refers to a dedifferentiation process wherein an adult somatic cell or multipotent stem cell such as a cell committed to forming certain tissue cell lines, is exposed intracellularly to pluripotency factors, such as Oct-4 complex proteins, pluripotency factor DNAs or proteins, to yield an RC, i.e., an ESC-like pluripotent cell capable of forming any body cell.

Pluripotency factor refers to a transcription factor expressed by a pluripotent stem cell and functionally involved in inducing or maintaining the epigenetic genomic state conducive to unlimited growth and differentiation of the pluripotent stem cell; and/or, directly involved in the unlimited growth potential of the pluripotent stem cell; and/or, involved in maintaining the capacity of the pluripotent stem cell to differentiate into a cell of an ectodermal, mesodermal or endodermal lineage. Representative examples of the instant pluripotency factors include, but are not limited to, Oct-4, Sox-2, Klf-4, Nanog, c-myc, Rybp, Zfp219, Sall4, Requiem, Arid 3b, P66β, Rex-1, Nac1, Nanog, Sp1, HDAC2, NF45, Cdk1, PLZF, cRET, Stellar, VASA and EWS. In one embodiment, the pluripotency factors are isolated proteins, DNAs or RNAs. In a non-limiting example, the pluripotency factor DNAs are inserted into plasmids prior to transfer into cells. Embodiments disclosed herein provide methods for reprogramming cells in primary somatic cell cultures with pluripotent stem cell transcription factor DNAs, RNAs and proteins.

In certain embodiments disclosed herein, one or more pluripotency factors are used to reprogram cells. In another embodiment, two or more pluripotency factors are used to reprogram cells. In another embodiment the two factors are selected from the group consisting of Nanog and c-Myc, Oct-4 and c-Myc, Oct-4 and hTERT, Nanog and c-Myc and Nanog and hTERT

In another embodiment, the pluripotency factors comprise five factors are referred to as “5 transcription factor” or “5 TFactor” proteins or DNA. The 5 transcription factors are Oct-4, c-Myc, Sox-2, Klf4 and Nanog.

Embodiments disclosed herein provide RC cellular compositions that contain greater than 75% of cells expressing one or more pluripotent stem cell marker such as Oct-4, nanog, SSEA-3/4, TRA1-60 and Rex-1; preferably, greater than 80% of cells express one or more pluripotent stem cell markers; more preferably, greater than 90% of cells express one or more pluripotent stem cell markers; and, most preferably, greater than 95% of cells express one or more pluripotent stem cell markers.

Embodiments disclosed herein provide RC cellular compositions where pluripotency is confirmed by requiring that the cells have been passaged more than 10 times since their isolation; preferably, the cells have been passaged more than 12 to 14 times since isolation; more preferably, the cells have been passaged more than 15 to 16 times since isolation; and, most preferably, the cells have been passaged more than 17 to 18 times since isolation. As an additional, or alternative, proof of pluripotency, it may be required that the cells have undergone more than 20 cell division cycles since their isolation; preferably, the cells have undergone greater than 30 cell division cycles since isolation; more preferably, the cells have undergone greater than 40 cell division cycles since isolation; and, most preferably, the cells have undergone greater than 50 cell division cycles since isolation.

While illustrated in the Examples section, below, with human cells those of ordinary skill in the art will recognize that the instant disclosure of therapeutic reprogramming of human cells, enables similar cellular compositions to be developed from somatic cells of laboratory animals, domesticated and wild animals, birds and boney fishes.

The instant RC cellular compositions are precursors in production of differentiated tissue cells (DTC) such as adipocytes, chondrocytes, neural cells, epithelial cells, muscle cells, cardiomyocytes, pancreatic islet cells, osteocytes, lung parenchymal cells, liver hepatocytes and renal epithelial and proximal tubule cells. Embodiments disclosed herein provide methods for producing DTC compositions such as, but not limited to, by culturing the instant cellular compositions under defined conditions in a differentiation media that is suitable and sufficient for the induction and growth of specific different types of DTCs. Several representative examples are provided, by way of illustration, in the Examples section. That an instant cellular composition has differentiated into a DTC may be determined by testing the staining reaction of the cells or testing for the presence of a cell surface marker or an RT-PCR marker. Representative examples of staining tests for determining that a instant cellular composition has differentiated into a DTC include Oil Red O staining for adipocytes, Alcian Blue staining for chondrocytes and Alizarin Red S staining for osteocytes. Representative examples of cell surface markers for determining that a reprogrammed cell according to the invention has differentiated into a DTC include tub-III, Map2, Nestin, O4, GalC and GFAP for certain neural cells; tub-III, Map2, Nestin, O4, GalC and GFAP for other types of neural cells; and, troponin, connexin 43 and cardiac-actin for cardiomyocytes.

In other embodiments, the invention provides methods for autologous cell therapy including, but not limited to, a process where a practitioner collects adult somatic cells from a subject; a laboratory or a machine therapeutically reprograms the cells in the sample ex vivo to product RC; and, the cells are then administered therapeutically to the same subject. Autologous RC do not express “foreign” histocompatibility antigens; are recognized as “self” by the immune system of the subject; are not subject to transplant rejection; and, do not mediate graft versus host disease (GVHD). Such advantageous properties make the instant RC the cells of first choice for patient therapies.

In summary, the present therapeutic reprogrammed pluripotent adult somatic cells with ESC-like cell have plasticity and may be used as a cellular replacement therapy in different disease/trauma states including e.g. treatments of Parkinson's disease, multiple sclerosis, amyotrophic lateral sclerosis (ALS), Alzheimer's disease, cystic fibrosis, fibromyalgia, Type-1 diabetes, non-union bone fractures, cosmetic and reconstructive surgery for skin, cartilage and bone, myocardial infarct, stroke, spinal cord injury, traumatic injury, and restoring, regenerating and rejuvenating damaged and aged tissues.

### EXAMPLES

To successfully induce therapeutic reprogramming in adult somatic cells, pluripotent factors (present in embryonic cells) need to be introduced into cells so that they can act intracellularly. Other investigators have recently expressed certain factors in fibroblasts cells using viral transduction, but these methods have the disadvantage that they cannot be transferred to uses in human therapies. Mindful that cellular proteases in proteosomes rapidly degrade endocytosed proteins and that significant quantities of intracellular protein might be required to achieve reprogramming, a test system was developed for insuring that cells received appropriate levels of reprogramming instructions, and a delivery method was developed that bypassed endocytic and phagocytic pathways leading to proteosomes.

Material and Methods:

Cells:

Human foreskin fibroblasts (HFF) were obtained from the American Type Culture Collection (ATCC; ATCC#120707). Human embryonic fibroblasts (HEF) were isolated from human umbilical cord tissue using collagenase and proteases. Human embryonic kidney (HEK) cells were obtained from the ATCC. Human fetal retinal pigment epithelial cells (RPE) were isolated from human fetal retinal tissues using collagenase and proteases. Human testicular cells were isolated using collagenase and protease, i.e., methods described in co-pending U.S. patent application Ser. No. 11/488,362 (filed Jul. 17, 2006), Ser. No. 11/423,676 (filed Jun. 12, 2006) and Ser. No. 11/694,687 (filed Mar. 30, 2007), incorporated herein by reference in their entirety. HT42 cells are fibroblastic cells from adult human testicular tissue, recovered after enzymatic digestion and selection for adherent cells.

Purification, Cutting and Oxidation of SWNTs.

SWNTs (20 mg) grown by laser ablation were mixed with 100 mL of 2.5 M HNO₃, refluxed for about 36 hr, sonicated with a cup-horn sonicator (Branson Sonifer 450) for 30 min to cut the nanotubes into short segments and refluxed again for another 36 hr. After this treatment, the mixture was filtered through a polycarbonate filter (Whatman, pore size 100 nm), rinsed thoroughly and then re-suspended in pure water by sonication. The aqueous suspension was then centrifuged at 7,000 rpm for about 5 min to remove any large impurities from the solution. SWNTs after these processing steps were in the form of short (tens to hundreds nanometers) individual tubes (about 1.5 nm in diameter) or small bundles (up to 5 nm in diameter) and re-suspended to give a concentration of about 0.04-0.05 mg/mL. Acidic oxygen groups (e.g., —COOH) on the sidewalls of the tubes rendered solubility or high suspension stability of the SWNTs in water and buffer solutions.

Transcription Factor Proteins.

Recombinant transcription factor proteins were prepared in E. coli by standard molecular genetic methods involving introduction of nucleotide sequences encoding Oct-4, Sox-2, Klf-4, Nanog and c-Myc into pGEX expression vectors, and the recombinant proteins were purified from bacterial lysates. The proteins were conjugated to SWNTs by the following method. A suspension of the oxidized and cut SWNTs at a concentration of about 0.05 mg/mL was mixed with fluorescently labeled proteins (typical protein concentration about 1 μM) for about 2 hr at room temperature prior to characterization (by atomic force microscopy (AFM) for imaging protein-SWNT conjugates) or cellular incubation for uptake. After this mixing step, proteins were found to adsorb non-specifically onto nanotube sidewalls.

iPS Induction:

Lentivirus production was performed as described earlier (Ramezani et al, Curr Prot Mol Biol, 2002). Retinal pigment epithelial cells (RPE) and human embryonic fibroblasts (HEF) were infected with lentiviral particles containing the cDNA of Oct-4, Sox2, KLF4, c-Myc and Nanog at an approximate MOI of 10. Infected cells were grown under normal culture conditions in untreated dishes for 6 days and subsequently seeded onto mouse embryonic fibroblasts (MEF) feeder cells in hESC medium at a density of 5×10⁴/10 cm dish. Colonies were picked and clonally expanded with passaging every 3-7 days onto fresh MEF feeder cells by either trypsinization (RPE) or manual picking.

Cell Surface Marker Staining:

Rabbit-anti-human Tra1-60, Tra1-81 and SSEA4 were incubated with live cells for 1 hr in normal growth medium followed by washing and detection of the primary antibody with a secondary TRITC-labelled sheep-anti-rabbit antibody.

Gene Expression:

A 25 gene XP-PCR multiplex was performed according to the manufacturer's instructions (Beckman, GeXP start kit) and analyzed in the Genome Lab GeXP capillary electrophoresis instrument (Beckman). Gene expression data was evaluated on a standard hESC RNA curve according to the manufacturer's instructions. PCR primers were constructed for the cDNAs of Oct-4, Sox2, Nanog, KLF4 and c-Myc and the 3′ UTR of the same mRNAs to distinguish between endogenous plus exogenous expression (cDNA) and endogenous expression only (3′ UTR). Additionally, the following gene expression patterns were also analyzed: Lin28, Col5A2, mouse GAPDH (to test for feeder layer contamination), human GAPDH, cRET, Brachyury, TERT, Thy1, Rex1, Dppa5, ALPL, beta Actin, Sall4 and Cripto (TDGF1).

### Example 1

### The HT NP-RFP/OP-GFP Cell Line

Effects of potential reprogramming factors and treatments can be difficult to assess in tissue culture. ESC-like morphological changes can take days or weeks to manifest themselves and are not always indicative of pluripotency. An unbiased cellular reporter system was constructed based on the assumptions that: (i) transcription factor Oct-4 is key to inducing down-stream gene expression on the pathway of therapeutic reprogramming to an ESC-like state in adult cells; (ii) Nanog activation by Oct-4 is a most important key to inducing this adult cell therapeutic reprogramming; (iii) exogenous Oct-4 is likely not sufficient to induce therapeutic reprogramming without activation of endogenous Oct-4 expression; and, (iv) the optimal effects of these two transcription factors are most easily viewed in the context of a receptive cellular genetic and epigenetic background, i.e., as present in human testicular cells (HT; as disclosed in PCT/US2006/004077, filed Feb. 16, 2005 and published as WO2006/084229, incorporated herein by reference in its entirety).

A stable reporter cell line for monitoring therapeutic reprogramming was constructed wherein the Nanog promoter (NP) was used to drive expression of red fluorescent protein (RFP) and the Oct-4 promoter was used to drive the green fluorescent protein (GFP). Furthermore, a construct which stably and constituently expresses GFP only was used as a control for transduction efficiency. Theoretically, Oct-4 activation of the Nanog promoter would cause the single cells to exhibit red fluorescence; cell surface staining with FITC-tagged fluorescent antibodies specific for stem cells, i.e., like anti-SSEA-4, would result in a green color; and, agents activating both the NP and staining positive for cell surface markers result in yellow color (red color plus green color causing a summation to a yellow coloration) if the cells were associated.

The HT NP-RFP or OP-GFP reporter cells were constructed as follows: a replication defective lentiviral vector construct (LentiMax™) containing either the NP-RFP or the OP-GFP were manufactured by Lentigen Corporation (Baltimore, Md.) and were independently transfected into 293FT producer cell lines at 37° C. in 95% air/5% CO₂ for 4 hr. After 48 hr of productive viral vector infection, the resultant replication defective, infectious lentiviral particles were concentrated and quantitative PCR for the gag region was used to determine viral titer. The HT-40 cell line sample was sorted by FACS (fluorescence activated cell sorting) to derive cKit(−), Thy(+) and α-integrin(+) cells. For viral transduction, 1×10⁵ HT-40 cells were incubated with 1×10⁷ NP-RFP lentiviral particles; and, in parallel, 1×10⁵ HT-40 cells were incubated with 1×10⁷ GFP lentiviral particles. The conditions of incubation were as follows: transduction of cells was in 1 mL of serum-free PM10 supplemented with growth factors, (disclosed in PCT/US2006/028043; filed Jul. 17, 2006; incorporated herein by reference in its entirety), containing 4 μM protamine sulfate (Calbiochem) and in 24 well dishes that had been coated with 0.1% gelatin to provide substrata for cell adherence. Immediately after all of the components were added to each well, the plate was centrifuged at 1,400×g for 60 min at 21° C. to distribute the cells onto the substrata and allow for more efficient transduction, the tissue culture medium was then removed and replaced with PM10 supplemented with growth factors and 10% FBS. The plates were incubated at 37° C. overnight and for the next 14 days the medium was changed every 3 days with PM 10 supplemented with growth factors and 10% FBS until the wells became confluent and could passaged and maintained in PM10 supplemented with growth factors and 10% FBS and adherent on 0.1% gelatin coated dishes.

Evidence for lentiviral transduction was provided by the control GFP. Cells transduced with this lentiviral construct exhibited green fluorescence within 1-2 days following transduction.

HT cells express low levels of Nanog, allowing endogenous low-level expression to provide a proof of principle that the HT NP-RFP lentiviral transduced cells were indeed functional. Within 24 hr after lentiviral transduction of NP-RFP, the first faint red cells were observed. Evidence presented in Example 5, confirmed that these reporter cells were functional and able targets for therapeutic reprogramming.

### Example 2

### Single Wall Nanotube-Mediated Delivery

The ability of very small carbon single wall nanotubes (SWNT) to deliver large proteins into cells was tested to determine whether, if sufficiently small, the SWNT might bypass endocytic routes of entrance and, if appropriately charged, they might adhere to proteins.

In order to “clear” the SWNT (Lythmus Nanotechnology), a SWNT solution at 2 mg/ml was first autoclaved in a liquid cycle for 30 min and then centrifuged at 6,000 rpm in a microcentrifuge for 5 min to remove clumps and debris. The “cleared” supernatant was used for subsequent experiments.

The potential ability of SWNT to deliver large proteins into cells via a non-endosomal and endosomal penetration was tested as follows:

1. 1 μg/mL of cleared nanotubes were mixed with 1 μg/mL IgG labeled with GFP green fluorescent protein;

2. To allow for coating of the nanotubes with the IgG-GFP test protein, the suspension was incubated for 2 hr at 4° C.;

3. The suspension was examined under fluorescent microscopy to determine whether GFP was adherent to the SWNT. Under compound light microscopy clumps of grey SWNT were easily observed and when observed using fluorescence microscopy the clumps, as well as individual particles, were stained green, which indicated that they were coated with adherent GFP;

4. Next, human embryonic fibroblasts (HEF; 3×10⁵ in 100 μL D-MEM) were incubated in suspension with 1 μL of the GFP-coated SWNT at 4° C. (to discourage endosomal entry) and in the presence of 200 μM chloroquine (an inhibitor of endocytosis and lysosomal fusion with endosomes);

5. After 90-120 min at 4° C. the cells were collected by centrifugation and washed 3 times to remove unbound GFP-SWNT;

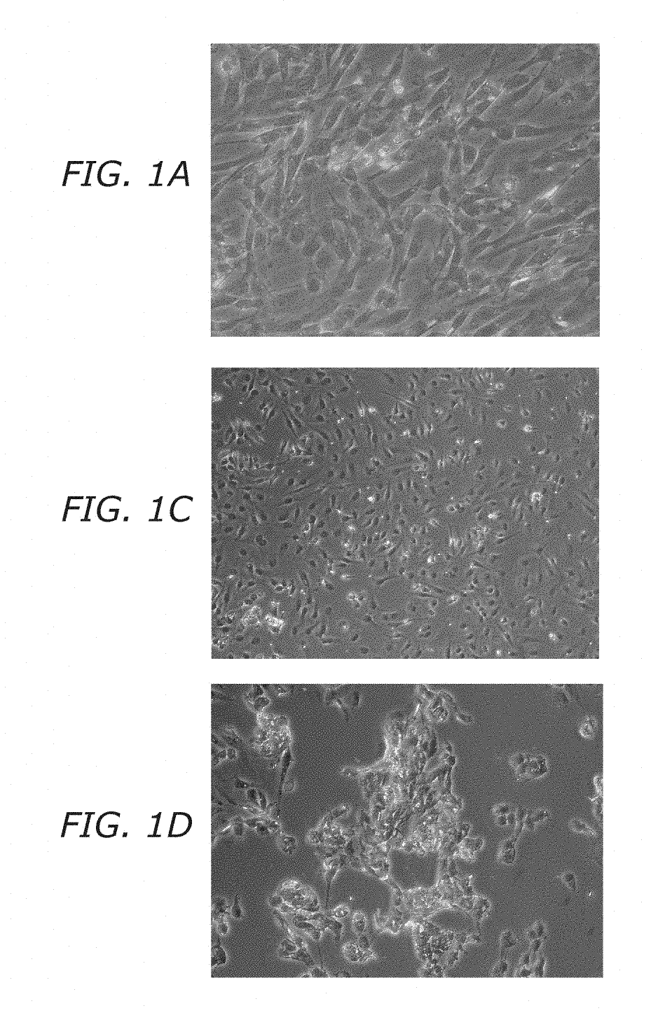

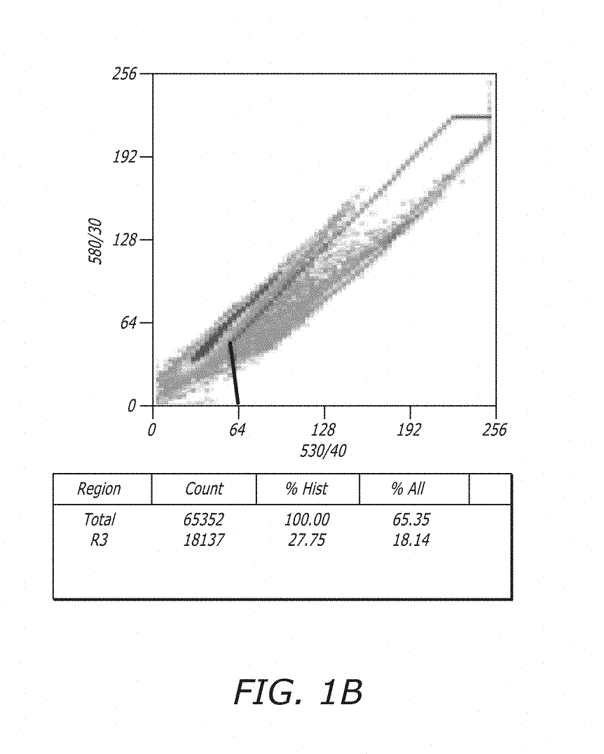

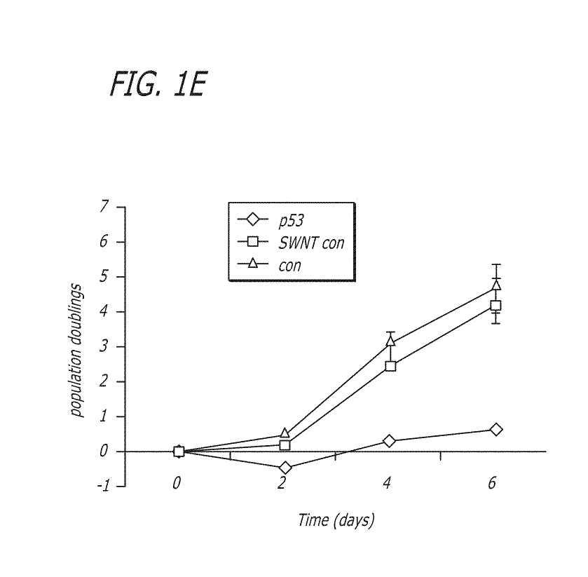

6. When examined using fluorescence microscopy the cytoplasm of the cells was stained green in a patchy pattern, but the periphery of the cell (cell membrane) was clearly demarcated and relatively devoid of staining (FIG. 1A); and

7. When the SWNT-Oct-4 protein-treated cells were incubated at 37° C. overnight (10-16 hr) in D-MEM supplemented with 10% FBS the fluorescent green staining pattern was perinuclear (surrounding the nucleus).

Control suspensions of cells treated with either nanotubes alone or GFP alone at 4° C. under identical conditions did not show cytoplasmic or perinuclear staining.

FIG. 1A depicts GFP transduced into HEF 885 by SWNT as evidenced by green cytoplasmic fluorescence in a suspension of cells 5 hr after SWNT-mediated transduction of IgG-GFP. SWNT were coated with Alexa488 IgG-GFP under the following conditions: 100 μL of a 2 mg/mL solution of IgG-GFP was added into a suspension of SWNT consisting of 33 ng/2 ml water and coating of the SWNT was for 2 hr. The fluorescent photoimage was at 40× magnification at 24 hr after SWNT transduction.

FIG. 1B shows a FACS analysis of SWNT IgG-GFP with a transfection efficiency of 81%. IgG-GFP was bound onto SWNT and delivered into HEFs as discussed earlier. Twenty-four hours post transfection, cells were taken off the Petri dish and analyzed for GFP.

Following the same SWNT binding protocol as above, p53 protein was bound to SWNT and delivered into HeLa cells and p53 knock-out fibroblasts. P53 is one of the main proteins responsible for inducing apoptosis and growth inhibition in cells. Therefore, if delivered into the cell, the cell should stop growing and undergo apoptosis. As expected, HeLa and p53 knock-out fibroblasts stopped growing and underwent apoptosis (FIGS. 1C, 1D and 1E). FIG. 1C shows a photoimage of HeLa cells before treatment. FIG. 1D shows a photoimage of HeLa cells 48 hr after treatment with p53 bound to SWNT. This image includes dying cells and overall inferior cell morphology as compared with FIG. 1C. The cells have been stained with an antibody against p53 to visualize remaining p53. FIG. 1E shows a growth curve assay of p53 knock-out MEF untreated, SWNT only treated and p53-SWNT treated. There was a steep decline in cell number and growth rate of p53-SWNT treated MEFs as compared to control and control-SWNT. This implies that the delivered p53 protein induced cell death and growth inhibition.

The latter findings are highly suggestive that (a) SWNT transported Alexa 488 bound IgG into cells via a process that took place at 4° C. an in the presence of chloroquine by showing that it minimally involved endosomes and/or phagolysosomes; (b) the distribution of Alexa 488-SWNT fluorescence in the cytoplasm without evidence of cell membrane staining is supportive of an intracellular localization of GFP; (c) the final perinuclear distribution of staining into Golgi and endoplasmic reticulum and not into phagolysosomes further supports a localization of Alexa 488-SWNT; and (d) the finding that green fluorescence was still observed in the cytoplasm after overnight culture at 37° C. shows that the GFP was not degraded confirming a probable non-phagolysosomal intracellular localization. Furthermore, the delivery of a known apoptosis and growth inhibitor protein showed severe induction of cell death and growth inhibition in at least two cell types, strongly supporting the notion that a large, biologically active protein can be delivered into cells using SWNTs and that it stays functional. Even after 48 hr inside the cell, the p53 protein was still detectable by immunofluorescence, again confirming the stability of proteins over this period of time if delivered using SWNTs.

### Example 3

### Immunoprecipitation of the Oct-4 Complex

Oct-4 is a transcription factor strongly expressed in ESC and these cells are presently the benchmark cell type for pluripotency. To test the effects of Oct-4, and the proteins associated with it, in somatic cells, the Oct-4 complex was immunoprecipitated from ESC extracts as follows:

1. Two 6 well plates of growing human ES cells (Invitrogen) were washed twice in PBS and scraped into 500 μl of RIPA buffer (50 mM Tris/HCl, 150 mM NaCl, 1 mM ETA, 1% TritonX 100, 1 mM PMSF, Protease Inhibitor Cocktail (Sigma));

2. The lysate was frozen at −80° C. and immediately afterwards thawed at 37° C. and the freeze-thaw procedure was repeated twice;

3. The cell debris were pelleted at 17,900×g and the cell lysate supernatant was used in the following steps;

4. To the cell lysate, 20 μL of rabbit-anti-Oct-4 antibody (Santa Cruz) was added and incubated for 45 min on ice;

5. To precipitate the antibody-Oct-4 complex, 40 μL of a 50% slurry of Protein A/G sepharose beads (Invitrogen) was added to the lysate and the suspension was incubated for 45 min on ice;

6. The beads were pelleted at 1,000×g, the supernatant was discarded and the beads were washed 2 times with PBS containing 0.05% Triton X-100 (Sigma) to remove non-specifically adsorbed proteins;

7. To elute bound proteins from the Oct-4 immunoprecipitate, 50 μL of 2M NaCl, 10 mM NaCitrate, pH 3 was added and the beads were vortexed and incubated for 30 min at 21° C.;

8. Eluted proteins were collected by removing the beads at 1,000×g and collecting the supernatant into a hypodermic needle; and

9. To neutralize the low pH 3 buffer, the supernatant was diluted to a final salt concentration of 200 mM in water (GIBCO).

### Example 4

### Coating SWNT with Proteins of the Oct-4 Complex

To the Oct-4-containing eluate of Example 3, 50 μl of “cleared” (Example 2) SWNT were added. After incubating the suspension for 2 hr on ice, the coated SWNT were collected by centrifugation at 17,900×g and washed once with PBS. SWNT coated with the Oct-4-immunoprecipitated proteins were resuspended in 2 mL of PBS. They are either used immediately or frozen and stored at −80° C. until use.

### Example 5

### Treating HT NP-RFP Cells with SWNT Coated with the Oct-4 Complex

The results presented in Example 2 showed SWNT to be capable of transporting GFP into cells via a non-phagosomal pathway and with minimal protein degradation. To evaluate the biological activity of cell penetrable Oct-4 complex proteins, 50 μl of the Oct-4 protein coated SWNT (Example 4) was added to a suspension of 3×10⁵ reporter cells prepared in Example 1. After incubation for 3 hr at 4° C., cells were collected by centrifugation at 1,000 rpm in a microcentrifuge and the resultant cellular pellet was resuspended in 2 mL of PM10 medium (disclosed in PCT/US2006/028043; filed Jul. 17, 2006; incorporated herein by reference in its entirety) supplemented with twice the normal levels of the growth factors disclosed therein. The treated cells were plated into 1 well of a 6 well plate. After overnight culture (10-16 hr) at 37° C., cells were examined for expression of the NP-RFP reporter using fluorescence microscopy and the cells continuously monitored for the next 21 days.

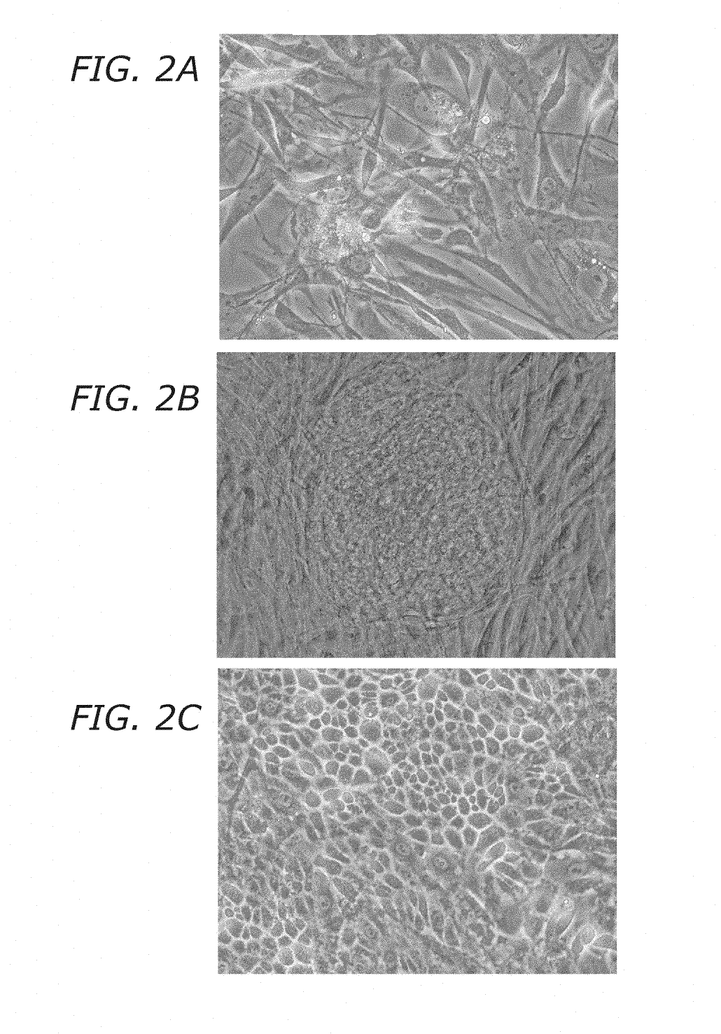

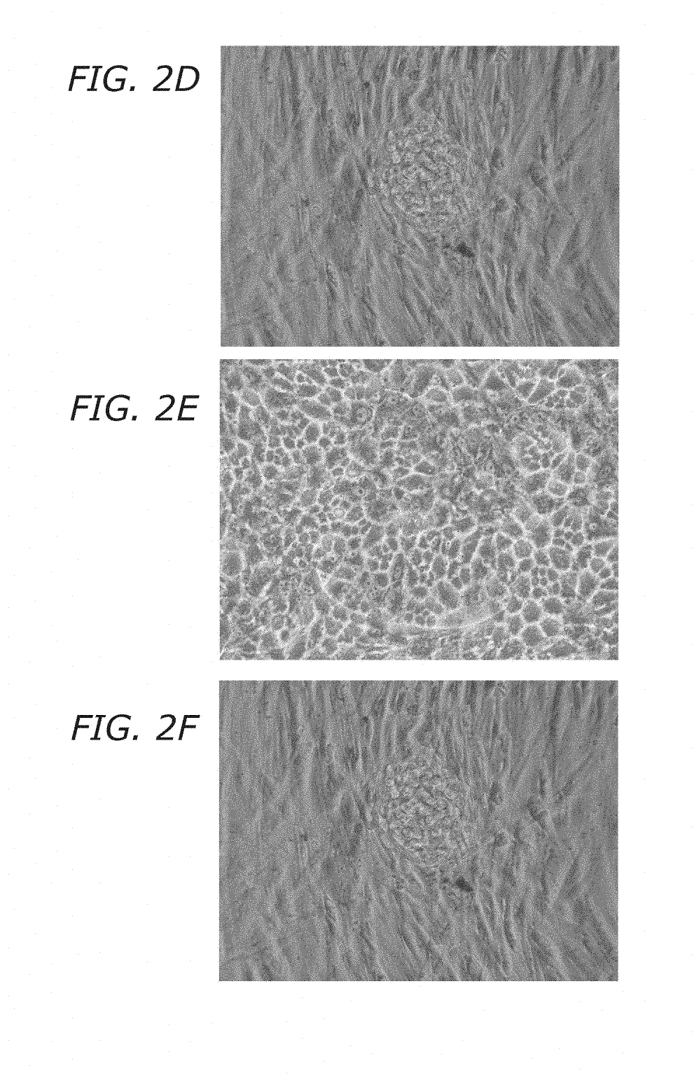

FIG. 2A is a photoimage of the HT42 NP-RFP reporter cells before transduction. FIG. 2B depicts Oct-4 complex-protein-SWNT transduced HT40 NP-RFP reporter cells wherein Nanog expression was upregulated leading to expression of red fluorescent protein (RFP), a suspension of HT40 NP-RFP reporter cells 11 days, after treatment with the Oct-4-immunoprecipiate-coated-SWNT (3 hr coating of the SWNT). The suspension was cultured in PM10 medium supplemented with 2× growth factors and 10% FBS. The fluorescent photoimage was obtained at 16× magnification.

About 5% of the cells in these cultures were present as bright red cells (FIG. 2B) indicating (a) successful cellular penetration of SWNT-Oct-4 protein; (b) retention of biological activity in the cell penetrant Oct-4 proteins; and, (c) successful activation of the Nanog promoter driving expression of RFP in at least 5% of the cells in the cell cultures. Surprisingly, within 48 hr the red-stained cells changed morphology and clumps of red cells began to appear, similar to the clumps in which ESC grow. The Oct-4 protein-SWNT-transduced cell cultures were maintained at 37° C. and after 14 days about 5 to 10 individual colonies had been established as independent continuous cell cultures wherein each had expanded to include about 50 to 100 cells. Each of these cell cultures constitute a stable continuous independent RC line. Furthermore, ESC lysate, from where the Oct-4 complex proteins were obtained, was also bound to SWNT and delivered into different cell lines. As seen with the isolated Oct-4 complex proteins, ESC lysate-SWNT induced colonies and, in HT42 NP-RFP cells, expressed Nanog as detected by the presence of RFP driven by the Nanog promoter.

FIG. 2C depicts a colony of retinal pigment epithelial cells 14 days post transfection with Oct-4 complex proteins bound to SWNT. FIG. 2D shows a colony of HFF cells 14 days post transfection with Oct-4 complex proteins bound to SWNT. FIG. 2E shows a colony of HT42 NP-RFP cells 14 days post transfection with ESC lysate proteins bound to SWNT. RFP was detected using a fluorescence microscope and filtering for red fluorescence. RFP expression confirms the expression of Nanog since RFP is expressed from the Nanog promoter locus in these cells. FIG. 2F shows a colony of RPE cells 14 days post transfection with ESC lysate proteins bound to SWNT.

These experiments prove the possibility to deliver biologically functional proteins derived from ESC lysate into a variety (HT42 NP-RFP, HFF, RPE) of cells and elicit a change in morphological growth pattern and gene expression. The observed changes are in line with a change toward a stem cell-like state since the expression of Nanog combined with the colony growth pattern define a stem cell-like state.

### Example 6

### RC can Differentiate into Mesodermal Tissues

The ability to differentiate into osteogenic, chondrogenic, and adipogenic cell types is a well established hallmark of ESC.

In order to induce adipogenic differentiation, RC are plated onto 0.2% gelatin (Sigma) coated 4-well plates (VWR, Brisbane, Calif.) at 20,000 cells/cm² in hMSC Adipogenic Differentiation BulletKit (ADB) prepared according to manufacturer protocol (Cambrex, East Rutherford, N.J.)+5% FBS (Hyclone). Cells are maintained in ADB for 7 days, adipogenic maintenance media (AM) ((DMEM-LG/GL (Invitrogen, Carlsbad, Calif.)+1% penicillin/streptomycin (Invitrogen)+15% FBS (Hyclone)+10 μM insulin (Sigma)) for 3-4 days, and then interchanged from AM to ADB every 3-4 days for approximately 20 days total.

In order to induce osteogenic differentiation, RSC are plated in the same manner as for adipogenic differentiation and treated with hMSC Osteogenic Differentiation BulletKit (ODB) prepared according to manufacturers protocol (Cambrex)+5% FBS and were in ODB with 50% media changes every 3-4 days for approximately 20 days total.

In order to induce chondrogenic differentiation, RC are plated onto 0.2% gelatin coated 6-well plates (VWR) at approximately 6,000 cells/cm² in hMSC Chondrogenic Differentiation BulletKit (CDB) prepared according to manufacturer protocol (Cambrex)+1% FBS+20 ng/ml TGF-β3 (R&D Systems, Inc., Minneapolis, Minn.) added fresh. Cells receive full media changes every 3-4 days for approximately 14-20 days.

All cells are fixed in 4% paraformaldehyde (Electron Microscopy Science, Hatfield, Pa.) for 10 minutes at room temperature (RT). Adipogenic induced cells are stained for fat vacuoles using the oil red O staining kit (American Master Tech Scientific, Lodi, Calif.). Briefly, cells are washed with 70% ethanol (EMD Chemicals Inc., San Diego, Calif.), incubated for 10 minutes RT with oil red O, and counterstained with Modified Mayer's Hematoxylin (MMH) (American Master Tech Scientific). Osteogenic induced cells are stained for calcium deposits using alizarin red S (Fisher Scientific, Pittsburgh, Pa.). Briefly, cells are washed 2× with water, incubated 1 hour at RT with 0.0075% alizarin red S (Fisher Scientific) diluted in dH₂O, and counterstained with MMH. Chondrogenic induced cells are stained for sulfated proteoglycans using alcian blue (Sigma). Briefly, cells are incubated with 1% alcian blue in 0.1N HCL for 1 hour RT, washed 1× with 0.1N HCL for 5 minutes RT, and counterstained with MMH. Pluripotent marker antibodies used are: Oct-4 (Santa Cruz Biotech, Santa Cruz, Calif.), Nanog (Cosmo Bio, Carlsbad, Calif.), Thy-1, and SSEA-4 (Chemicon). Visualization is achieved using the following secondary antibodies in combinations or separately: Alexa Fluor 488 anti-mouse, Alexa Fluor 488 anti-rabbit, Alexa Fluor 568 anti-rabbit, Alexa Fluor 568 anti-mouse, (all from Invitrogen), biotinylated anti-rabbit IgG and fluorescein-streptavidin (Vector Laboratories, Burlingame, Calif.). Nuclei are stained using DAPI (Invitrogen). Slides stained with fluorescence were analyzed using an Olympus BX-61 microscope with SlideBook image software while mesodermal staining is analyzed using a Leica DM IRB microscope with Microsuite Biological suite imaging software.

RSC induced to the osteogenic lineage, chondrogenic lineage, and adipogenic lineage all display histological characteristics of each cell lineage as compared to control non-induced RC; calcium deposits using alizarin red S staining typical of bone, sulfated proteoglycans using alcian blue staining for cartilage, and Oil Red O staining for fat vacuoles. Such data demonstrates that RC easily differentiate into mesodermal tissues.

### Example 7

### RC can Differentiate into Neurogenic Tissues

In order to induce neural differentiation, cells are plated onto fibronectin-coated cover slips (BD Biosciences, San Jose, Calif.) in 6-well plates at approximately 20,000 cells/cm² in Neuronal Induction Media (NIM) consisting of DMEM-F/12 (Invitrogen)+1% penicillin/streptomycin (Invitrogen)+1×N-2 supplement (Invitrogen) plus the following different conditions: 1) 10 ng/ml fibroblast growth factor (FGF) for 2 days, passed ˜1:3 in 10 ng/ml FGF+10 ng/ml platelet derived growth factor (PDGF)+20 ng/ml epidermal growth factor (EGF) for 5 days, and 10 ng/ml FGF+10 ng/ml PDGF excluding EGF for 8 days totaling 15 days. 2) 100 ng/ml FGF for 5 days, passed ˜1:3 in 100 ng/ml FGF for 2 days, 200 ng/ml sonic hedgehog (SHH)+100 ng/ml fibroblast growth factor 8 (FGF8) for 8 days, 200 μM ascorbic acid (AA)+10 ng/ml glial cell line-derived neurotrophic factor (GDNF)+20 ng/ml brain-derived neurotrophic factor (BDNF) for 7 days totaling 22 days. 3) 100 ng/ml FGF, after 24 hour attachment 10 uM retinoic acid (RA) (Sigma) for 5 days. After 9 days total in 100 ng/ml FGF cells are passed ˜1:3 into the following two conditions: a) 10 ng/ml FGF+20 ng/ml nerve growth factor (NGF)+20 ng/ml BDNF or b) 10 ng/ml FGF+20 ng/ml NGF+10 ng/ml GDNF for 16 days each. Next 200 μM AA+10 ng/ml GDNF+20 ng/ml BDNF is added for 27 days totaling 58 days. All cells are continually cultured on fibronectin-coated cover slips and full media changes occurred every 2-3 days with fresh growth factors (all from R&D Systems, Inc.).

The cells were fixed, stained and analyzed as described in Example 6.

In order to demonstrate the plasticity of RSC, cells are differentiated toward a neural lineage and assessed their cellular and molecular marker expression. When RSC are placed into media containing FGF and retinoic acid for 5 days, followed by the addition of GDNF, NGF, FGF for 16 days, then the addition of ascorbic acid, GDNF, and BDNF for 27 days the cells expressed oligodendroglial markers O4 and GalC. When BDNF is substituted for GDNF in the second step above, cells obtain a neuronal morphology and expressed the neuronal lineage marker tub-III. When cells are placed into FGF, FGF8, and SHH followed by ascorbic acid, GDNF, and BDNF cells express the mature neuronal marker Map-2 and the neural progenitor marker Nestin. RT-PCR data can support the immunocytochemistry data by confirming that RC express neural markers at the RNA level. This data can clearly demonstrate the plasticity of RC and the potential for differentiated into multiple neural cell types.

### Example 8

### RC Differentiate into Cardiogenic Tissues

In order to induce cardiac differentiation, RC are plated onto 0.2% gelatin coated cover slips in 4-well plates at 15,000 cells/cm² in PM10+1% FBS. After 24 hr, media is changed to PM10 without growth factors (GF) minus beta-mercaptoethanol with either 2 μM or 8 μM 5-Aza-2′-deoxycytidine (Aza) (Sigma). Full media changes occurred every 2-3 days with fresh Aza treatment for 16 days.

The cells were fixed, stained and analyzed as described in Example 6.

RSC can be differentiated into cells of the cardiac lineage. Immunocytochemistry staining demonstrates positive staining for the cardiac markers actin, troponin, and connexin43 when the cells are differentiated using either 2 μM or 8 μM Aza. RT-PCR data can support immunocytochemistry findings by demonstrating that differentiated RSC express cardiac markers at the RNA level. This data can be used to confirm that RSC express cardiogenic markers at the cellular and molecular level.

### Example 9

### Oct-4 Complex: Reprogramming Factors

Components of the Oct-4 complex described in Example 3 are determined by MALDI mass spectrometry which include therapeutic reprogramming factors and accessory factors promoting reprogramming, which are as follows: namely, Rybp, Zfp219, Sa114, Requiem, Arid 3b, P66β, Rex-1, Nac1, Nanog, Sp1, HDAC2, NF45, Cdk1 and EWS. Two or more of these proteins when introduced into cells in combination are effective to induce therapeutic reprogramming of adult human cells.

### Example 10

### Reprogramming with Pluripotent Stem Cell Transcription Factor DNAs

Fibroblasts are easily extracted from skin biopsy samples obtained such as using a dermal punch. Therapeutic reprogramming of primary cultures of dermal fibroblasts was accomplished using SWNTs to which DNAs were chemically coupled using carbodiimide (EDC), through reactive carboxyl groups on the SWNT and reactive amine and amide radicals in the DNA (Example 2).

In another method, polyethylenimine (PEI) particles were bound with 5 transcription factor (5 TFactor) plasmid DNAs (Oct-4, Nanog, c-Myc, Klf-4 and Sox-2). PEI particles are a powerful transfection reagent that ensures effective and reproducible transfection with low toxicity. PEI is a purified polyethylenimine that provides effective and reproducible transfection with low toxicity. PEI is a linear polyethylenimine which compacts DNA into positively charged particles capable of interacting with anionic proteoglycans at the cell surface and entering cells by endocytosis. PEI also possesses the unique property of acting as a “proton sponge” that buffers the endosomal pH and protects DNA from degradation. The continuous proton influx also induces endosome osmotic swelling and rupture which provides an escape mechanism for DNA particles to the cytoplasm. PEI can effectively delivery DNA to various established cell lines as well as primary cells

To bind DNA onto SWNTs, the following method was used: Thirty milligrams 1-ethyl-3-(3-dimethylaminopropyl) carbodiimide hydrochloride (EDC) and 50 mg N-hydroxysulfosuccinimide (NHS) were measured separately and aliquoted as powders into 15 ml conical tubes. SWNT (300 μl) from stock solution (3.333 mg/ml) was centrifuged at 3,000×g for 90 min. Two hundred microliters of SWNT supernatant was carefully removed and mixed with 250 μl cell culture water and 50 μl sterile 1M 2-mesitylenesulfonyl chloride (MES, pH 5.38) and 120 μl 5N NaOH (pH 4.7-6.0). The SWNT solution was then triturated with an insulin needle for 30-60 seconds, the mixed with the EDC powder, vortexed for 30 seconds, and incubated at room temperature (RT) in the dark for 15 min and then centrifuged at 20,000×g for 15 min. The supernatant is removed carefully with a pipet and the SWNT pellet resuspended in 500 μl cell culture water and triturated with an insulin needle for 30-60 seconds. The SWNT solution was then mixed with the NHS powder, vortexed, and incubated at RT in the dark for 15 min, centrifuged at 20,000×g for 15 min and the supernatant carefully removed. The SWNT pellet was resuspended in 500 μl cell culture water and again triturated with an insulin needle for 30-60 seconds. The SWNT solution was then aliquoted into 5 tubes with 100 μl in each tube. Five micrograms of each 5 TFactor DNA was added into each tube, the solutions mixed well and incubated in the dark at RT for 2 hr with occasional agitation. After incubation the contents of all five tubes are combined all tubes and the contents centrifuged at 20,000×g for 12 min. The supernatant was removed and the pellet resuspended in 1 mL PBS and triturated with an insulin needle for 30-60 seconds. Plated (10 cm culture dishes) human foreskin fibroblast (HFF) cells were washed twice with PBS and once with 150 μM DMEM+cloroquine (CQ), the medium aspirated and 1-2 ml of 150 μM DMEM+CQ added to cover the cells. The SWNT-bound DNA (250 μl) was then added evenly across the plate. Then, 250 μl of 300 μM DMEM+CQ was added. If needed, an additional 1 mL 150 uM DMEM+CQ was added to fully cover the plate. The cells are then incubated for 2 hr at 37° C. in a 5% CO₂ incubator. After 2-12 hr the cells were trypsinized, washed 2-3× with PBS during collection and centrifuged at 400×g for 7 min, counted and plated at 1×10⁵ per plate in a 10 cm mitomycin C-treated MEF plate and cultured in hESC media at 37° C. in a 5% CO₂ incubator.

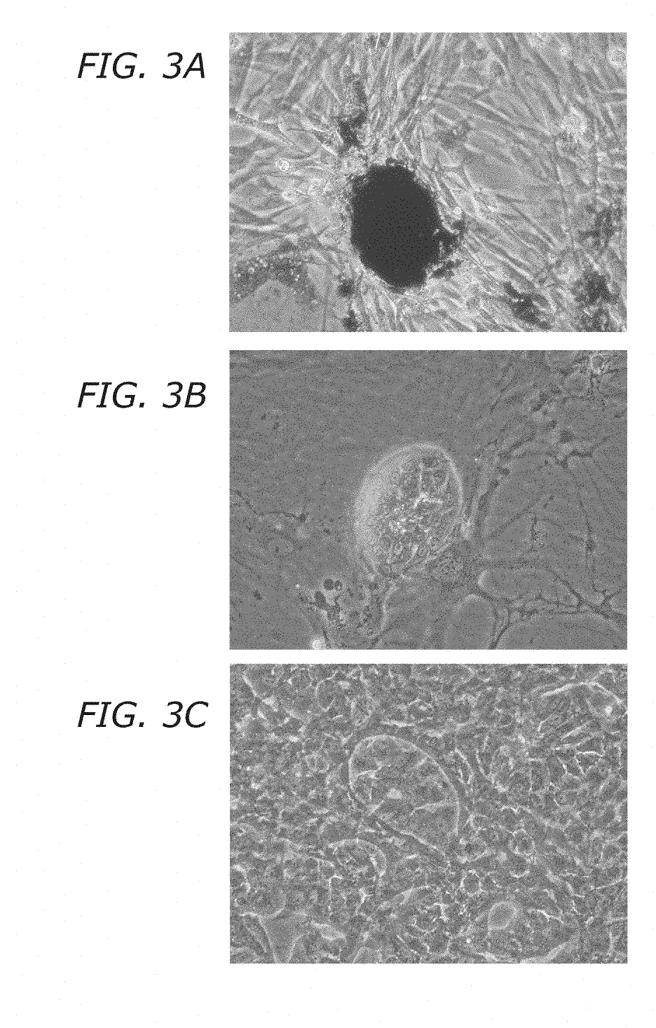

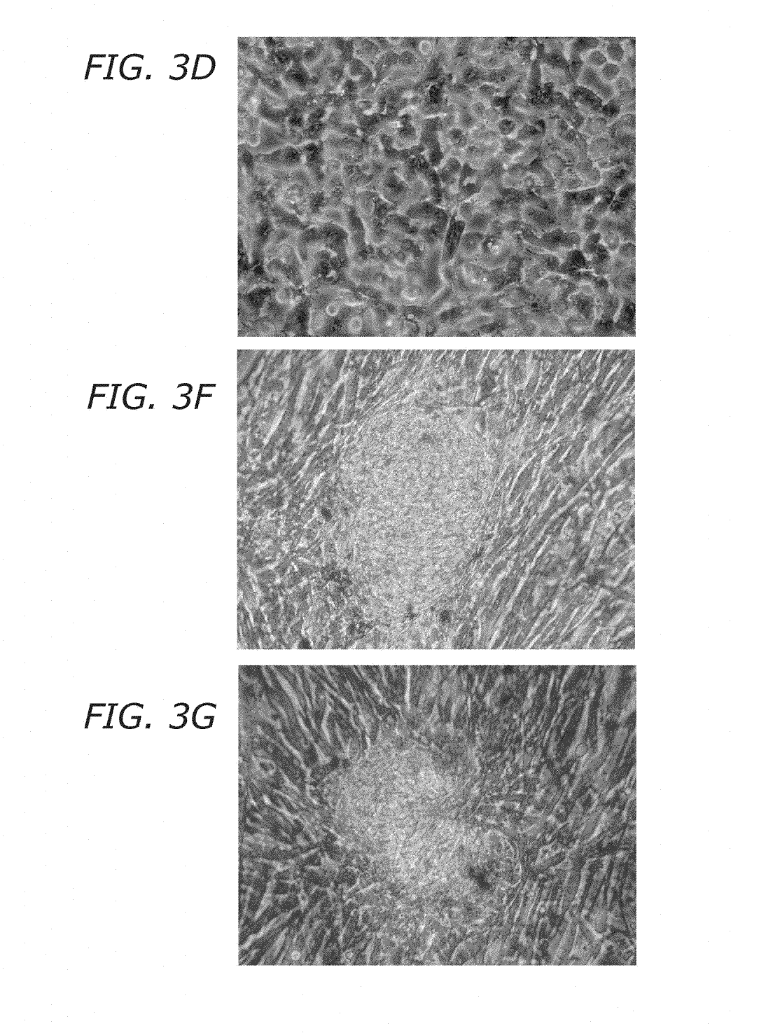

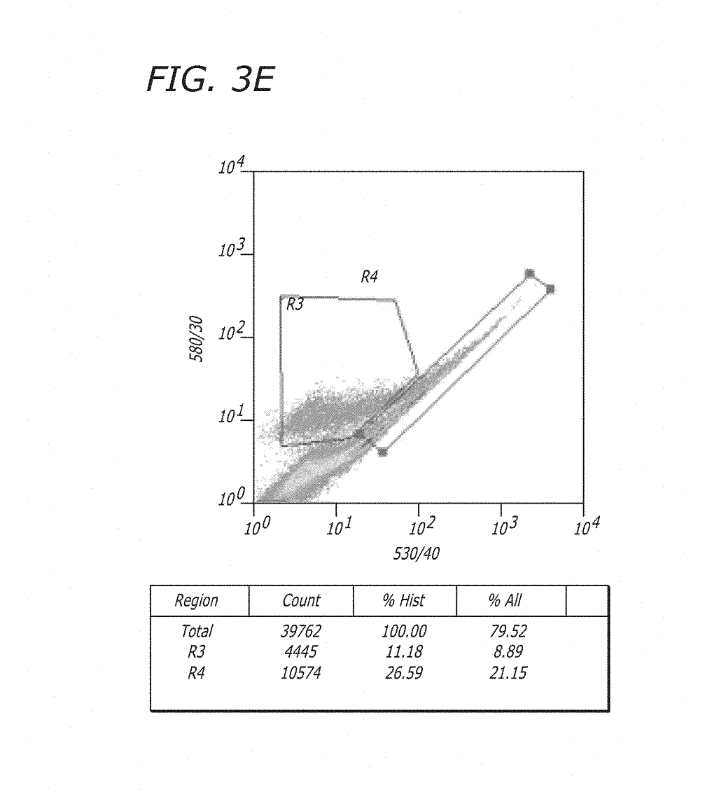

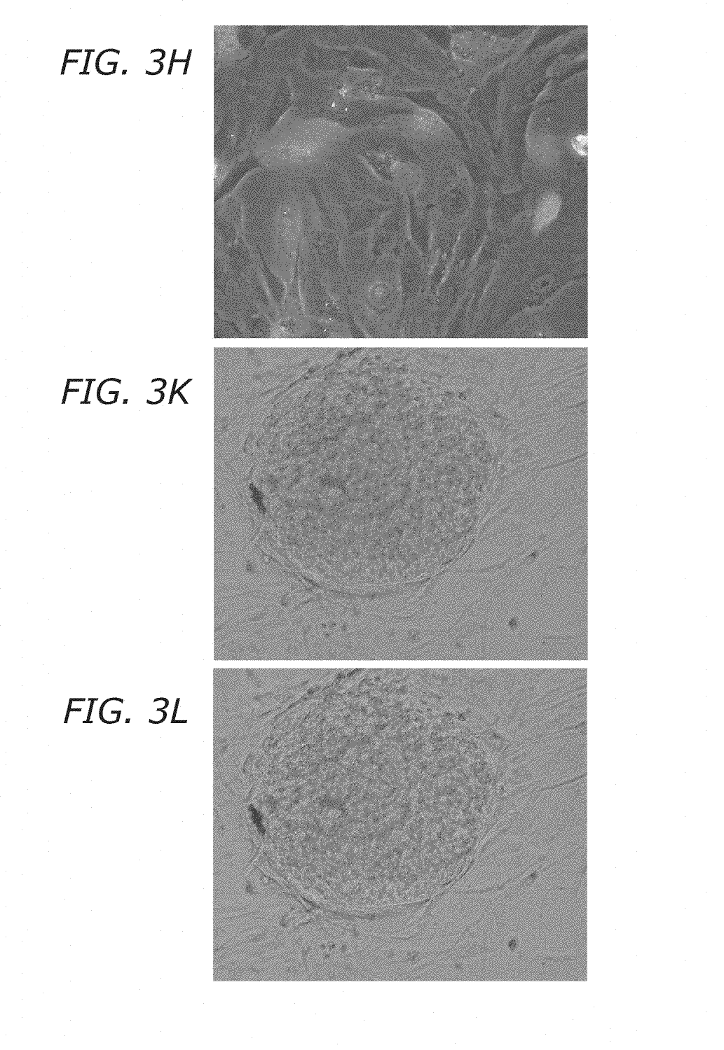

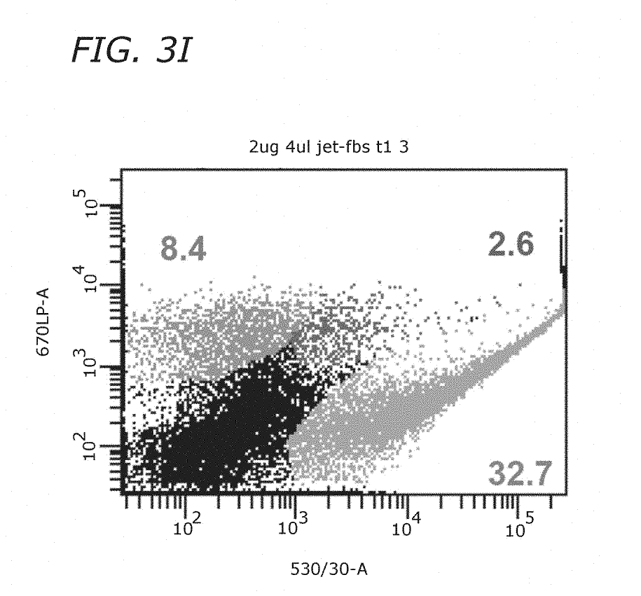

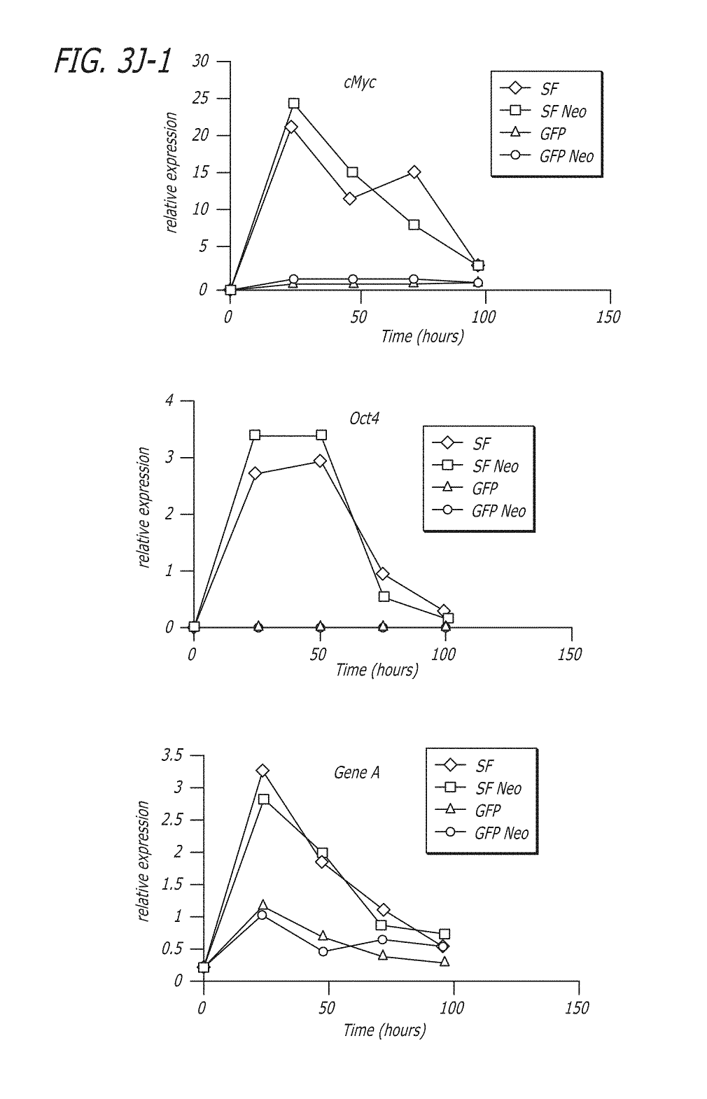

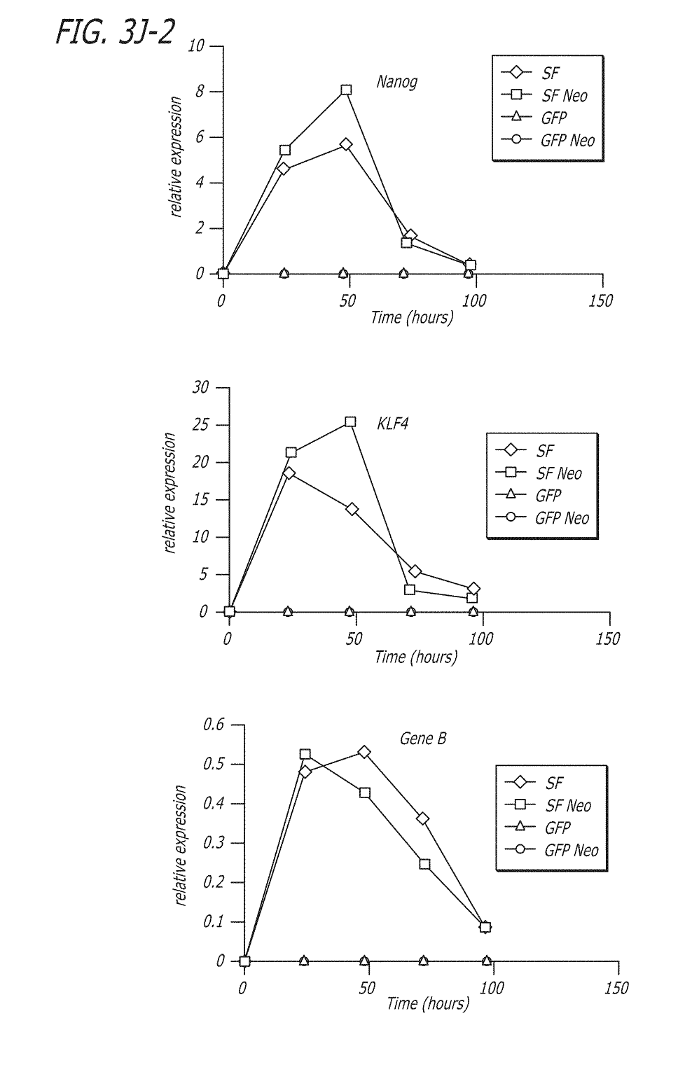

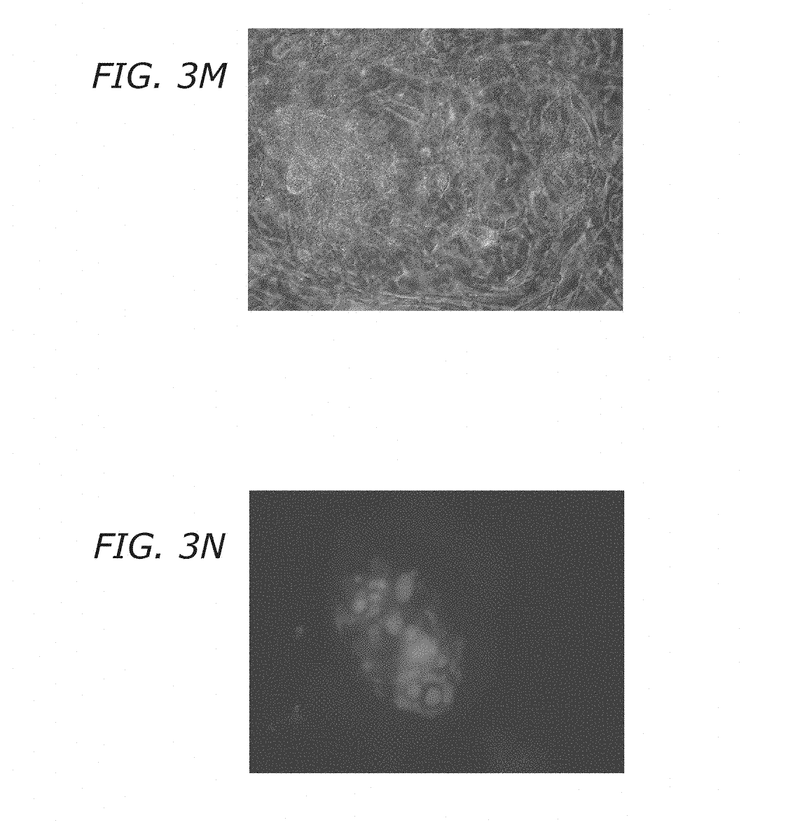

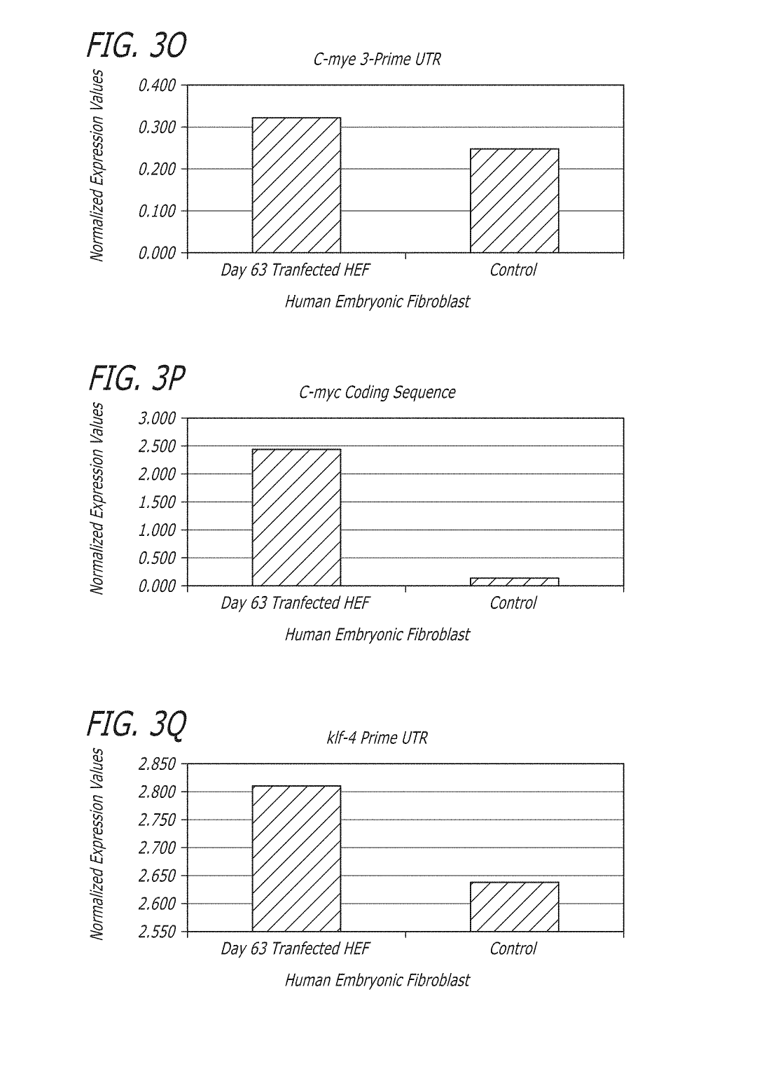

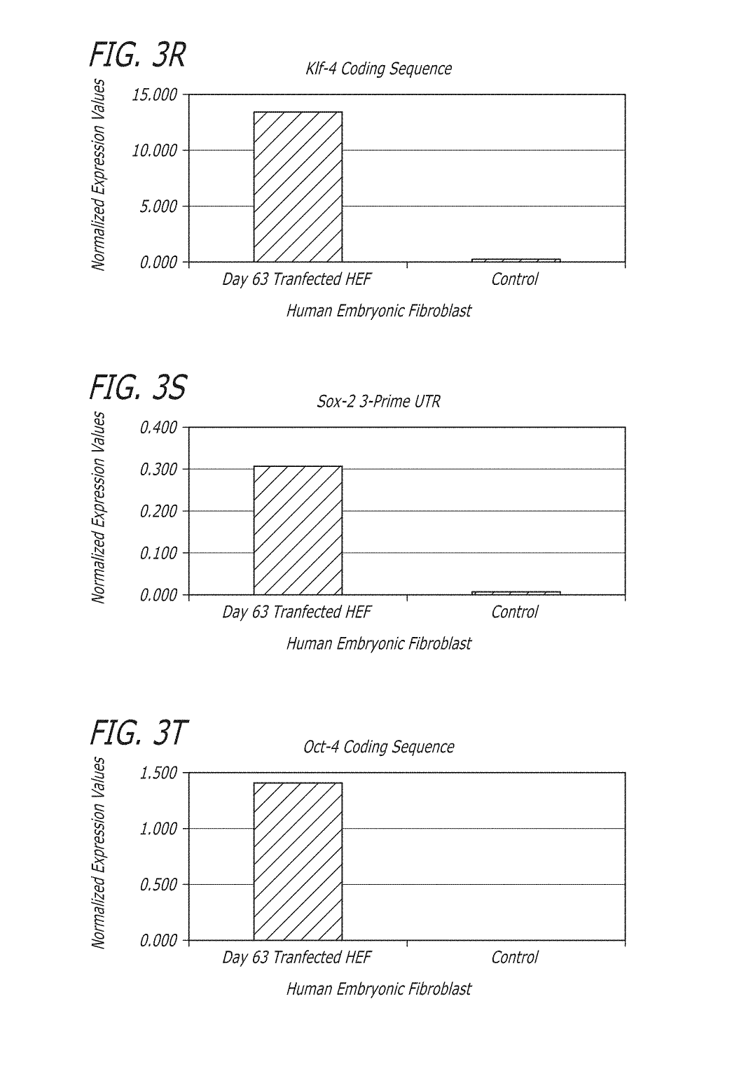

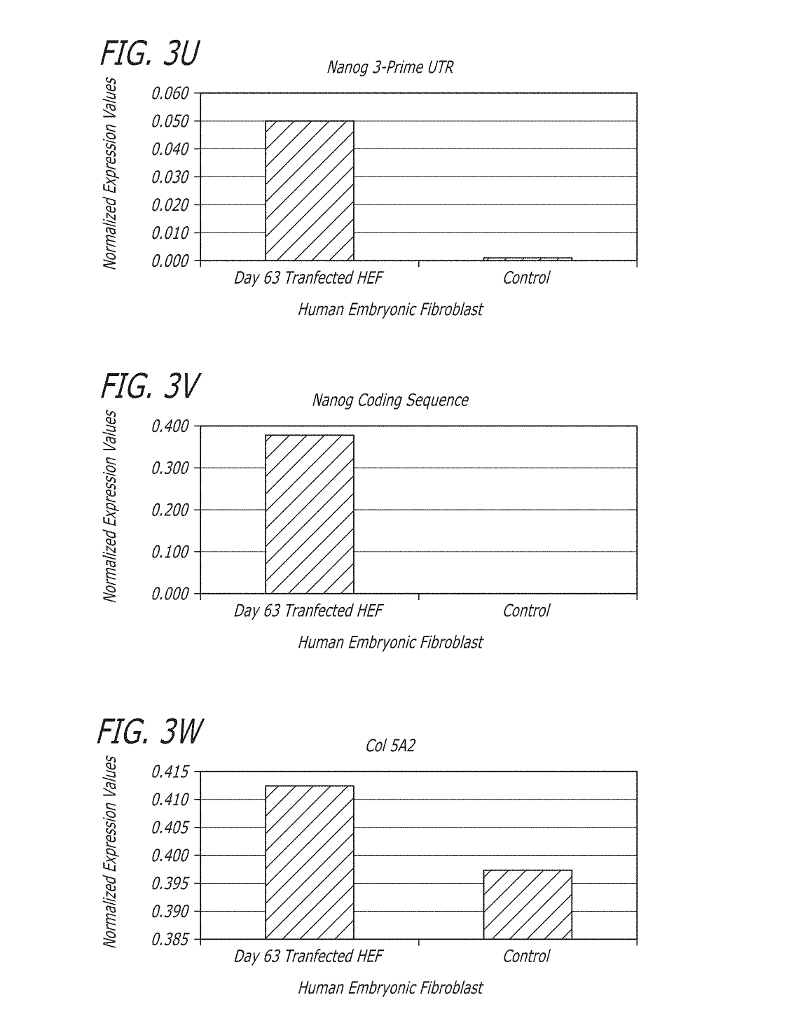

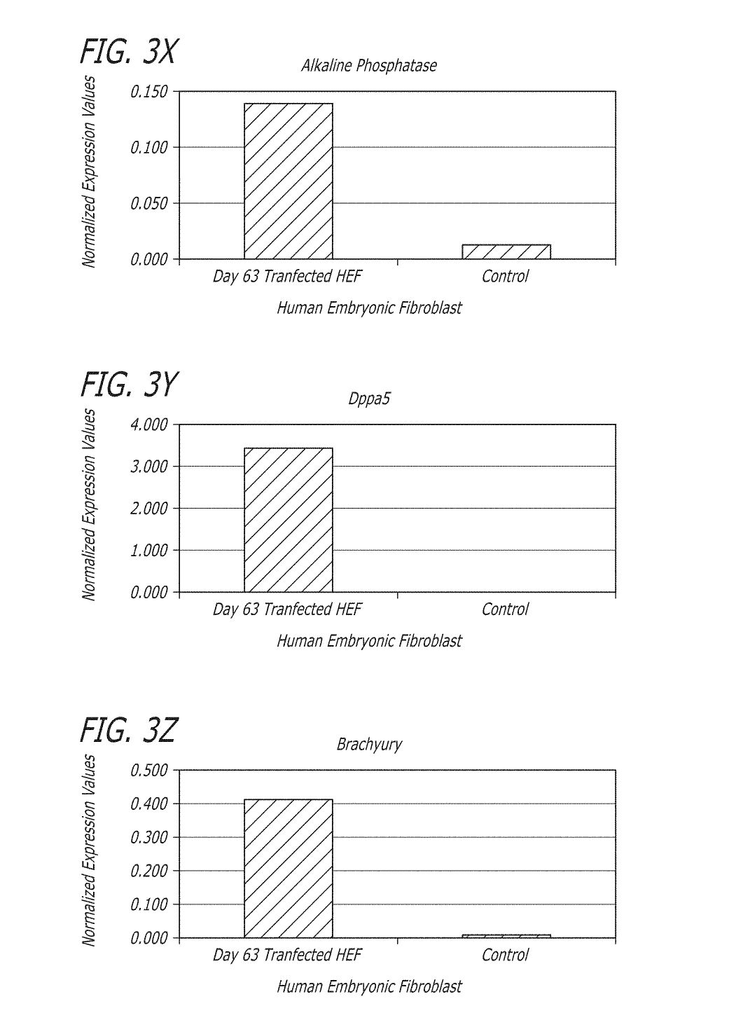

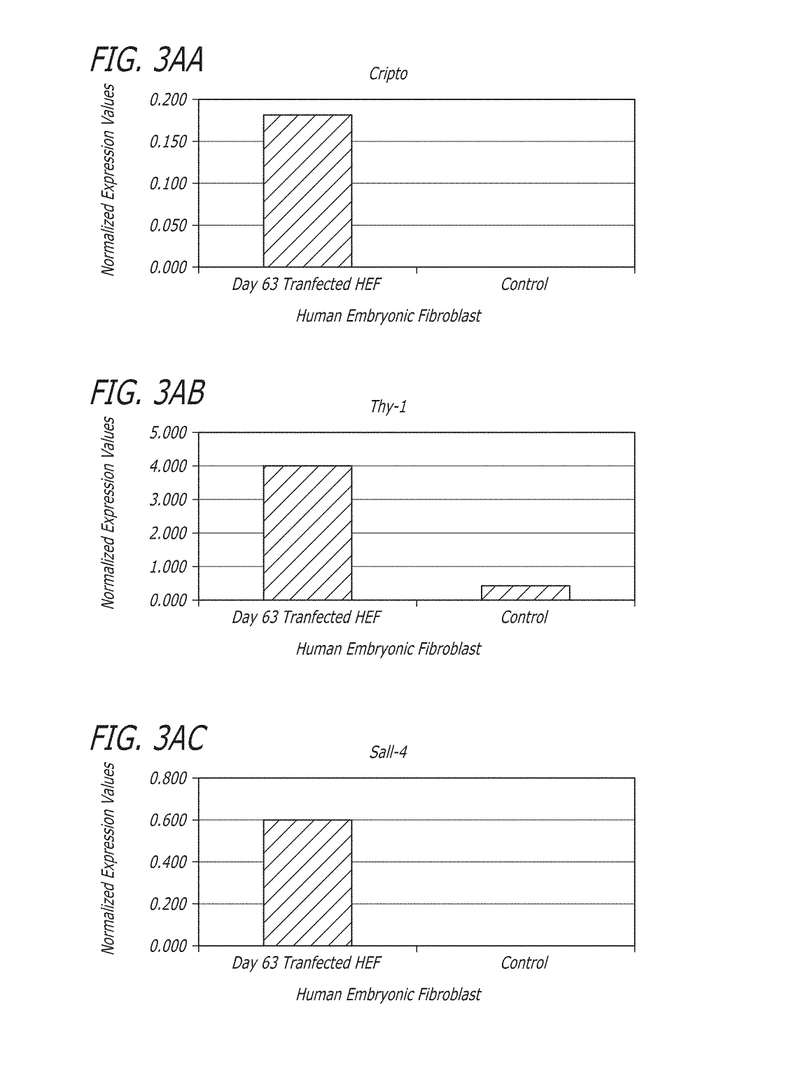

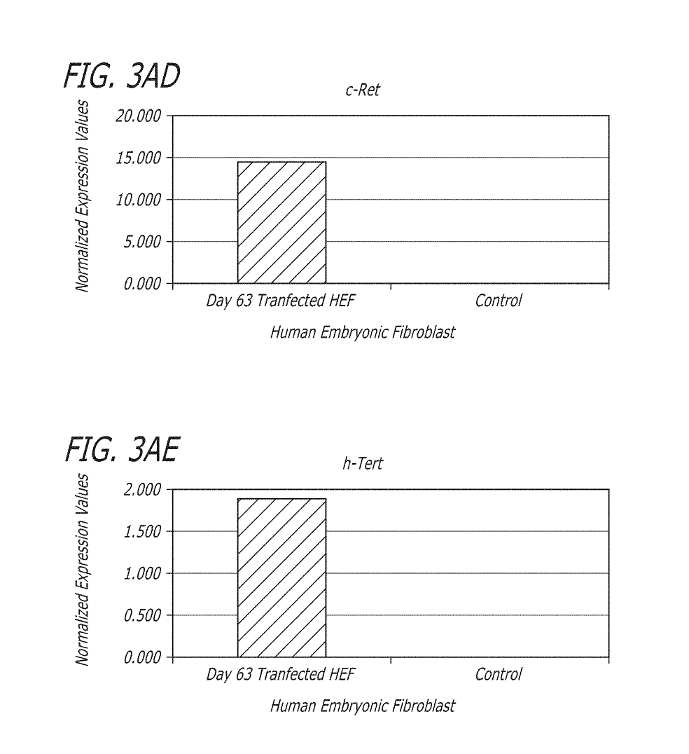

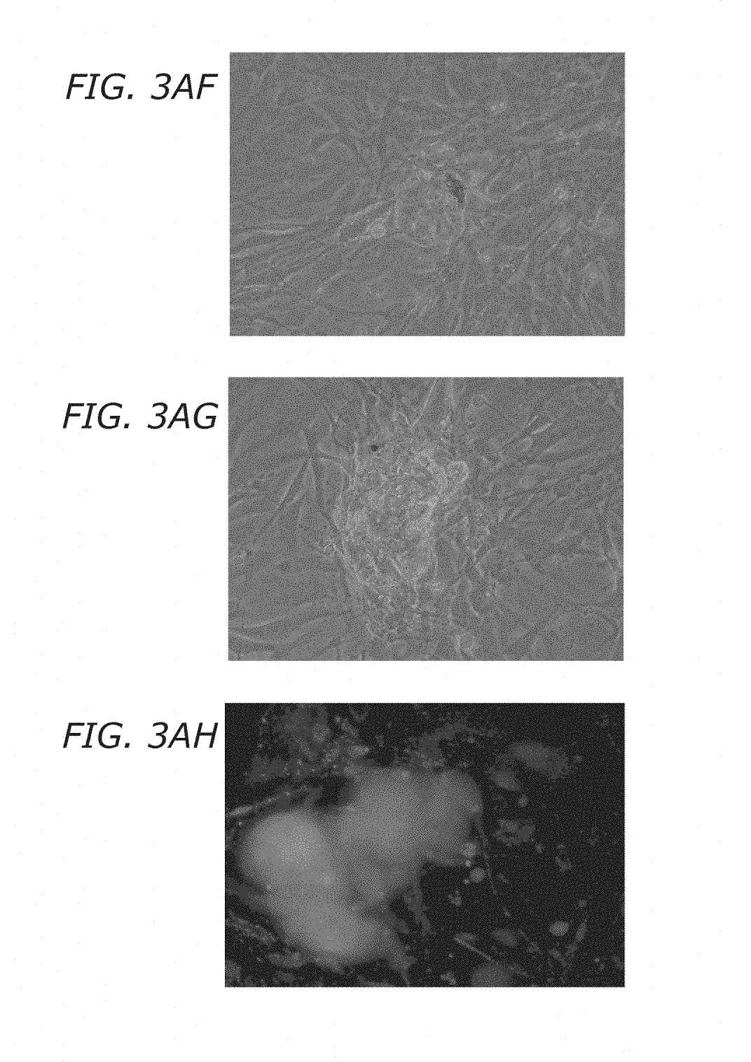

Within 3 days after transfecting the HFF cells with 5 TFactor DNA/SWNT and plating on mitomycin C-treated MEFS, the transfected HEF cells started to formed ES-cell like colonies (FIG. 3A).

A series of experiments were conducted to test the abilities of 5 TFactor DNAs attached to SWNTs to induce colony formation and stem cell growth in various different human cells including human epithelial keratinocytes (HEK), rentinal pigment epithelial cells (RPE), and human testicular fibroblast Nanog reporter cells (HT-42) as described in FIGS. 3B-3G. Colonies were maintained in hESC-CM on mitomycin treated MEFs.

FIG. 3B depicts colony formation from HEK cells treated with 5 TFactor DNA/SWNT at Day 6. In experiments with RPEs, FIG. 3C depicts colony formation from RPE cells treated with 5 TFactor DNA/SWNT at Day 6 and FIG. 3d depicts expression of SSEA-4, a pluripotent marker, by these cells at day 14. FIG. 3E depicts FACS analysis of the SSEA-4 positive population in RPE cells transfected with 5 TFactor DNA/SWNT. RPE cells were trypsinized into a single cell suspension and live-stained with FITC-coupled anti-SSEA-4 antibody in normal growth medium. Propidium iodide (PI) staining was used to gate out dead cells during FACS. FACS analysis demonstrated that approximately 21% of the live cells stained positive for SSEA-4 after treatment with 5 TFactor DNA/SWNT at Day 14.

FIG. 3F depicts colony formation of human testicular fibroblast Nanog reporter cells (HT-42) treated with 5 TFactor DNA/SWNT at Day 6 in hESC-CM on mitomycin treated MEFs (20× magnification). FIG. 3G depicts colony formation from HT-42 cells treated with 5 TFactor DNA/SWNT at Day 6, showing Nanog upregulation indicated by the red fluorescent protein (RFP) expression as a reporter (the Nanog promoter drives expression of RFP). Colonies are autofluorescing (20× magnification).

Remarkably, some cell types expressed pluripotent markers, such as SSEA-4 and upregulate Nanog, another pluripotent marker. These results show the potential of 5 TFactor DNA/SWNT to reprogram cells and lead to the formation of colonies.

To investigate the significance of PEI delivery particle, HEFs were treated with the 5 TFactor DNA and/or GFP DNA coated onto PEI particles instead of SWNT. To bind DNAs to PEI particles, the following methods were used: For each well, 1-2 μg of DNA (total) was diluted into 100 μl of 150 mM NaCl solution, vortexed gently and briefly centrifuged to collect all the solution at the bottom of the tube. For each well to be transfected, 2-4 μl of PEI particle solution was separately diluted into 100 μl of 150 mM NaCl, vortexed gently and briefly centrifuged. The 100 μl PEI particle solution and the 100 μl DNA solution all at once and the mixture was vortexed immediately and centrifuged briefly to bring drops to the bottom of the tube. The solutions were then incubated for 15 to 30 min at RT. Two hundred microliters of the PEI particle/DNA mixture was added drop-wise into the 2 ml of serum containing medium (HEF media) in each cell-containing well and the solutions mixed by gently swirling the plate. The plates were then incubated at 37° C. in 5% CO₂ in a humidified atmosphere for 4-24 hr. The PEI particle/DNA mixture was then washed from the cells, fresh hESC media added and the cultures returned to the incubator.

To test the transfection efficiency, HEF cells were transfected with GFP plasmid DNAs coated onto PEI particles. The GFP expression in HEF cells was assessed two days after transfection (FIG. 3H). To quantify transfection efficiency, expression levels of GFP after transfection was measured using FACS analysis. Three days after transfection, the cells were trypsinized into single cells and analyzed for GFP expression; propidium iodide staining was used to gate out dead cells during FACS. FACS data showed that the transfection rate of approximately 32% (FIG. 3I) with PEI was higher in comparison to SWNT-mediated transfection. To measure the levels of expression of each gene after transfection with 5 TFactor DNAs, the multiplex RT-PCT (XP-PCR) was performed at various time points from 0-94 hr (FIG. 3J). The expression levels of the 5 TFactors increased for 24 hr after infection, and then dropped after 72 hr (FIG. 3J). To maintain upregulation of the 5 TFactor DNAs, continuous multiple transfections with PEI particles were used every 2-4 days, for up to 4 rounds of transfections. At day 15, after the fourth and final round of transfection, transfected HEF cells were plated at 1×10⁵ cells on 10 cm mitomycin C-treated MEF plates in hESC medium. By day 23, the transfected HEF cells were observed forming ES-like colonies (FIG. 3K). These colonies were tested for various pluripotent markers (SSEA-4, TRA1-60 and alkaline phosphatase) to assess if reprogramming occurred. Colonies were live stained with TRIC coupled anti-SSEA-4 antibody in normal growth media (FIG. 3L), FITC-coupled anti-TRA1-60 antibody (FIG. 3M). Alkaline phosphatase activity was assed by fixing cells in 2% paraformaldehyde, washing 2× in PBS and staining with Vectastain ABC Alkaline phoshatase enzyme activity kit.

To assess if the observed colonies were a contamination of MEF cells that escapted mitomycin C treatment, a dual staining of alkaline phosphatase and FITC-coupled anti-human nuclei antibody was performed. The colonies showed positive staining human nuclei and for alkaline phosphatase activity (FIG. 3N). This demonstrated that transfected HEF cells were of human origin and expressed the pluripotent marker alkaline phosphatase. To further analyze the colonies, 63 day old HEF cell colonies were tested for gene expression by multiplex RT-PCR and multiple pluripotent genes were up-regulated including c-Myc (FIGS. 30 and 3P), Klf4 (FIGS. 3Q and 3R), Sox2 (FIG. 3S), Oct-4 (FIG. 3T), Nanog (FIGS. 3U and 3V), Col5A2 (FIG. 3W), alkaline phosphatase (FIG. 3X), Dppa5 (FIG. 3Y), Bachyury (FIG. 3Z), Cripto (FIG. 3AA), Thy-1 (FIG. 3AB), Sall4 (FIG. 3AC), cRet (FIG. 3AD) and hTERT (FIG. 3AE). These findings provide support for the assertions that HEF cells are responsive to reprogramming signals using PEI particles with 5 TFactor DNAs and that these transcription factors induce up-regulation of endogenous pluripotent genes resulting in expression of pluripotent markers by the cells. Thus in one embodiment, HEF cells, a cell type previously believed to be terminally differentiated, can be reprogrammed to become pluripotent stem cells by introduction into the cell of DNAs ecoding five different pluripotent stem cell transcription factors.

Furthermore, the ability of 5 TFactor DNA/PEI particles to reprogram other cells was tested by transfecting 5 TFactor DNA/PEI particles into additional cell types including rentinal pigment epithelial (RPE) cells and human testicular fibroblast Nanog reporter cells (HT-42) and colony formation and stem cell growth was seen (FIGS. 3AF-3AH). FIG. 3AF depicts colony formation of HT-42 cells treated with 5 TFactor DNA/PEI particles at Day 13 (20× magnification). FIG. 3AG depicts the colony formation of HT-42 cells treated with 5 TFactor DNA/PEI particles at Day 13, showing RFP expression from the Nanog promoter locus (20× magnification). FIG. 3AH depicts colony formation in the same cells at Day 56 wherein the colonies are positively stained for alkaline phosphatase and human nuclei after one round of transfection.

### Example 11

### Screening Methods for Identifying Conditions Effective for Reprogramming with Pluripotent Stem Cell Transcription Factor DNAs or Candidate DNAs, RNAs and Proteins

Disclosed above are highly efficient methods for delivery of DNAs, RNAs and proteins in a manner effective to induce rapid therapeutic reprogramming of somatic cells, e.g., 6 to 10 colonies per 20× microscopic field (Example 10). Because large numbers of colonies are observed within a relatively short period of time (3-7 days), screening assays are disclosed for proteins, RNAs and DNAs that in combination are effective to induce therapeutic reprogramming in somatic cells. The following assay is provided as one example:

1. Dermal punch biopsy samples are obtained from human subjects, minced and treated with collagenase and trypsin for 30 min/37° C. to release cells into suspension; tissue debris are removed by centrifugation at low speed and/or by unit gravity settling and the resultant supernatant cell suspension is collected by centrifugation, washed with D-MEM/10% FBS; and, established in tissue culture multiwell plates. After overnight incubation at 37° C. in 5% CO₂/95% air, the non-adherent cells are removed by decanting and the adherent cells returned to culture in D-MEM/10% FBS for 3 to 4 days;

2. Test proteins, RNAs and/or DNAs are conjugated to SWNT as described supra and added to the fibroblasts established in the multiwell plates, above. For example, cytoplasmic or nuclear extracts, and fractions thereof, from ESCs are conjugated to SWNT and introduced into test cells as described supra;

3. The test cells are cultured e.g. in D-MEM/10% FBS (5% CO₂/95% air) and monitored for development of colonies over the course of about 3 to about 7 days. The formation of colonies establishes that the mixture of proteins, RNAs and/or DNAs is a candidate mixture for inducing therapeutic reprogramming; and,

4. That the candidate mixture of proteins, RNAs and/or DNAs induces therapeutic reprogramming to produce pluripotent stem cells is established by (a) testing for the unlimited growth ability of the cells in the colonies by passaging the cells continuously and determining that their growth rate does not decrease with time and (b) by determining that the cells in the continuous cell cultures express pluripotent stem cell protein expression markers by immunochemistry and Western blotting, RT-PCR markers, and/or biological activities of pluripotent stem cells such as the ability of the cells to differentiate into cells of all three developmental lineages ectoderm, mesoderm and endoderm.

In one highly desirable modification of this protocol, at Step 2, the mixture of test proteins, RNAs and/or DNAs contains also a marker gene that is under the control of a promoter element of a pluripotent stem cell transcription factor, e.g., a promoter region of Oct-4, Sox-2, Nanog or Klf-4. Marker genes are well known in the art and include, but are not limited to, fluorescent proteins such as GFP (green fluorescent protein), RFP (red fluorescent protein), as well as, enzymes such as lac-Z and β-gal. In Step 2, the marker gene is introduced into the cell with the other test proteins, RNAs or DNAs; and, in Step 3, when pluripotent stem cell transcription factor expression is induced, marker gene expression is also induced resulting in “marked cells” red or green fluorescent cells, a technique useful for rapidly identifying colonies in Step 3.

### Example 12

### Reprogramming with Purified Transcription Factor Plasmid DNAs and Recombinant Proteins

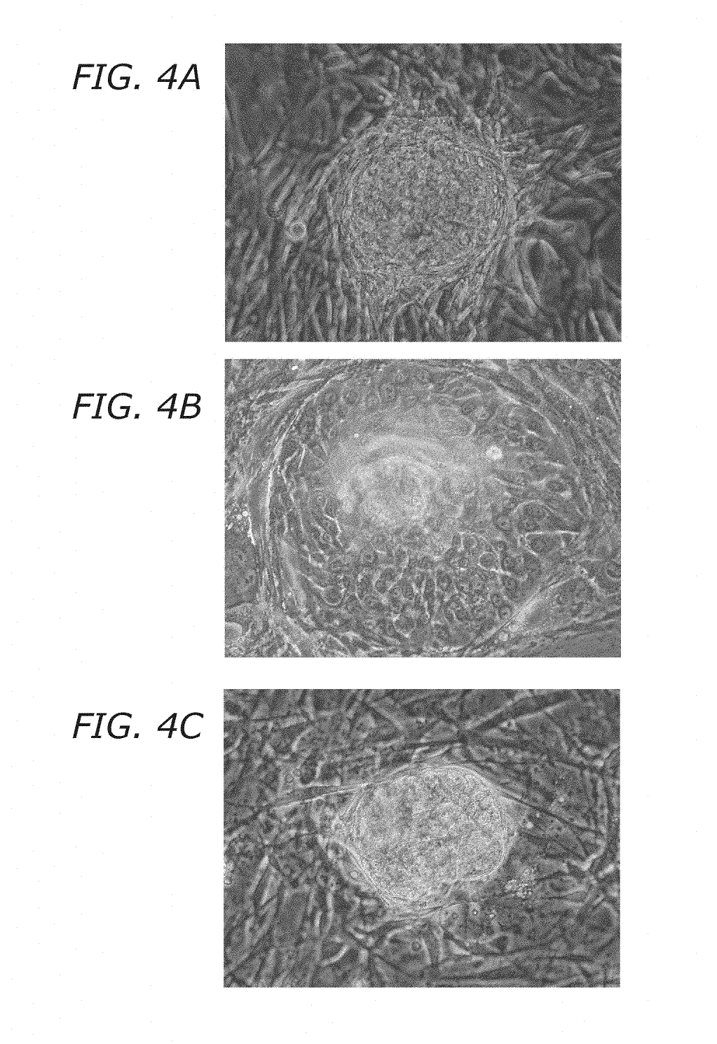

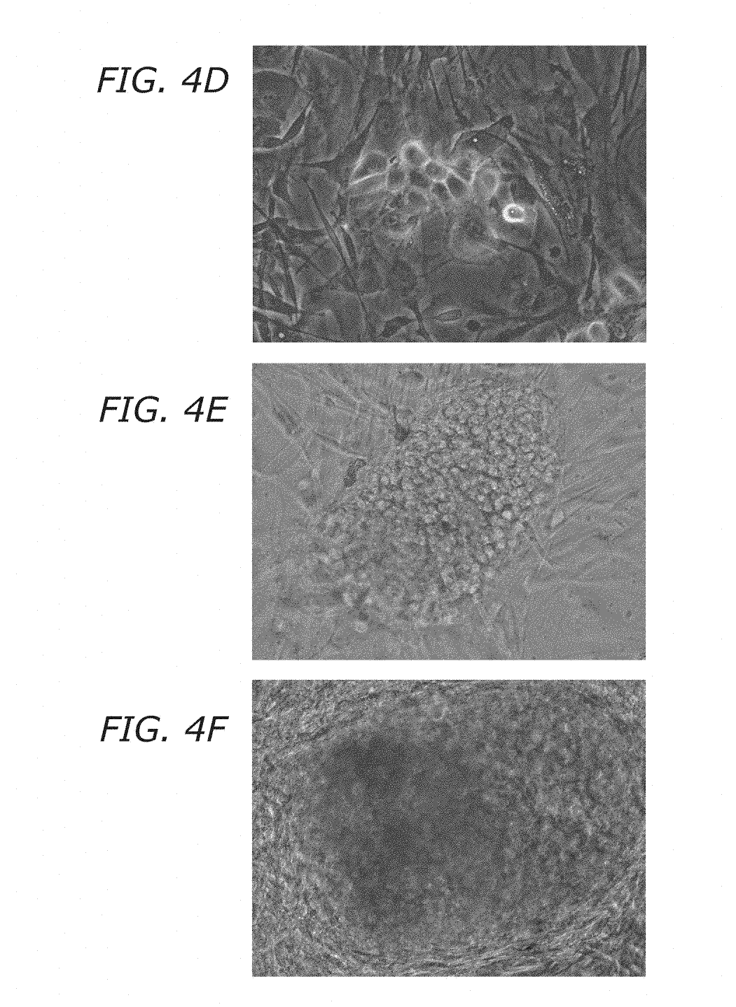

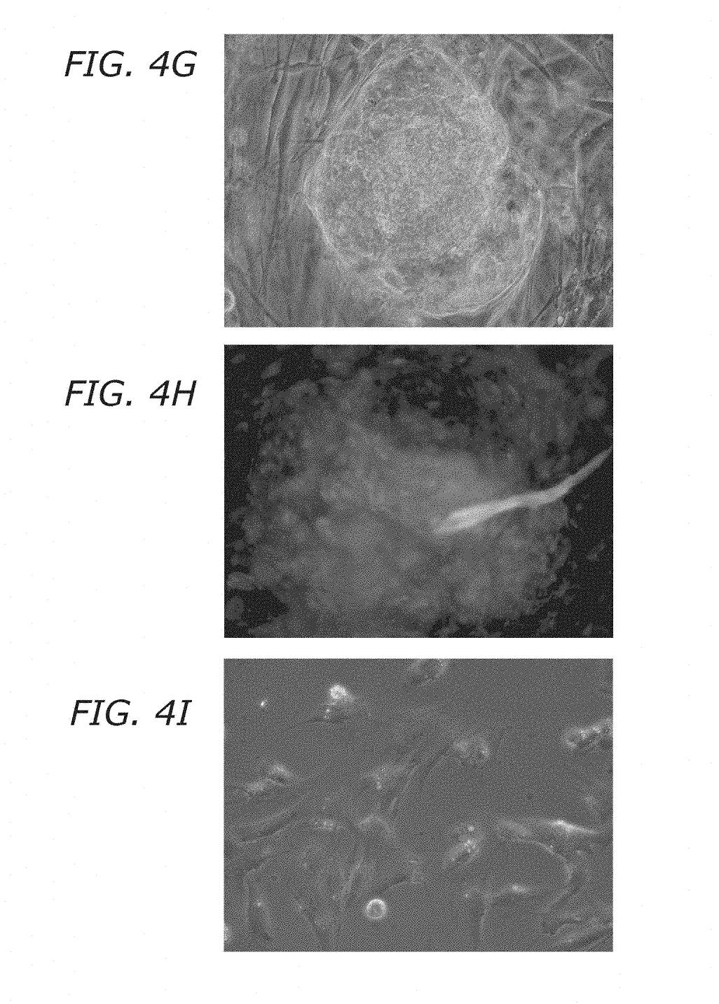

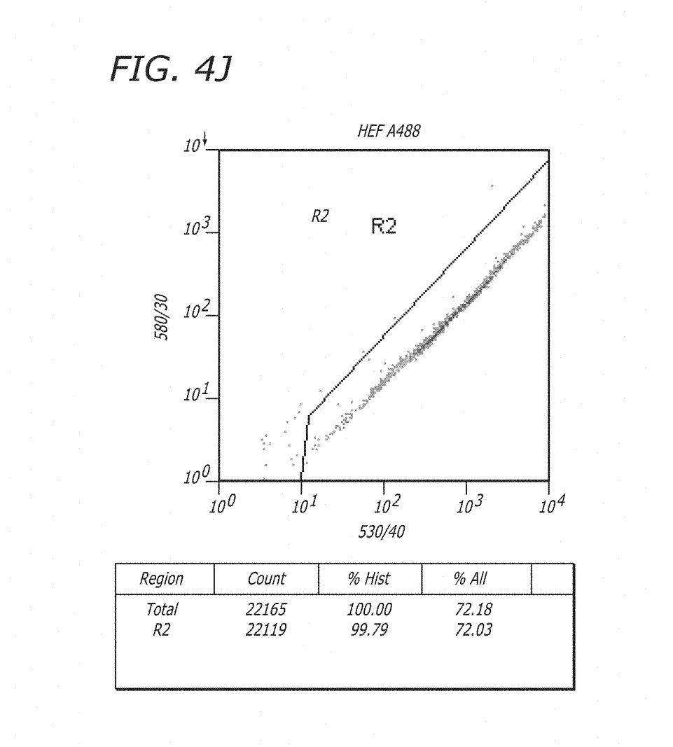

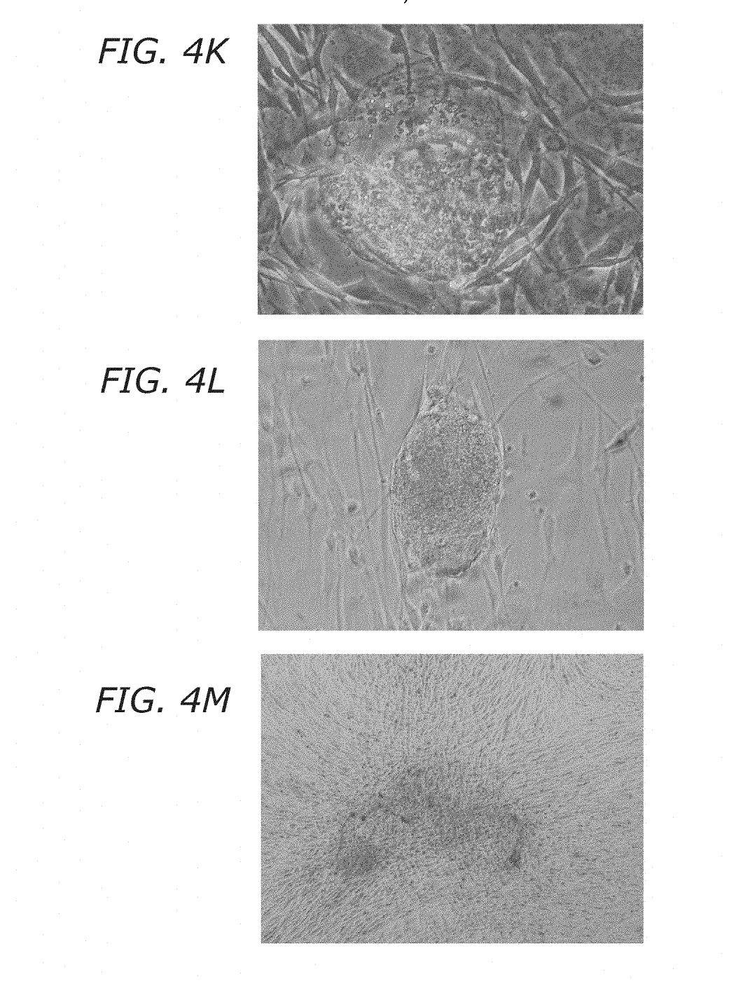

A series of experiments were conducted to test the abilities of 5 TFactor proteins to induce colony formation and stem cell growth in the different human cell types: HFF, HEK, RPE, and HT reporter cells, as described in FIGS. 4A-4M. Non-viral transfection of cell lines was accomplished by using 5 TFactor proteins attached to SWNTs or a cationic amphiphile molecule (PULSin™, Polyplus-Transfection, Inc., New York, N.Y.) that forms non-covalent complexes with proteins and antibodies. The PULSin™ complexes are internalized by anionic cell-adhesion receptors and are released into the cytoplasm, where they disassemble.

SWNT Binding to Proteins:

SWNT were prepared as in Example 10. Five micrograms of each individual 5 TFactor recombinant protein was added to a tube containing 100 μl of SWNT solution and the contents mixed well and incubated in the dark on ice for 2-3 hr with occasional agitation. After the incubation period, the contents of all five tubes were combined into one tube and triturated for 30-60 seconds with an insulin syringe. A 10 cm dish of 80% confluent HFF was washed twice with PBS and once with 150 uM DMEM+CQ (chloroquine), the media aspirated and 1-2 ml of 150 uM DMEM+CQ was added to just cover plate. Then, 500 μl of SWNT bound with the 5 TFactor protein was spread evenly across the plate followed by 500 μl of 300 uM DMEM+CQ. Optionally a further 1 ml of 150 uM DMEM+CQ can be added to fully cover plate if needed. The plates are incubated for 2-4 hrs in a 37° C./5% CO₂ incubator. The cells are then trypsinized, washed 2-3× with PBS and centrifuged at 400×g for 7 min. The cells were then counted and plated at 1×10⁵ per plate in a 10 cm mitomycin C-treated MEF feeders and cultured in hESC media in a 37° C./5% CO₂ incubator. Remarkably, within 6 days, HFF cells transfected with 5 TFactor protein/SWNT formed ES cell-like colonies. These colonies were observed growing in clumps within the spindle shaped fibroblast monolayer on mitomycin treated MEFs in hESC media (FIG. 4A).

Then a series of experiments were conducted to test the abilities of 5 TFactor proteins to induce colony formation and stem cell growth in the following different human cell types: HEK, RPE, and HT-42 as described in FIGS. 4B-4H. Some cell types were able to express pluripotent markers, such as SSEA-4, and up-regulate Nanog, another pluripotent marker after transfection. These results show the potential of 5 TFactor protein to reprogram cell and induce the formation of colonies.

FIG. 4B depicts colony formation from HEK cells treated once with 5 TFactor protein/SWNT at Day 6 in hESC-CM on mitomycin C-treated MEFs (20× magnification). FIG. 4C depicts colony formation from RPE cells treated once with 5 TFactor protein/SWNT at Day 13. FIG. 4D depicts colonies expressing SSEA-4, a pluripotent marker in RPE cells treated with once TFactor protein/SWNT at Day 64 in hESC-CM on mitomycin treated MEFs. The colony was live stained positive for FITC coupled anti-SSEA-4 antibody in normal growth media (20× magnification). FIG. 4E depicts colony formation of HT-42 cells treated once with 5 TFactor protein/SWNT at Day 6. FIG. 4F depicts colony formation from HT-42 cells treated once with 5 TFactor protein/SWNT at Day 18, showing Nanog up-regulation indicated by the expression of RFP as a reporter. Colonies were maintained in hESC-CM on mitomycin treated MEFs (20× magnification).

FIG. 4G depicts HT-42 colonies resulting from cells treated once with 5 TFactor protein/SWNT expressing SSEA-4 at Day 38. The colony was live stained positive for FITC-coupled anti-SSEA-4 antibody in normal growth media (20× magnification). FIG. 4H depicts colony formation from HT-42 cells treated with 5 TFactor protein/SWNT at Day 53, showing Nanog up-regulation indicated by the expression of RFP and also showing auto fluorescence of colonies (20× magnification).

In another method to transfect cells, PULSin™ particles were used to transfect cells with 5 TFactor proteins to induce reprogramming of those cells. These particles contain a cationic amphiphile molecule and deliver anionic proteins and antibodies to a large variety of eukaryotic cell lines including primary cells. The particles are most efficient when interacting with the protein by electrostatic and/or lipophilic interactions. Thus, anionic proteins (i.e. proteins with an isoelectric point <7) and antibodies are particularly well suited for delivery with these particles. However, delivery is not restricted to anions, as most proteins have a lipophilic core.

PULSin™ particle binding with proteins: Four micrograms of 5 TFactor proteins or Alexa 488 IgG antibody were diluted in 200 μl of 20 mM Hepes in a microcentrifuge tube, vortexed gently and centrifuged briefly. Sixteen microliters of PULSin™ particles were added, the mixture was again vortexed and centrifuged briefly. The protein/particle mixture was incubated for 15 min at RT. Cells were then washed once with 1×PBS or culture medium without serum and 3 ml of culture medium without serum was added. Then, 200 μl of PULSin™ particles/proteins were added to the cells and mixed by gently swirling the plate. The cells were incubated 37° C. in a 5% CO2 incubator for 4 hr, the medium containing the particle/protein complex was removed and replaced with fresh hESC media. The cells were analyzed immediately or after a day in culture.

To test the possibility that the proteins can be delivered into the cells using PULSin™ particles, Alexa 488 antibodies were bound to PULSin™ particles and transfected into HEF cells. Then, Alexa 488 fluorescence was determined in the transfected HEF cells immediately after transfection (FIG. 4I). To quantify transfection efficiency, fluorescence levels of Alexa 488 IgG antibody was measured using FACS analysis (FIG. 4J). HEF cells were trypsinized into single cells one day after transfection and live analyzed for Alexa 488 fluorescence. FACS data showed that the transfection rate of approximately 72% was slightly higher than with Alexa 488 bound to SWNT (see Example 10).

Next, HEF cells were transfected multiple times with 5 TFactor proteins/PULSin™ particles (for up to 5 continuous transfections, HEFs were transfected every 3 days over the period of 12 days). Within 29 days after the first transfection, ES-cell like colonies were observed growing in clumps within the spindle shaped fibroblast monolayer (14 days after the last transfection and culture on mitomycin C-treated MEFs in hESC medium, FIG. 4K). These colonies were further allowed to grow in hESC media and the growing colony was mechanically passed on day 49 onto new mitomycin C-treated MEFs and the colonies stained positive for the pluripotent marker SSEA-4 on Day 55 (FIG. 4LC). The growing colonies were live stained with TRIC coupled anti-SSEA-4 antibody in normal growth media demonstrating that the HEF colonies express cell surface pluripotent markers.

Another series of experiments were conducted to test the abilities of 5 TFactor protein/PULSin™ particles to induce colony formation and stem cell growth in HT-42 cells. HT-42 cells were able to form ES cell like colonies after 6 days cultured on 0.1% gelatin coated dishes in hESC media (FIG. 4M).

### Example 13

### Purified Recombinant Cell Permeable Transcription Factor Proteins for Non-Viral Induction of Cell Reprogramming

Cell penetrable peptides (CPP) have been described as promoting entry of proteins into cells via endocytic and non-endocytic pathways. Differences in delivery have been noted depending on the nature of the cargo protein, the choice of CPP and the cell type. When delivery is into the cellular endosome compartment a large portion of the protein cargo may be degraded by proteases; and, when delivery is non-endocytotic the protein may not be biologically active because it does not reach the necessary target.

To investigate whether CPP could deliver pluripotent stem cell reprogramming factors into adult human somatic and/or germ cells in a biologically active form, seven different CPP were initially chosen for construction of each of eight different recombinant reprogramming factor (RF) proteins: (1) VP22 from adenovirus; (2) Kaposi FGF signal sequence (kFGF); (3) protein transduction domain-4 (PTD4); (4) Penetratin; (5) M918; (6) TAT; and (7) Transportan-10. The reprogramming factors are Oct-4, Nanog, Sox2, c-Myc, Klf4, Lin28, Tert, Large T antigen. Recombinant proteins are engineered using commercially available expression vectors and established methods for production and purification in bacteria and/or yeast and/or mammalian cells. Briefly, the cDNAs coding for Oct-4, Nanog, Sox2, c-Myc, Klf4, Lin28, Tert, Large T antigen, are sub-cloned into various expression vectors, forming a multitude of RF-CPP constructs. Depending on the vector used, appropriate host cells are then transformed with each of the engineered RF-CPP expression vectors. Host cell clones that harbor the correct constructs are then induced to produce the RF-CPP recombinant protein (for example, IPTG is used to induce proteins in DE3 BL21 bacterial cells that are transformed with a bacterial expression vector). The resultant recombinant proteins are then extracted and purified from the host cells using routine methods described in the commercially provided instructions. The recombinant purified RF-CPP is then added directly to cell culture medium where they can now be transduced into the target cells. In the efforts to visually track the CPP mediated transduction, two proteins, green fluorescent protein (GFP) and red fluorescent protein (RFP), will also be fused to the various CPP.

Nucleotide sequences encoding the following CPPs are used in construction of the expression vectors:

VP22:
(SEQ ID NO: 1)
GGATCCCCACCAACGGCGCCAACCCGATCCAAGACACCCGCGCAGGGGCT
GGCCAGAAAGCTGCACTTTAGCACCGCCCCCCCAAACCCCGACGCGCCAT
GGACCCCCCGGGTGGCCGGCTTTAACAAGCGCGTCTTCTGCGCCGCGGTC
GGGCGCCTGGCGGCCATGCATGCCCGGATGGCGGCTGTCCAGCTCTGGGA
CATGTCGCGTCCGCGCACAGACGAAGACCTCAACGAACTCCTTGGCATCA
CCACCATCCGCGTGACGGTCTGCGAGGGCAAAAACCTGCTTCAGCGCGCC
AACGAGTTGGTGAATCCAGACGTGGTGCAGGACGTCGACGCGGCCACGGC
GACTCGAGGGCGTTCTGCGGCGTCGCGCCCCACCGAGCGACCTCGAGCCC
CAGCCCGCTCCGCTTCTCGCCCCAGACGGCCCGTCGAGCCACCACCACCA
GAATT
kFGF:
(SEQ ID NO: 2)
GCAGGATCCGGAGGAGCAGCAGTTGCACTACTACCAGCAGTTCTACTAGC
ACTACTAGCACCAGGAGGAGAATTCGCA
PTD4:
(SEQ ID NO: 3)
GCAGGATCCGGAGGATATGCACGTGCAGCAGCACGTCAAGCACGTGCAGG
AGGAGAATTCGCA
PENETRATIN:
(SEQ ID NO: 4)
CGCCAGATTAAAATTTGGTTTCAGGGACGCCGCATGAAATGGAAAAAA
TAT
(SEQ ID NO: 5)
TACGGTCGTAAAAAACGTCGTCAGCGTCGTCGT
M918:
(SEQ ID NO: 6)
ATGGTGACCGTGCTGTTTCGCCGCCTGCGCATTCGCCGCGCGTGCGGCCC
GCCGCGCGTGCGCGTG
TRANSPORTAN-10:
(SEQ ID NO: 7)
GCGGGCTATCTGCTGGGCAAAATTGGACTGAAAGCGCTGGCGGCGCTGGC
GAAAAAAATTCTG

The resultant recombinant purified CPP-TF proteins were as listed in Table 1.

The following experiments demonstrate non-virally induced epigenetic reprogramming using reprogramming factor-cell penetrating peptide fusion protein transduction.

1. Visualizing and Determining Localization of RF-CPP:

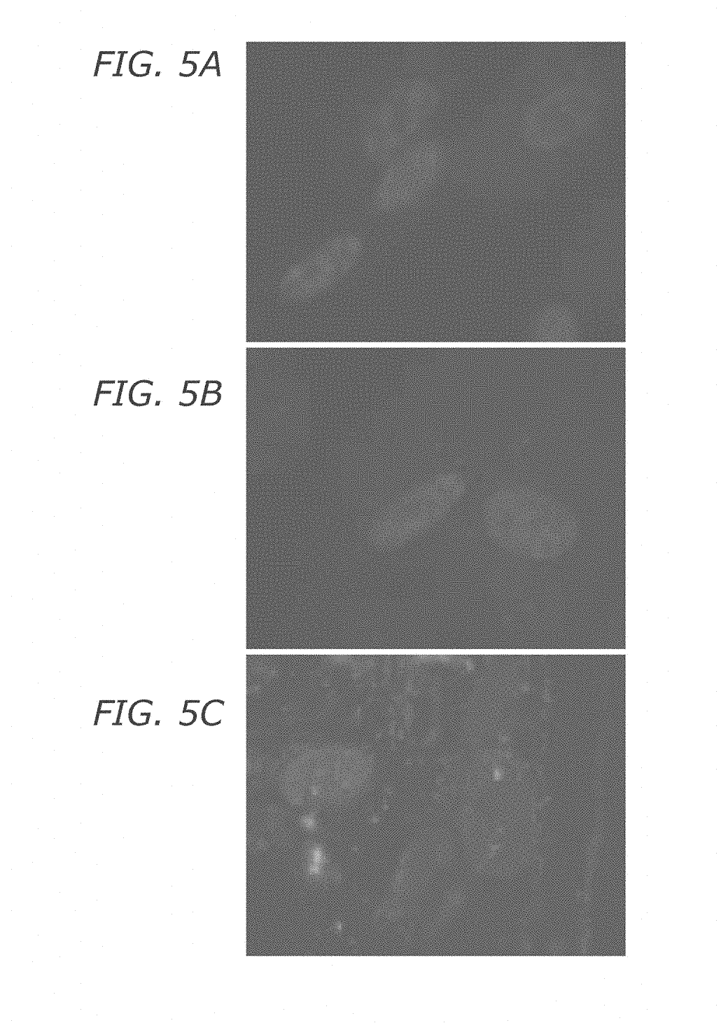

The RF-CPP fusion proteins outlined in Table 1 are expressed, harvested, and purified using one or a combination of bacterial, yeast, or mammalian expression hosts. Initially, to determine localization of RF-CPP transduction, Oct-4-Penetratin was used to transduce HEFs. HEFs were exposed (or not) to Oct4-Penetratin for 1 hr, then fixed in 3.8% PFA for 10 min, permeablized with 0.1% Triton for 2 min, blocked for 1 hr with 5% BSA in PBS and subjected to indirect immunofluorescence with an antibody targeting human Oct-4 (produced in rabbit) and a FITC conjugated-anti rabbit antibody. FIG. 5A depicts HEFs cells being transduced with Oct-4-Penetratin. It enters the cell and has a non-specific punctate localization. As shown in FIG. 5B, the Oct-4-Penetratin appears to be penetrating the membrane with a uniform localization. The nuclei are labeled with Hoechst 3342.

2. Verification of Nanog Promoter Activation:

The RF-CPP fusion proteins outlined in Table 2 then used to test whether the Nanog promoter can be activated by ectopic introduction of RF-CPP. Adult human somatic and/or germ cells are transfected with a DNA construct containing RFP driven by the Nanog promoter. Approximately 90,000 cells are plated per well in a 12-well dish and transfected with 2000 ng Nanog Promoter-RFP DNA. Twenty-four hours post transfection cells are then washed 3 times with PBS to release cells from transfection. To test for Nanog promoter activation (measured by RFP expression) combinations of RF-CPP are directly added to the cell culture medium, where the final concentration of each RF-CPP is 4 μM. Cells are monitored for RFP expression 6, 12, 24, and 48 hr post RF-CPP transduction. The RFP positive cells (Nanog activation) are then monitored by microscopy and FACS. This demonstrates that the RF-CPP are penetrating the cellular membrane and retaining their biologically active roles. RFP is expressed within a 24-72 hr time period post transduction of RF-CPP. Pilot experiments using the RF-CPP in the form of DNA constructs and subsequent co-transfections with the Nanog promoter shows Nanog promoter activation,

TABLE 1
Recombinant Cell Penetrable Pluripotent Stem Cell Transcription Factors

| GFP-VP22 | RFP-VP22 | Oct4-VP22 | Nanog- | Sox2-VP22 | cMyc-VP22 | Klf4-VP22 | Lin28-VP22 | Tert-VP22 | Large T- |
|---|---|---|---|---|---|---|---|---|---|
| | | | VP22 | | | | | | VP22 |
| GFP-kFGF | RFP-kFGF | Oct4-kFGF | Nanog- | Sox2-kFGF | cMyc-kFGF | Klf4 -kFGF | Lin28-kFGF | Ted-kFGF | Large T- |
| | | | kFGF | | | | | | kFGF |
| GFP-PTD4 | RFP-PTD4 | Oct4-PTD4 | Nanog- | Sox2-PTD4 | cMyc-PTD4 | Klf4-PTD4 | Lin28-PTD4 | Ted-PTD4 | Large T- |
| | | | PTD4 | | | | | | PTD4 |
| GFP- | RFP- | Oct4- | Nanog- | Sox2- | cMyc- | Klf4- | Lin28- | Ted- | Large T- |
| Penetratin | Penetratin | Penetratin | Penetratin | Penetratin | Penetratin | Penetratin | Penetratin | Penetratin | Penetratin |
| GFP-TAT | RFP-TAT | Oct4-TAT | Nanog-TAT | Sox2-TAT | cMyc-TAT | Klf4-TAT | Lin28-TAT | Ted-TAT | Large T- |
| | | | | | | | | | TAT |
| GFP-M918 | RFP-M918 | Oct4-M918 | Nanog- | Sox2-M918 | cMyc-M918 | Klf4-M918 | Lin28-M918 | Ted-M918 | Large T- |
| | | | M918 | | | | | | M918 |
| GFP- | RFP- | Oct4- | Nanog- | Sox2- | cMyc- | Klf4- | Lin28- | Ted- | Large T- |
| Transportan- | Transportan- | Transportan- | Transportan- | Transportan- | Transportan- | Transportan- | Transportan- | Transportan- | Transportan- |
| 10 | 10 | 10 | 10 | 10 | 10 | 10 | 10 | 10 | 10 |

observed by RFP expressing cells. FIG. 28 shows that the addition of RF-CPP in the form of DNA transfection increases the activity of the Nanog promoter in terms of RFP expression by 5.7 fold when compare to the Nanog promoter alone control.

3: Long-Term RF-CPP Transduction:

Adult human somatic and or germ cells are plated onto 12-well dishes at approximately 90,000 cells per well. Twenty-four hours later cells are subjected to ectopic transduction of RF-CPP. Combinations of RF-CPP are then directly added to the cell culture medium at a final concentration of 4 μM and cells are monitored for any changes in morphology. RF-CPP are refreshed on a daily basis, where medium is replaced with medium containing fresh RF-CPP. Seven days after the initial transduction the cells are trypsinized and replated onto mitomycin C inactivated MEFs at a concentration of 3500 cells per well in a 12-well dish. While in co-culture, RF-CPP are refreshed on a daily basis, with fresh medium containing RF-CPP. The cells are then monitored for the appearance of colony-like morphology. Once colonies are present, the cells are assayed for stem cells markers, including but not limited to, SSEA-4. SSEA-4 positive cells (assayed by live cell immunofluorescence) are mechanically picked and plated onto fresh MEFs and be further cultured to expand and propagate for cell line derivation. During derivation, cells are harvested for RNA and used for gene expression studies (GeXP) verifying endogenous transcription/reprogramming factor activation.

### Example 14

### Reprogramming for Cell Growth and Expansion of Cell Numbers without Reprogramming for Pluripotency

For certain clinical applications it is desirable to be able to increase cell numbers without reprogramming cells for pluripotency because such minimal modifications (a) reduce the risks of cancer and (b) maintains the epigenetic state of the cells making it easier to re-differentiate them back into the specialized cells of origin. This method is particularly useful where only small numbers of cells are available from patients. According to the instant methods the small numbers of cells are collected, reprogrammed for growth and expansion, then placed in media under conditions suitable and sufficient for cell growth and increase in cell numbers. When the numbers of cells are sufficient large, they are collected to formulate a therapeutic unit dose of cells, i.e., that dose needed to effect a positive clinical outcome in a subject in need.

Retinal pigment epithelial cells were reprogrammed for cell growth and expansion by lentiviral expression vector introduction of just two transcription factors, namely, Nanog and c-myc. The resultant reprogrammed cells changed their morphology to become small round cells, formed colonies and expanded rapidly over the course of 7-14 days. When passaged before they became confluent, the primary cell cultures formed continuously growing cell lines by day 20-30. The resultant continuous cell lines effectively down-regulated the lentiviral expression of Nanog and c-myc and, they did not express pluripotent stem cell RT-PCR markers, but, remarkably, these cells had endogenous up-regulated expression of Oct-4 (<33% the levels in ESC), Sox-2 (<10% the levels in ESC) and/or Nanog (<5% the levels in ESC).

Derivation of Intermediate Retinal Pigment Epithelial Cells and Human Embryonic Fibroblast iPS-Like Cells.

Induced pluripotent stem cells (iPS) have great potential to support regenerative and developmental research in conjunction with hESC. It has been thought that both iPS and hESC could ultimately lead to personalized cell replacement therapies. It has also been reported that ectopic expression of Oct-4 and c-Myc may be sufficient to induce hESC-like morphological changes in human fibroblasts without inducing pluripotency. Described herein is the derivation of a non-pluripotent cell line from RPE growing in hESC-like colonies by expressing only Nanog, c-Myc and KLF4. Additionally, the expression profile of these cells was compared with an iPS-like cell line derived from human embryonic fibroblasts. Furthermore, re-infection of the intermediate RPE cell line with the missing transcription factors is not sufficient to induce iPS cells.

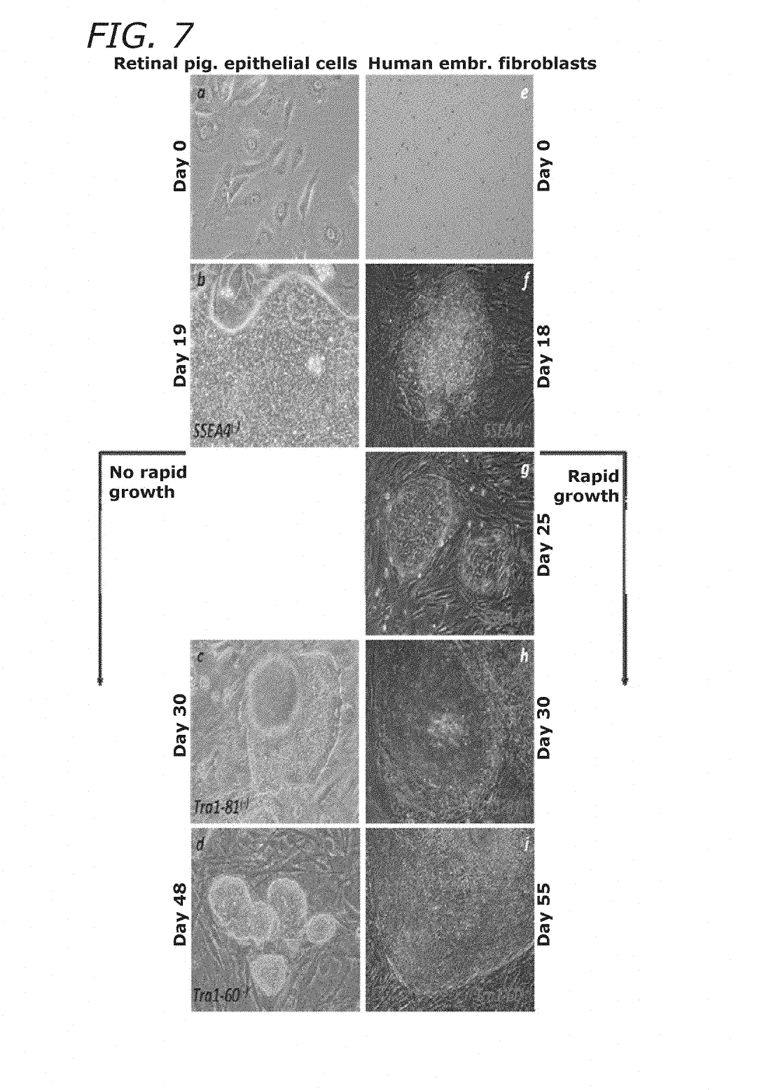

After lentiviral transduction of 5 transcription factors (Oct4, Sox2, Klf4, c-myc, Nanog) into RPE and HEF cells, colonies resembling embryonic stem cell colonies were detected after 18 days (FIG. 7). For RPE, 38 colonies were picked, while only four were detected and picked for HEF. Twenty of the RPE colonies and three of the HEF colonies were successfully expanded. While the RPE colonies could be treated with trypsin immediately at an early stage in expansion, HEF colonies had to be picked manually. During expansion, 10% of the RPE colonies and 80% of the HEF colonies developed a fibroblastic morphology. Taken together, the results suggest a more sustained and robust change in RPE as compared to HEF. Cell surface marker analysis of the colonies revealed that the RPE colonies did not stain for any embryonic stem cell marker, while the HEF colonies stained clearly for SSEA4, TRA1-81 and TRAAA1-60 (FIG. 7).

FIG. 7 depicts RPE cells grown in normal media before virus infection and at days 18, 30 and 48 and HEF cells before infection and at days 18, 25, 30 and 55 post-infection with lentivirus-containing Oct4, Sox2, KLF4, cMyc and Nanog. Cells were grown in culture medium for 6 days on a normal culture dish and subsequently seeded onto mitomycin C-treated MEF feeder cells at a density of 5×10⁴ cells. Colonies emerged after 18 days with a frequency of approximately 1/500 (RPE) and 1/10,000 (HEF). RPE colonies did not stain for SSEA-4 and could be picked and passaged onto new feeder cells. The resulting RPE colonies maintained their mophology, grew slowly and did not stain for TRA1-81. RPE colonies could trypsinized and passaged onto new feeder cells without losing their morphology and did not stain for TRA1-60. HEF colonies staining for SSEA-4 were manually picked and passaged onto fresh feeder cells and the colonies grew rapidly and maintained their expression of SSEA-4. The resulting HEF colonies also stained positive for TRA1-81 and TRA1-60. The colonies maintained their morphology at a rate of 20%. Differentiated cells were observed at the shape and size of fibroblasts.

Retinal Pigment Epithelial (RPE) Cells:

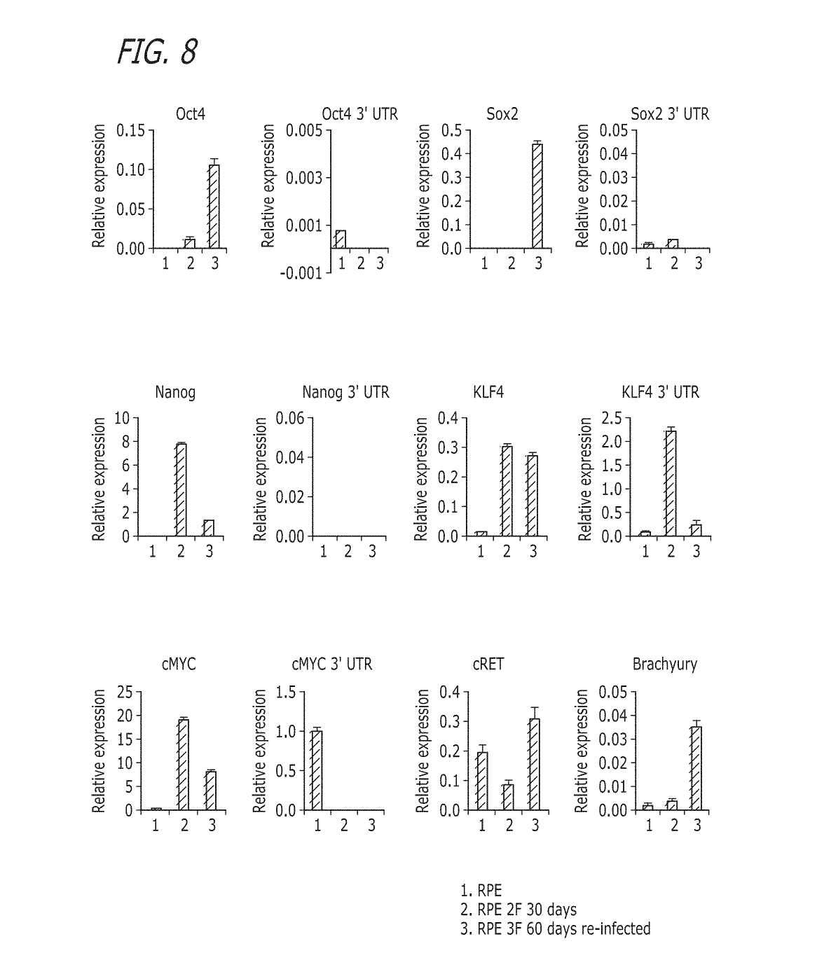

Gene expression analysis showed that control (GFP-transduced) RPE cells had low endogenous c-Myc and intermediate levels of KLF4 expression, but no expression of any other pluripotent markers (FIG. 8). After transduction and clonal expansion, one clone developed into a cell line, RPE clone-6, and expressed KLF4, c-Myc and Nanog, but lacked expression of Oct-4 and Sox2 cDNA, suggesting that just two transcription factors, cMyc and Nanog, were transduced into these cells. For RPE colonies, endogenous KLF4 expression rose after 5 factor infection as did total (endogenous+exogenous) KLF4 cDNA expression.

FIG. 18 depicts gene expression panel of retinal pigment epithelial cells grown in normal media before virus infection (Bar 1); RPE cells grown on mitomycin C treated mouse embryonic fibroblast feeder cells in hESC media at day 30 post infection with lentivirus containing Oct-4, Sox2, KLF4, c-Myc and Nanog virus (Bar 2) and RPE cells grown on mitomycin C-treated MEF feeder cells after two more rounds of subsequent virus infection with a combination of Oc-t4, KLF4 and Sox2 lentivirus (Bar 3).

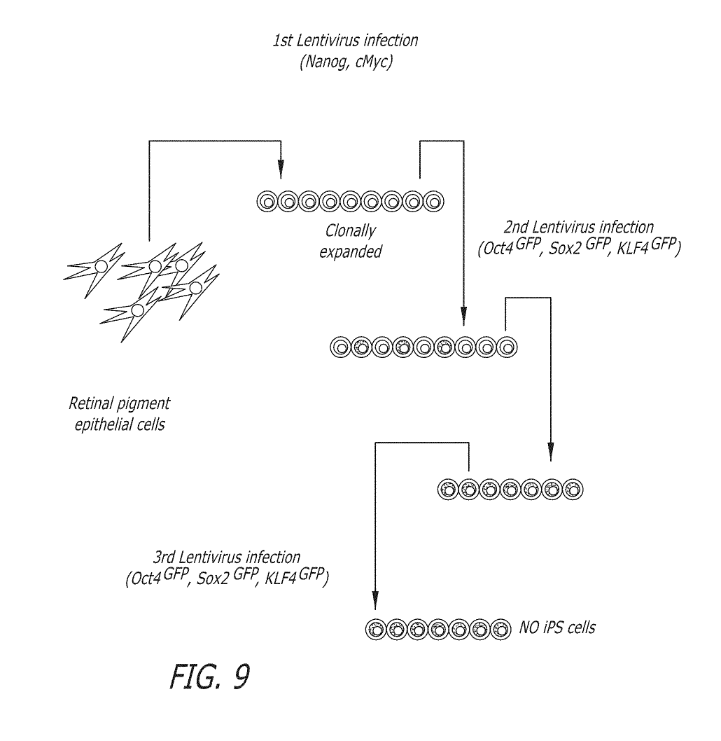

It was then determined whether the missing pluripotent transcription factors could be transduced a later time point. For these experiments, RPE colonies were transduced with lentiviruses having a bicistronic construct containing either KLF4, Oct-4 or Sox2 in combination with GFP (FIG. 9). Using this approach it was possible to infect the RPE colonies, sorting for GFP positive cells (ensuring that only infected cells were plated) and then infect again, thereby increasing the probability that all three transcription factors enter the cell. Gene expression analysis (FIG. 8) showed that re-infection lead to the uptake of Oct-4, Sox2 and KLF4 cDNA. However, there was no change in SSEA4 staining or endogenous pluripotent marker expression, showing that sequential infection under these conditions did not lead to an iPS state in these RPE cells in the observed time frame of 30 days.

Human Embryonic Fibroblasts (HEF):

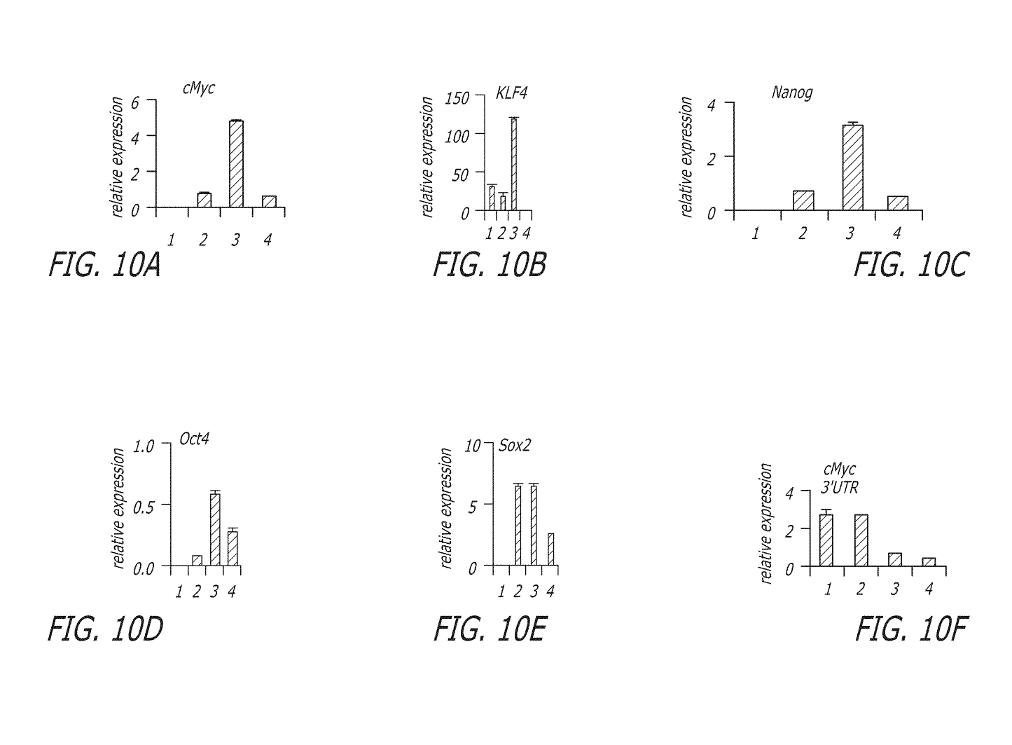

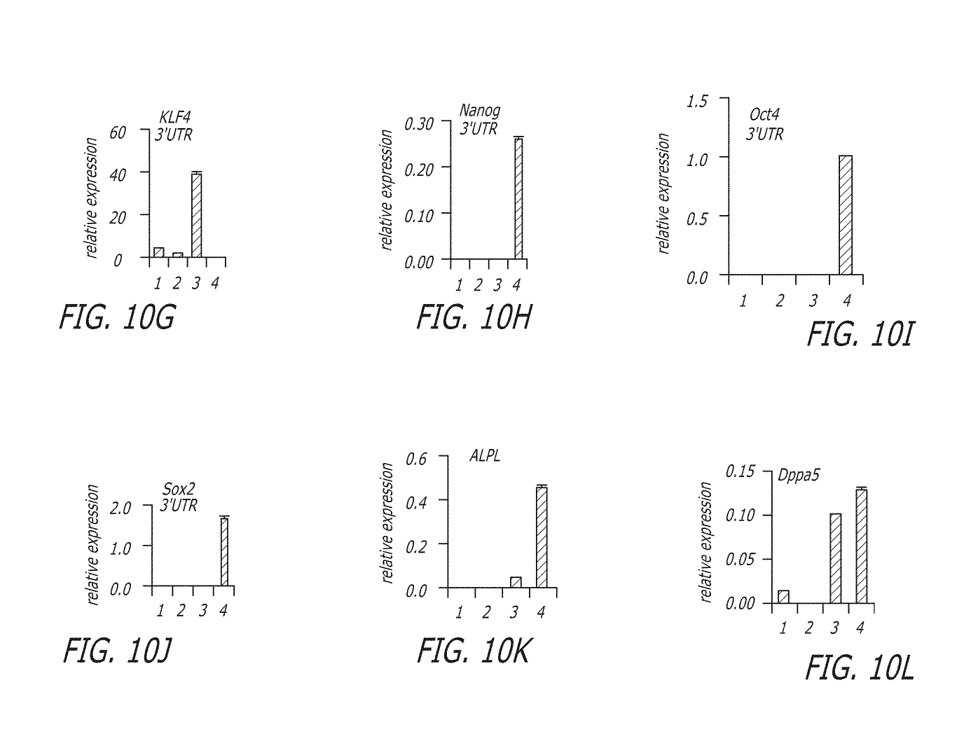

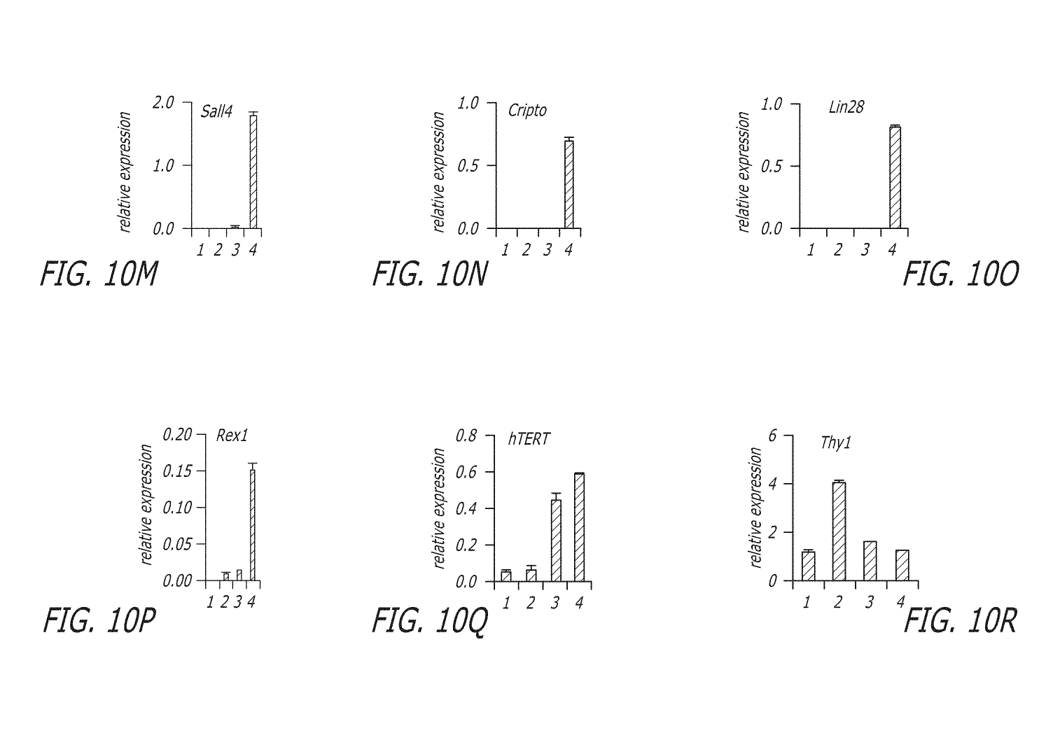

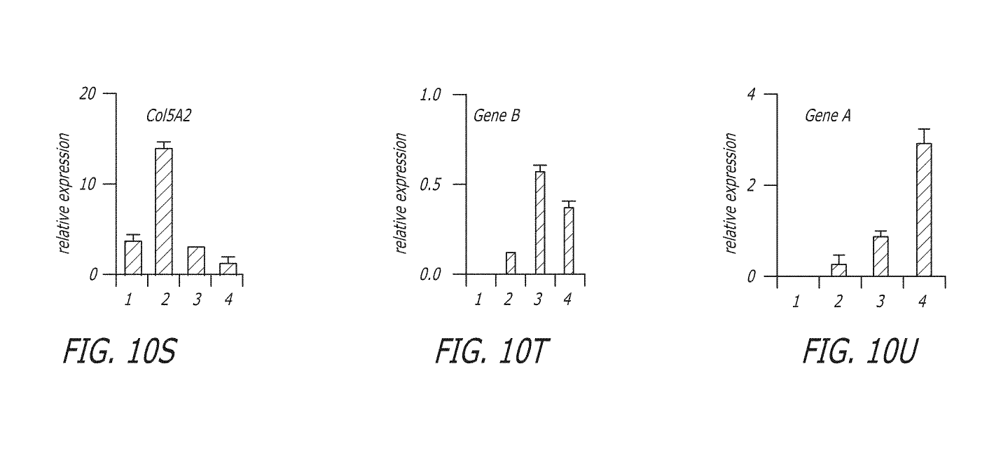

Gene expression analysis was evaluated for control (non-transduced) HEFs (FIG. 10, Bar 1), at day 7 post infection while the cells were still grown in normal culture medium (FIG. 10, Bar 2), at day 17, grown on MEF feeder cells and sorted for SSEA4 positive staining (FIG. 10, Bar 3) and at day 30 post infection (after manual picking of colonies and clonal expansion; FIG. 10, Bar 4). HEFs did not show any expression of pluripotent marker genes before infection. At 7 days post-infection the cDNA of the 5 transduced transcription factors was first detected, accompanied by slight up-regulation of Thy1 (FIG. 10R), Col5A2 (FIG. 10S), Gene A (c-RET, FIG. 10T) and Gene B (Brachyury, FIG. 10U). Earlier reports of re-programming in adult mouse tail tip fibroblasts suggested activation of alkaline phosphatase (ALPL) and a down-regulation of Thy1, with other fibroblast differentiation marker genes. In our experiment however, we detected the steepest increase of ALPL after day 17. Moreover, the fibroblast marker genes Thy1 and Col5A2 increased markedly at day 7 post infection, but then decreased again at day 17 in the SSEA4(+) population. While Col5A2 decreased below the level of control HEFs in colony forming cells at day 30, Thy1 decreased to the control level of expression in untreated HEFs.

Levels of cDNA expression of pluripotent genes in the SSEA4(+) population at day 17 were greater than in the overall cell population at day 7, show a progressive enrichment, with time, of the virus expressing cells. As there was small but distinguishable up-regulation of a number of pluripotent marker genes including hTERT (FIG. 10Q), ALPL (FIG. 10K), Cripto (FIG. 10N), Sall4 (FIG. 10M) and Dppa5 (FIG. 10L), the results show that either a small population of cells within the SSEA4(+) population were de-differentiated, or alternatively, the beginning of a global change in expression at day 30. In summary, HEF colonies expressed a complete set of endogenous pluripotent marker genes at day 30, indicating that these cells were de-differentiated.

Interestingly, c-RET (FIG. 10T, Gene A) and Brachyury (FIG. 10U, Gene B) were up-regulated early in the re-programming process in HEF iPS cells, while there is no activation of those genes in RPE cells. Only after re-infection of Nanog and c-Myc expressing cells with the missing factors, there was a slight activation of Brachyury and only a minor increase in expression in c-RET. C-RET is the receptor for GDNF and Brachyury has previously been reported to be associated with mesenchymal cell differentiation.

Skin fibroblasts and keratinocytes are reprogrammed for cell growth and expansion in tissue culture by lentiviral or SWNT introduction of just Oct-4 and hTERT, or alternatively, Oct-4 and c-Myc.

Cells from liver, kidney, lung, muscle, pancreas, bone marrow, bladder, testes and ovary are reprogrammed for cell growth and expansion in tissue culture by lentiviral or SWNT introduction of a pair of transcription factors: i.e., (a) Oct-4 and hTERT, or alternatively, (b) Oct-4 and c-myc, or alternatively, (c) Nanog and c-Myc, or alternatively, (d) Nanog and hTERT.

### Example 15

### Non-Viral Reprogramming Using Pluripotency Factor DNA or Protein and De-Methylating and Acetylating Agents

The reprogramming process using virus is typically ineffective with an approximate efficiency of 0.001%, mostly due to the lack of acetylation on the histone and trimethylation of the histones. To provide for an environment of de-methylated and acetylated histones to facilitater access of the delivered pluripotency factors, compounds that promote acetylation and de-methylation are added to the cell culture during the reprogramming process. These compounds include family members of 2-propylpentanoic acid (valproic acid, VPA) and its derivatives including, but not limited to, valproate semisodium and sodium valproate. Valproic acid is an inhibitor of histone de-acetylase and of glycogen synthase kinase 3 and thereby promotes acetylation and inhibits a molecular complex that is essential for degrading the beta catenin protein. Beta catenin, if overexpressed, inhibits differentiation and is thereby further supporting the reprogramming process. 5-Azacytidine (5-Aza) is a compound that reduces methylation by replacing cytidine and cannot be methylated.

In one non-limiting example, 2 mM VPA and 50 μM 5-Aza are added to a continuous somatic cell culture on day 1 and the cells are cultivated for 5 days. On day 6, the cells are transfected transiently with a combination of Oct-4, Sox-2, KLF4, c-Myc and Lin28 cDNA-containing mammalian expression plasmids. Alternatively the same factors are used as recombinant protein and delivered into the cell by means of SWNT (see Example 10) or polyethylene-imine particles (see Example 10). On day 7, 5-Aza and VPA are again added to the cell culture and the cells are cultivated until day 9. On day 9, 5-Aza and VPA are withdrawn and the cells are transfected again with the same pluripotency factors in form of either DNA or protein. On day 10, 5-Aza and VPA are added for the third time in the above mentioned concentrations and the cells are transferred onto a feeder layer of mitomycin C-inactivated MEFs. The cells are cultivated in hESC medium and morphological changes are observed over the next 14 days.

In a separate experiment the same factors are produced recombinantly in bacteria with a cell penetrating peptide attached to them and added to the growth medium of the cells at a concentration of 2 μM total protein concentration on day 5 of cell culture. The cells are then transferred onto MEF feeder layer and cultivated in hESC medium on day 7-10.

Colonies which display the morphological characteristics of hESC cells are stained for the surface markers SSEA-4, TRA1-60 or TRA1-80, thereby confirming a reprogramming event. The colonies are then manually removed from the culture and cultivated under standard embryonic stem cell conditions. Colonies which are expanding and still display the expression of the cell surface molecules SSEA4, TRA1-60 or TRA1-81 are clonally expanded and tested for their gene expression profile using multiplex PCR. Furthermore, cells are tested for their differentiation potential as outlined in Examples 6-98. As a test for pluripotency these cells are also tested in a teratoma transplantation experiment.

### Example 16

### Human Nanog Promoter Reporter Construct

Nanog is a transcription factor critically involved with self-renewal of undifferentiated embryonic stem cells (ESCs). It also has a role in maintaining pluripotency and works together with other transcription factors, namely Oct-4 and Sox2, in a regulatory circuitry that establishes ESC identity. Although very important with respect to ESC biology, Nanog has been shown to be dispensable for virus-mediated induced pluripotent stem cells (iPS). The factors necessary to produce iPS were Oct-4, Sox2, c-Myc, and Klf4. However, Nanog was expressed in these iPS cells. Due to these observations, it was hypothesized to use the promoter region of the Nanog gene as a promoter-reporter system. The Nanog promoter sequence that was chosen is a 500 nucleotide sequence that starts 500 nucleotides upstream (−500) relative to the transcriptional start site (+1) in the complete Nanog gene (SEQ ID NO: 8). PCR methods were used to add restriction endonuclease sites to the Nanog promoter sequence. A Xhol (CTCGAG) site was added to the forward 5′ primer and a HindIII (AAGCTT) site was added to the reverse 3′ primer. The Nanog promoter sequence was amplified using PCR and the chimeric primers explained above. The amplified PCR product and a promoter-less expression vector were digested with Xhol and HindIII. The Nanog promoter sequence was then subcloned into the promoter-less expression vector. The resulting construct was pNanog Promoter-RFP. The Nanog promoter drives expression of RFP when the promoter is activated.

(SEQ ID NO: 8)
CCAGGTTCAAGGGATTCTCCCGCCTCAGCTTCCAGAGTAGCTGGGACTAC
AGACACCCACCACCATGCGTGGCTAATTTTTGTATTTTTAGTAGAGAGGG
GGTTTCGCCATGTTGGCCAGGCTGGTTTCAAACTCCTGACTTCAGGTGAT
CCGCCTGCCACGGCCTCCCAATTTACTGGGATTACAGGGGTGGGCCACCG
CGCCCGGCCTTTTTCTTAATTTTTAAAAATATTAAAGTTTTATCCCATTC
CTGTTGAACCATATTCCTGATTTAAAAGTTGGAAACGTGGTGAACCTAGA
AGTATTTGTTGCTGGGTTTGTCTTCAGGTTCTGTTGCTCGGTTTTCTAGT
TCCCCACCTAGTCTGGGTTACTCTGCAGCTACTTTTGCATTACAATGGCC
TTGGTGAGACTGGTAGACGGGATTAACTGAGAATTCACAAGGGTGGGTCA
GTAGGGGGTGTGCCCGCCAGGAGGGGTGGGTCTAAGGTGATAGAGCCTTC

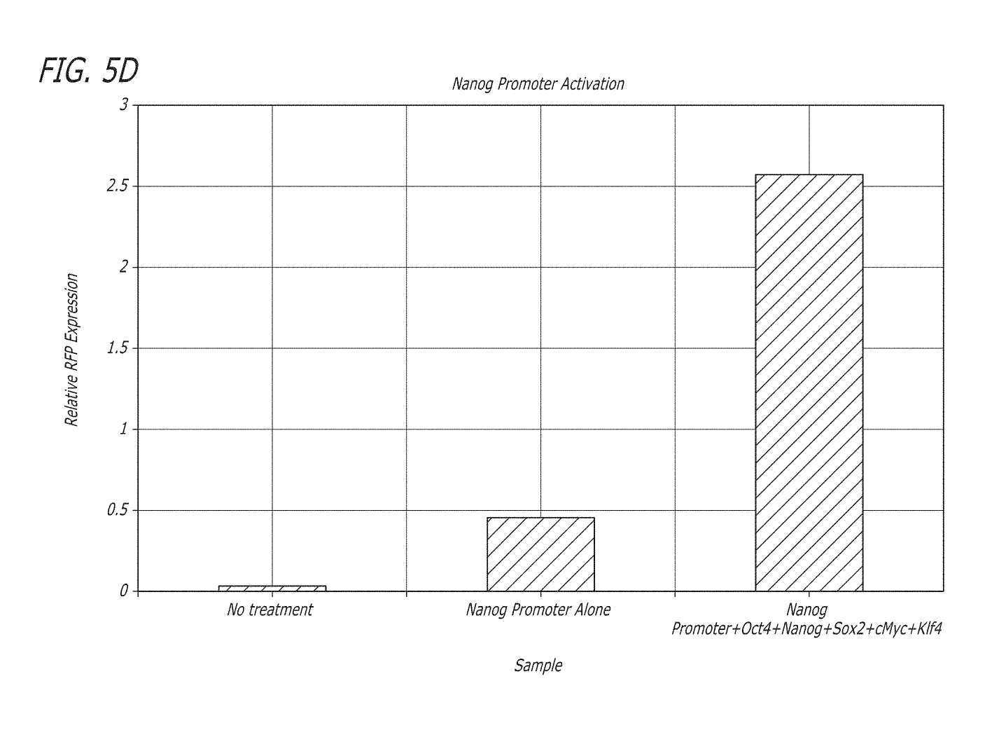

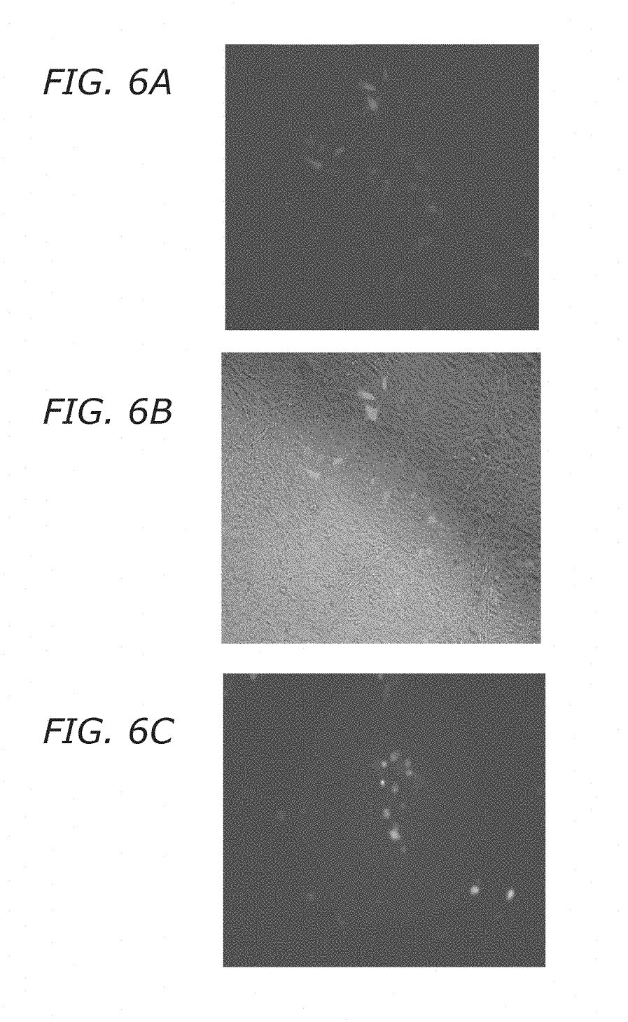

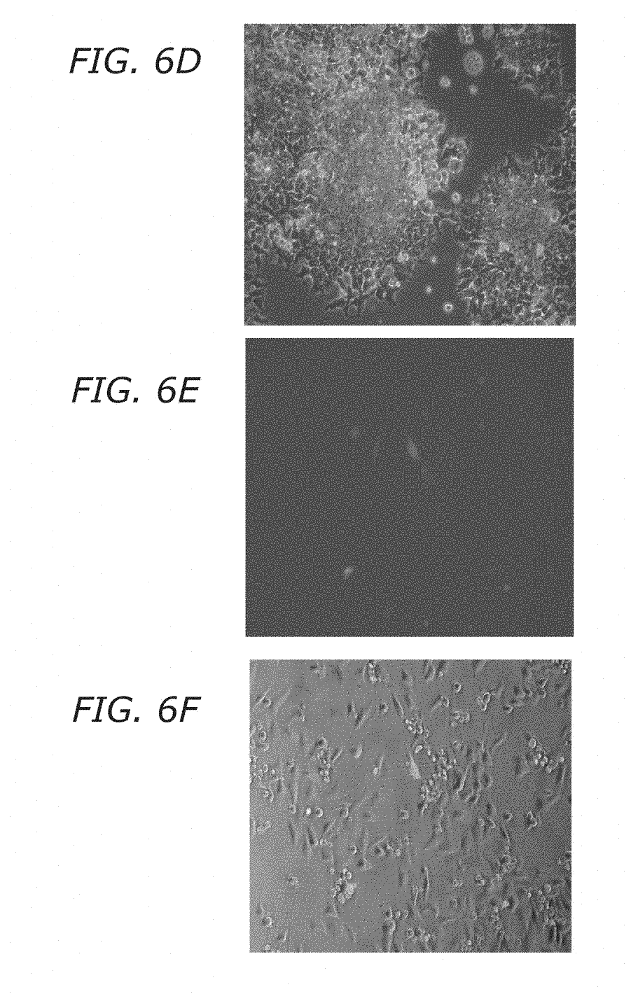

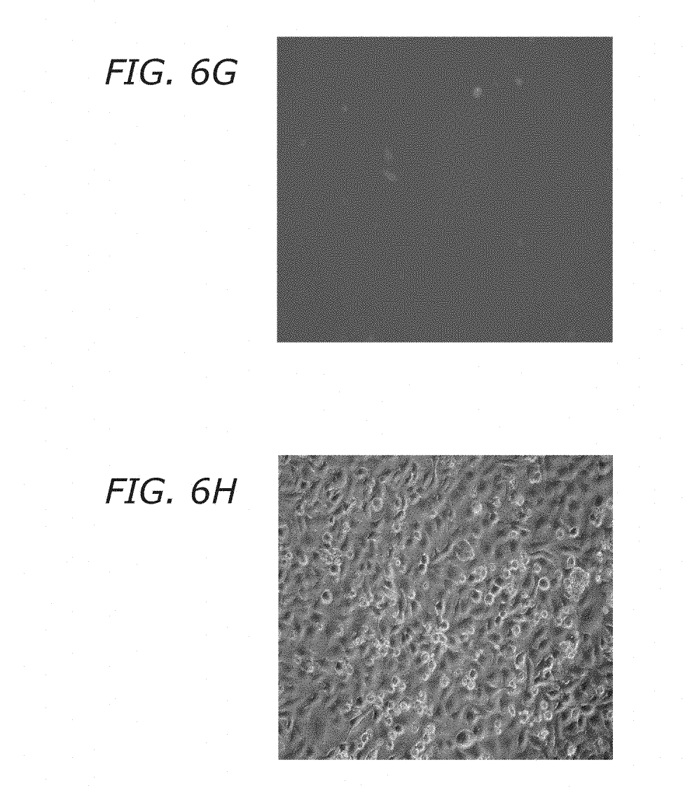

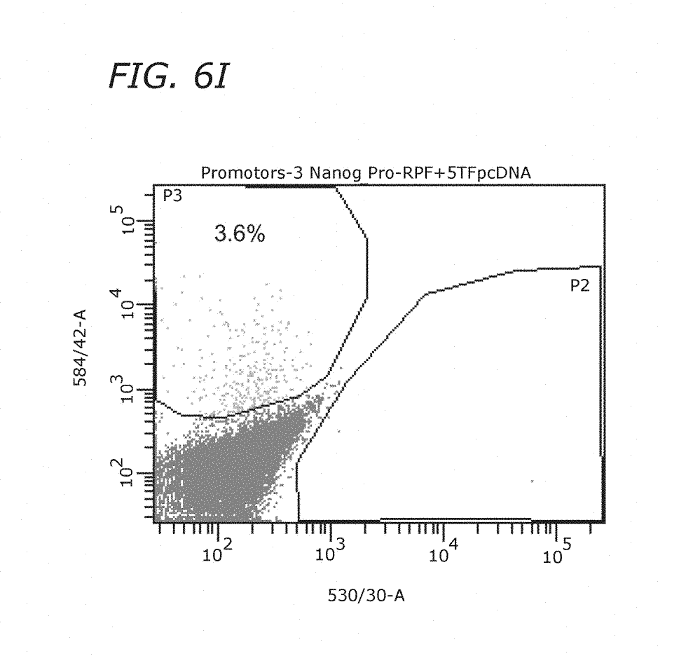

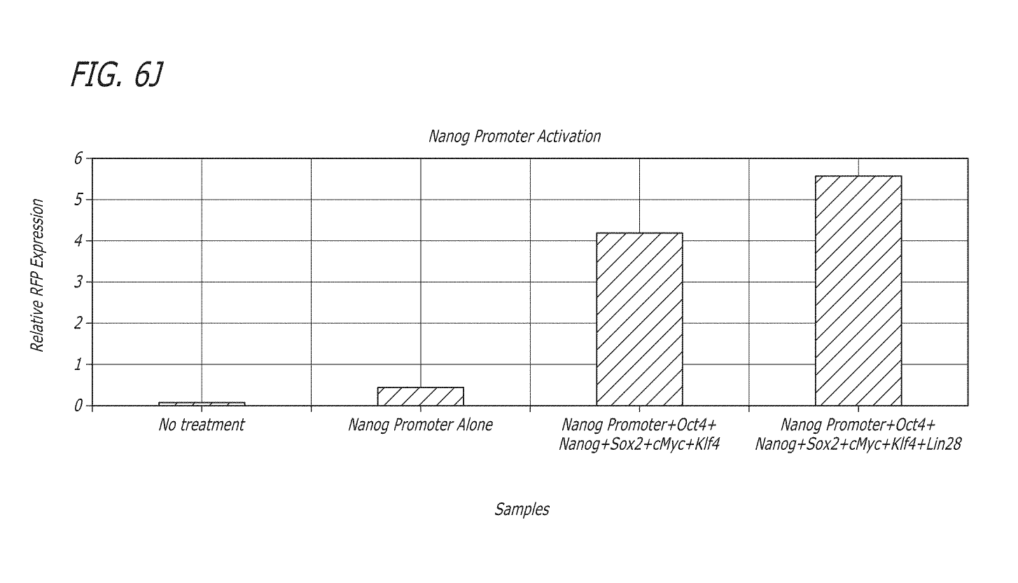

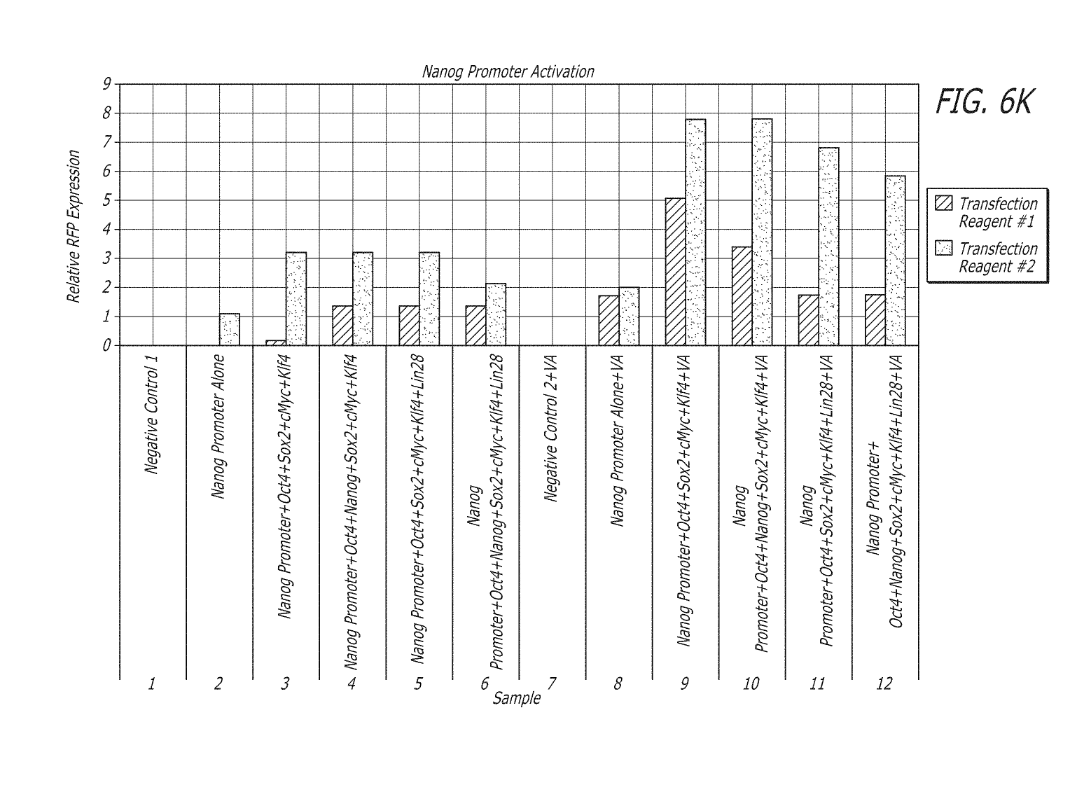

To verify that the Nanog promoter could be used as a reporter, it was transfected into two cells lines that were already pluripotent, NCCIT (teratocarcinoma cell line, ATCC) and ESCs and both RFP and Merge expression were detected (FIG. 6A).

To verify control of the Nanog promoter, HeLa cells were co-transfected with combinations of the Nanog promoter construct and reprogramming factor (RF) DNA constructs. Approximately 500 ng total DNA was transfected into HeLa cells plated on 12-well dishes at 90,000 cells per well. Four hours after transfection, the cells were washed with PBS and medium was replaced. The cells were monitored for RFP expression at 24 hr post-transfection using fluorescent microscopy and at 48 hr post-transfection using fluorescent microscopy and fluorescence activated cell sorting (FACS). FIG. 6B depicts that the Nanog promoter can be activated by co-transfecting cells with reprogramming factor DNA constructs

In a separate experiment, the Nanog promoter activation was increased over 9-fold when co-transfected with Oct-4, Nanog, Sox2, c-Myc and Klf4 compared to promoter alone. Promoter activation increased over 12-fold when Lin28 was added to the co-transfection mix (FIG. 6C).

Different combinations of reprogramming factors were then examined to determine how efficient they were at activating the Nanog promoter. Valproic acid was also tested to see the effects it may have on the Nanog promoter activation. HeLa cells were plated in 12-well dishes at 90,000 cells per well. Approximately 24 hr later, after allowing cells to attach and spread, cells were subjected to chemical transfection. Wells were transfected with different combinations of RFs (Oct-4, Nanog, Sox2, c-Myc, Klf4, Lin28) along with a construct with RFP driven by the Nanog promoter. Separate wells were left untransfected or with a construct containing the CMV promoter driving RFP (RFP positive expression control). All conditions described were performed with and without valproic acid (VPA, final concentration at 2 uM), where VPA was added 24 hours after transfection. At 48 hr post transfection; cells were harvested and subjected to FACS to analyze cells for RFP expression. In one experiment; Nanog promoter activation was increased by 39-fold when VPA was introduced to the culture 24 hr post transfection (FIG. 6D, sample #9 vs. #3). Also, there was an increase in activation when VPA was added to the culture with other combinations of RF DNA constructs; 2.5 fold increase (FIG. 6D, sample #10 vs. #4), and 1.3 fold increase (FIG. 6D, sample #11 vs. #5 and sample #12 vs. #6).

Using a standard lipofectamine transfection method, the same trend was observed with the addition of VPA. There was a 2.5 fold increase with addition of VPA (FIG. 6D, sample #9 vs. #3 and sample #10 vs. #4). There was also an increase in promoter activation with VPA treatment in two other conditions (FIG. 6D, sample #11 vs. #5 and sample #12 vs. #6).

In summary, transient transfection of the Nanog promoter leads to Nanog activation in pluripotent cell lines, co-transfection of multiple pluripotency factors (transcription factors) and the Nanog promoter leads to activation of the Nanog promoter in non-pluripotent cell lines and valproic acid has an enhancing effect on transient Nanog expression. Transient delivery of pluripotency factors, with or without valproic acid has the effect of non-virally reprogramming somatic or germ-line cells into multipotent or pluripotent cells.

### Example 17

### CPP-Mediated Expression of Oct-4

Newborn human foreskin fibroblast (BJ) cells were treated with pyrene butyrate (PB) in PBS or basal medium (FIGS. 11A and 11B, respectively) or no pretreatment (FIG. 11C), followed by staining with Oct4-TAT labeled with Alexa-Fluor 488 (Oct4-TAT-488) and then imaged at 10× magnification. The cells were imaged using brightfield (BF), blue fluorescence (DAPI, which detects PB), green fluorescence (GFP, for imaging Alexa-Fluor-488 conjugated to Oct4-TAT), and red fluorescence (TR, control to ensure green and blue fluorescence are not auto-fluorescent).

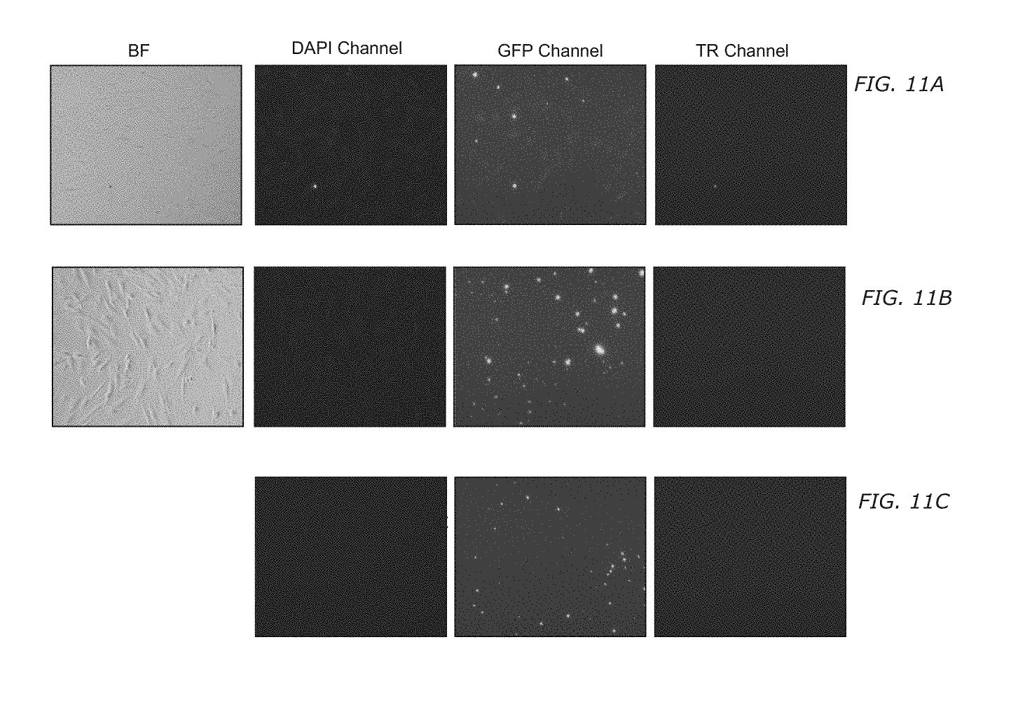

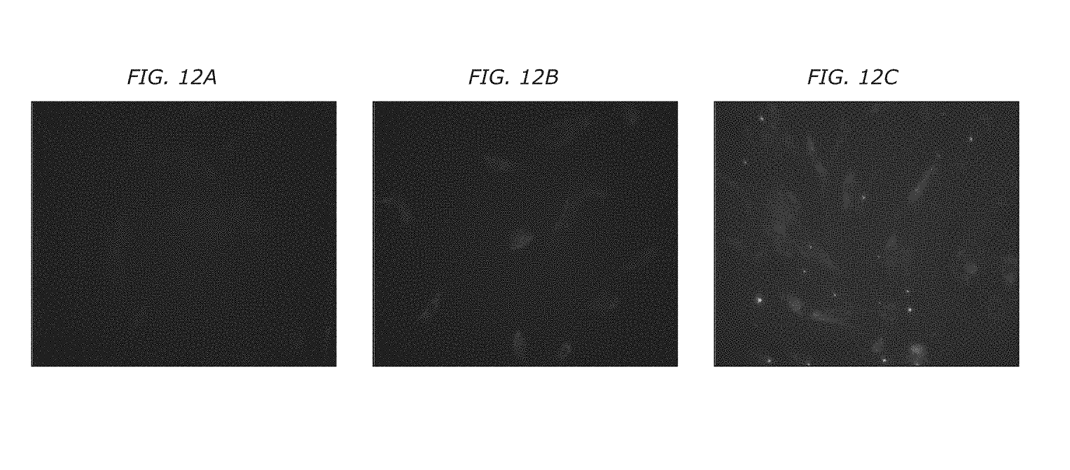

The BJ cells display a ubiquitous Alexa-Fluor 488 signal after PB treatment in PBS in FIG. 11A and less so with no pretreatment in FIG. 11C or PB treatment in basal medium in FIG. 11B. FIG. 12A-C shows an identical experiment, except that the cells were fixed following treatment with Oct4-TAT-488. The same pattern was observed on fixed cells imaged at 20× magnification.

### Example 18

### SSEA-4 Positive Colonies as a Result of 5TF-TAT Treatment

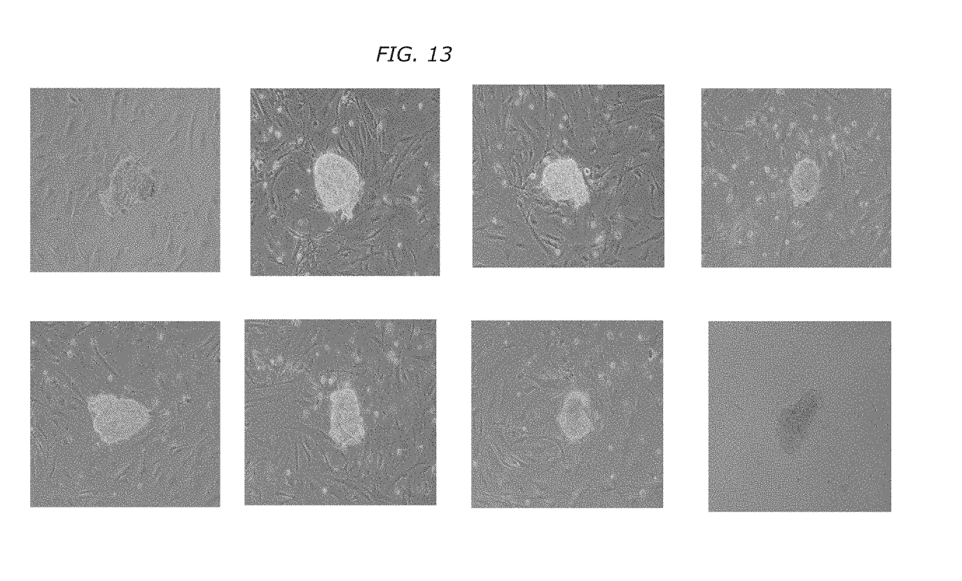

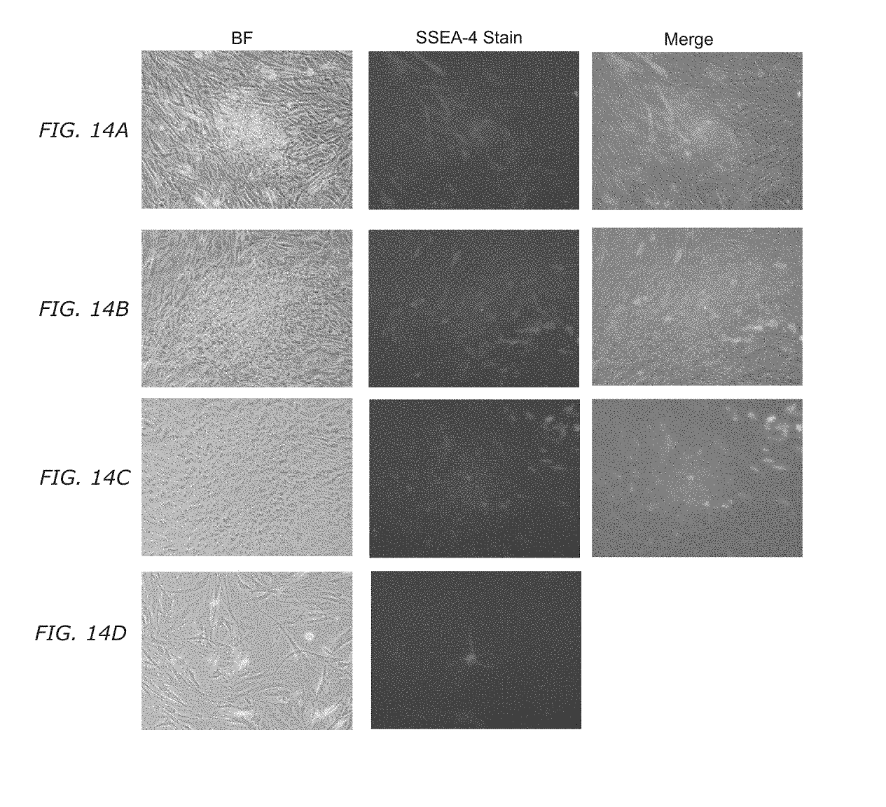

BJ cells were treated with PB+5TF-TAT mix for 2 hours, every other day for 10 total protein transductions. Valproic acid (VPA) was used to pretreat the cells on Day −1, removed, and added again at Day 9 (at a concentration of 0.125 mM). At Day 28, colonies were present and stained for SSEA-4, picked, transferred to a new dish, and allowed to attach; FIG. 13 shows colony morphology approximately 15 hours after picking and transfer. FIG. 14A-D shows colonies that stained positively for stem cell marker SSEA-4 (labeled as “488” positive). Negative control images (FIG. 14A) were taken from a well of BJ cells not treated with the 5TF-TAT mixture. FIGS. 14A-C are representative colonies generated from the 5TF-TAT transduction experiment. Images are at 10× magnification and captured at Day 28.

Thus, the five pluripotency factors of Oct-4, c-Myc, Sox-2, Klf-4 and Nanog, each associated with a molecule that facilitates entry of the pluripotency factors into the cell, can induce transformation of newborn human foreskin fibroblast (BJ) cells from a non-pluripotent phenotype to a pluripotent phenotype.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method for producing reprogrammed cells comprising the steps of:
isolating a cell from a subject;
introducing a mixture of pluripotency factors into the cell, wherein the pluripotency factors consist of Oct-4, c-Myc, Sox-2, Klf-4, and Nanog, and wherein each of the pluripotency factors are individually associated with a cell penetrating peptide that facilitates entry of the pluripotency factors into the cell;
thus reprogramming the cells to express at least one embryonic stem cell marker selected from the group consisting of Oct-4, Nanog, SSEA-3, SSEA-4, TRA1-60, Stellar, alkaline phosphatase, VASA, cRET, and Rex-1; and
determining that greater than 5% of the reprogrammed cells express the at least one embryonic stem cell marker.

2. The method of claim 1, wherein the cell is selected from the group consisting of somatic cells, germ cells, and post-natal stem cells.

3. The method of claim 1, wherein the reprogrammed cell can differentiate into multiple cell lineages.

4. The method of claim 1 further comprising the step of incubating the cell under conditions suitable for growth and progeny cell formation to form a continuous cell line.

5. The method of claim 1, further comprising addition of at least one of a demethylation agent and/or at least one of an acetylation agent in the introducing step.

6. The method of claim 5, wherein the acetylation agent comprises valproic acid or a derivative thereof.

7. The method of claim 1, wherein the subject is suffering from age-related macular degeneration; Type-1 insulin-dependent diabetes mellitus (IDDM); Type-2 diabetes; bone marrow reconstitution; non-union bone fractures; cosmetic clinical indications; infertility; Parkinson's disease; multiple sclerosis; amyotrophic lateral sclerosis (ALS); Alzheimer's disease; cystic fibrosis; fibromyalgia; cosmetic and reconstructive surgery for skin; cartilage and bone; myocardial infarct; stroke, spinal cord injury; traumatic injury; and restoring, regenerating and rejuvenating damaged and aged tissues.

8. The method of claim 1, wherein the method further comprises the steps of:
passaging the reprogrammed cells to obtain a therapeutically effective amount of reprogrammed cells; and
administering the therapeutically effective amount of reprogrammed cells to the subject.

9. A method of treatment comprising:
isolating a cell from a subject;
introducing a mixture of pluripotency factors into the cell,
wherein the pluripotency factors consist of Oct-4, c-Myc, Sox-2, Klf-4, and Nanog, wherein each of the pluripotency factors are individually associated with a cell penetrating peptide that facilitates entry of the pluripotency factors into the cell;
thus reprogramming the cells to express at least one embryonic stem cell marker selected from the group consisting of Oct-4, Nanog, SSEA-3, SSEA-4, TRA1-60, Stellar, alkaline phosphatase, VASA, cRET, and Rex-1;
expanding the reprogrammed cells;
culturing the reprogrammed cells with a differentiation media until the reprogrammed cells are differentiated; and
administering a therapeutically effective amount of differentiated cells to the subject.

10. The method of claim 9, wherein the cell is selected from the group consisting of somatic cells, germ cells, and post-natal stem cells.

11. The method of claim 9, wherein the reprogrammed cells are differentiated into mesodermal tissues.

12. The method of claim 11, wherein the mesodermal tissues are neurologic tissues, cardiac tissues, connective tissue, epithelial tissues, osteogenic tissue, chondrogenic tissue, adipogenic tissue, muscle tissue, or hematopoietic tissue.

13. The method of claim 12, wherein the connective tissue is pancreatic islet cells, lung parenchymal cells, or liver hepatocytes.

14. The method of claim 12, wherein the hematopoietic tissue is bone marrow.

15. The method of claim 12, wherein the osteogenic tissue are osteocytes and osteoblasts.

16. The method of claim 12, wherein the muscle tissue is cardiomyocytes.

17. The method of claim 12, wherein the epithelial tissue is renal epithelial cells, retinal pigment epithelial cells, and proximal tubule cells.

18. A method of treatment comprising:
isolating a cell from a subject;
introducing a mixture of pluripotency factors into the cell,
wherein the pluripotency factors consist of Oct-4, c-Myc, Sox-2, Klf-4, and Nanog, wherein each of the pluripotency factors are individually associated with a cell penetrating peptide that facilitates entry of the pluripotency factors into the cell;
reprogramming the cells to express at least one embryonic stem cell marker selected from the group consisting of Oct-4, Nanog, SSEA-3, SSEA-4, TRA1-60, Stellar, alkaline phosphatase, VASA, cRET, and Rex-1;
introducing a pharmaceutically acceptable carrier to a therapeutically effective amount of reprogrammed cells; and
administering a therapeutically effective amount of reprogrammed cells to a second subject.

19. The method of claim 18, wherein the pharmaceutical acceptable carrier is a biodelivery gel, a biodegradable semi-solid matrix, a diluent, a filler, a sterile aqueous solution, or a solvent.

20. The method of claim 18, wherein the therapeutically effective amount of reprogrammed cells are administered topically, intravenously, intraperitoneally, intramuscularly, subcutaneously, intradermally, intransasally, intrabronchially, transdermally, intrathecally, rectally, or gasatrointestinally.
